(19) United States
(12) Patent Application Publication
(10) Pub. No.: US 2024/0398863 A1
(43) Pub. Date: Dec. 5, 2024

(54) CHIMERIC ANTIGEN RECEPTOR AND CELL INCLUDING CHIMERIC ANTIGEN RECEPTOR

(71) Applicant: ARCE THERAPEUTICS, INC., Taipei City (TW)

(72) Inventors: Chien-Tsun KUAN, Cary, NC (US); Kao-Jean HUANG, Taipei City (TW); Shun-Jen YANG, Taipei City (TW); Tsung-Han WU, Taipei City (TW); Hui-Chun CHEN, Taipei City (TW); Hom-Ming YEH, Taipei City (TW)

(21) Appl. No.: 18/368,516

(22) Filed: Sep. 14, 2023

Related U.S. Application Data
(60) Provisional application No. 63/406,700, filed on Sep. 14, 2022.

(51) Int. Cl. A61K 35/17; A61K 39/00; A61P 35/02; C07K 16/28

(52) U.S. Cl. CPC A61K 35/17; A61K 39/4611; A61K 39/4631; A61K 39/464402; A61P 35/02; C07K 16/2851; A61K 2239/13; A61K 2239/17; A61K 2239/21; A61K 2239/22; A61K 2239/48

(57) ABSTRACT

The present disclosure provides chimeric antigen receptor (CAR) for Human C-type lectin-like molecule-1 (CLL-1) comprising a polypeptide comprising: an extracellular antigen binding domain comprising an anti-CLL-1 single heavy chain variable domain (VH) and anti-CLL-1 single light chain variable domain (VL); a transmembrane domain; and an intracellular signaling domain. Immune effector cells comprising the CAR and pharmaceutical compositions based on the immune effector cells, as well as their therapeutic use in treating condition associated with CLL-1, are also disclosed.

### CROSS-REFERENCE OF RELATED APPLICATIONS

This application claims priority under 35 U.S.C. 119 of U.S. Provisional Application No. 63/406,700, filed on Sep. 14, 2022, which is incorporated by reference herein.

### BACKGROUND

Effector cell activities can involve a ligand binding to a membrane-bound receptor that comprises an extracellular antigen binding domain and an intracellular signaling domain. The formation of a complex between the antigen binding domain and its corresponding target can result in a conformational and/or chemical modification to the receptor itself, which in turn can yield an array of signals transduced within the cell. Attempts have been made to harness this interaction for the development of immune cell therapies.

### SUMMARY

The various aspects of the disclosure provide systems, compositions, and methods for inducing activity of immune cells.

In an aspect, the present disclosure provides a chimeric antigen receptor (CAR) for Human C-type lectin-like molecule-1 (CLL-1) comprising a polypeptide comprising: an extracellular antigen binding domain comprising a single heavy chain variable domain (VH) and a single light chain variable domain (VL); a transmembrane domain; and an intracellular signaling domain, wherein the single heavy chain variable domain comprises a CDR1, a CDR2 and a CDR3 as set forth in a first amino acid sequence selected from the group consisting of SEQ ID NO: 10, SEQ ID NO: 12, SEQ ID NO: 14, SEQ ID NO: 16, and SEQ ID NO: 18, and wherein the single light chain variable domain comprises a CDR1, a CDR2, and a CDR3 as set forth in a second amino acid sequence selected from the group consisting of SEQ ID NO: 11, SEQ ID NO: 13, SEQ ID NO: 15, SEQ ID NO: 17, and SEQ ID NO: 19.

In some embodiments, the first amino acid sequence is of SEQ ID NO: 14. In some embodiments, the second amino acid sequence is of SEQ ID NO: 15.

In some embodiments, the single heavy chain variable domain is located at a N-terminus side of the single light chain variable domain. In some embodiments, the single heavy chain variable domain is located at a C-terminus side of the single light chain variable domain.

In some embodiments, the single heavy chain variable domain and single light chain variable domain are directly fused to each other via a peptide bond.

In some embodiments, the single heavy chain variable domain and the single light chain variable domain are linked to each other via a peptide linker. In some embodiments, the peptide linker comprises no more than 50 amino acid residues.

In some embodiments, the transmembrane domain is derived from CD8 or CD28.

In some embodiments, the intracellular signaling domain comprises a primary intracellular signaling domain of an immune effector cell. In some embodiments, the primary intracellular signaling domain is derived from CD3.

In some embodiments, the intracellular signaling domain comprises a co-stimulatory signaling domain. In some embodiments, the co-stimulatory signaling domain is derived from a molecule selected from the group consisting of CD28, 4-1BB, OX40, ICOS and combinations thereof. In some embodiments, the co-stimulatory signaling domain is derived from CD28, 4-1BB, or a combination thereof. In some embodiments, the co-stimulatory signaling domain comprises a cytoplasmic domain of CD28.

In some embodiments, the CAR further comprises a hinge domain. In some embodiments, the hinge domain is located between a C-terminus of the extracellular antigen binding domain and a N-terminus of the transmembrane domain. In some embodiments, the hinge domain is derived from molecule selected from the group consisting of CD28, 4-1BB, OX40, ICOS and combinations thereof. In some embodiments, the hinge domain is derived from CD28.

In some embodiments, the CAR further comprises a signal peptide located at a N-terminus of the polypeptide. In some embodiments, the signal peptide is derived from CD28.

In another aspect, the present disclosure provides a chimeric antigen receptor (CAR) for Human C-type lectin-like molecule-1 (CLL-1) comprising a polypeptide comprising: an extracellular antigen binding domain comprising a single heavy chain variable domain (VH) and a single light chain variable domain (VL); a transmembrane domain; and an intracellular signaling domain, wherein the single heavy chain variable domain comprises a CDR1, a CDR2 and a CDR3 as set forth in an amino acid sequence of SEQ ID NO: 14, and wherein the single light chain variable domain comprises a CDR1, a CDR2, and a CDR3 as set forth in an amino acid sequence of SEQ ID NO: 15.

In some embodiments, the single heavy chain variable domain is located at a N-terminus side of the single light chain variable domain. In some embodiments, the single heavy chain variable domain is located at a C-terminus side of the single light chain variable domain.

In some embodiments, the single heavy chain variable domain and single light chain variable domain are directly fused to each other via a peptide bond.

In some embodiments, the single heavy chain variable domain and the single light chain variable domain are linked to each other via a peptide linker. In some embodiments, the peptide linker comprises no more than 50 amino acid residues.

In some embodiments, the transmembrane domain is derived from CD8 or CD28.

In some embodiments, the intracellular signaling domain comprises a primary intracellular signaling domain of an immune effector cell. In some embodiments, the primary intracellular signaling domain is derived from CD3ζ.

In some embodiments, the intracellular signaling domain comprises a co-stimulatory signaling domain. In some embodiments, the co-stimulatory signaling domain is derived from a molecule selected from the group consisting of CD28, 4-1BB, OX40, ICOS and combinations thereof. In some embodiments, the co-stimulatory signaling domain is derived from CD28, 4-1BB, or a combination thereof. In some embodiments, the co-stimulatory signaling domain comprises a cytoplasmic domain of CD28.

In some embodiments, the CAR further comprises a hinge domain. In some embodiments, the hinge domain is located between a C-terminus of the extracellular antigen binding domain and a N-terminus of the transmembrane domain. In some embodiments, the hinge domain is derived from molecule selected from the group consisting of CD28, 4-1BB, OX40, ICOS and combinations thereof. In some embodiments, the hinge domain is derived from CD28.

In some embodiments, the CAR further comprises a signal peptide located at a N-terminus of the polypeptide. In some embodiments, the signal peptide is derived from CD28.

In another aspect, the present disclosure provides a chimeric antigen receptor (CAR) for Human C-type lectin-like molecule-1 (CLL-1) comprising a polypeptide comprising: an extracellular antigen binding domain comprising an anti-CLL-1 single heavy chain variable domain (VH) and anti-CLL-1 single light chain variable domain (VL); a transmembrane domain derived from CD8, CD28, 4-1BB, or a combination thereof; and an intracellular signaling domain is derived from CD8, CD28, 4-1BB, OX40, ICOS, or a combination thereof.

In some embodiments, the anti-CLL-1 single heavy chain variable domain comprises a CDR1, a CDR2 and a CDR3 as set forth in a first amino acid sequence selected from the group consisting of SEQ ID NO: 10, SEQ ID NO: 12, SEQ ID NO: 14, SEQ ID NO: 16, and SEQ ID NO: 18.

In some embodiments, the anti-CLL-1 single light chain variable domain comprises a CDR1, a CDR2, and a CDR3 as set forth in a second amino acid sequence selected from the group consisting of SEQ ID NO: 11, SEQ ID NO: 13, SEQ ID NO: 15, SEQ ID NO: 17, and SEQ ID NO: 19.

In some embodiments, the anti-CLL-1 single heavy chain variable domain comprises a CDR1, a CDR2 and a CDR3 as set forth in the VH domain comprising an amino acid sequence of SEQ ID NO: 14.

In some embodiments, the anti-CLL-1 single light chain variable domain comprises a CDR1, a CDR2, and a CDR3 as set forth in the VL domain comprising an amino acid sequence of SEQ ID NO: 15.

In some embodiments, the single heavy chain variable domain is located at a N-terminus side of the single light chain variable domain. In some embodiments, the single heavy chain variable domain is located at a C-terminus side of the single light chain variable domain.

In some embodiments, the single heavy chain variable domain and single light chain variable domain are directly fused to each other via a peptide bond.

In some embodiments, the single heavy chain variable domain and the single light chain variable domain are linked to each other via a peptide linker. In some embodiments, the peptide linker comprises no more than 50 amino acid residues.

In some embodiments, the transmembrane domain is derived from CD8 or CD28.

In some embodiments, the intracellular signaling domain comprises a primary intracellular signaling domain of an immune effector cell. In some embodiments, the primary intracellular signaling domain is derived from CD3ζ.

In some embodiments, the intracellular signaling domain comprises a co-stimulatory signaling domain. In some embodiments, the co-stimulatory signaling domain is derived from a molecule selected from the group consisting of CD28, 4-1BB, OX40, ICOS and combinations thereof. In some embodiments, the co-stimulatory signaling domain is derived from CD28, 4-1BB, or a combination thereof. In some embodiments, the co-stimulatory signaling domain comprises a cytoplasmic domain of CD28.

In some embodiments, the CAR further comprises a hinge domain. In some embodiments, the hinge domain is located between a C-terminus of the extracellular antigen binding domain and a N-terminus of the transmembrane domain. In some embodiments, the hinge domain is derived from molecule selected from the group consisting of CD28, 4-1BB, OX40, ICOS and combinations thereof. In some embodiments, the hinge domain is derived from CD28.

In some embodiments, the CAR further comprises a signal peptide located at a N-terminus of the polypeptide. In some embodiments, the signal peptide is derived from CD28.

In another aspect, the present disclosure provides a chimeric antigen receptor (CAR) for Human C-type lectin-like molecule-1 (CLL-1) comprising: a polypeptide comprising an amino acid sequence selected from the group consisting of SEQ ID NOs: 20 to 26. In some embodiments, the polypeptide comprises amino acid sequence is selected from the group consisting of SEQ ID NOs: 25 to 26.

In another aspect, the present disclosure provides a chimeric antigen receptor (CAR) for Human C-type lectin-like molecule-1 (CLL-1) comprising: a polypeptide derived from nucleic acid sequence selected from the group consisting of SEQ ID NOs: 27 to 33. In some embodiments, the polypeptide comprises nucleic acid sequence is selected from the group consisting of SEQ ID NOs: 32 to 33.

In another aspect, the present disclosure provides a chimeric antigen receptor (CAR) for Human C-type lectin-like molecule-1 (CLL-1) comprising a polypeptide comprising: an extracellular antigen binding domain comprising a single heavy chain variable domain (VH) and a single light chain variable domain (VL); a transmembrane domain; and an intracellular signaling domain, wherein the single heavy chain variable domain comprises a CDR1, a CDR2 and a CDR3 as set forth in a first amino acid sequence selected from the group consisting of SEQ ID NO: 10, SEQ ID NO: 12, SEQ ID NO: 14, SEQ ID NO: 16, and SEQ ID NO: 18, or wherein the single light chain variable domain comprises a CDR1, a CDR2, and a CDR3 as set forth in a second amino acid sequence selected from the group consisting of SEQ ID NO: 11, SEQ ID NO: 13, SEQ ID NO: 15, SEQ ID NO: 17, and SEQ ID NO: 19.

In some embodiments, the first amino acid sequence is of SEQ ID NO: 14. In some embodiments, the second amino acid sequence is of SEQ ID NO: 15.

In some embodiments, the single heavy chain variable domain is located at a N-terminus side of the single light chain variable domain. In some embodiments, the single heavy chain variable domain is located at a C-terminus side of the single light chain variable domain.

In some embodiments, the single heavy chain variable domain and single light chain variable domain are directly fused to each other via a peptide bond.

In some embodiments, the single heavy chain variable domain and the single light chain variable domain are linked to each other via a peptide linker. In some embodiments, the peptide linker comprises no more than 50 amino acid residues.

In some embodiments, the transmembrane domain is derived from CD8 or CD28.

In some embodiments, the intracellular signaling domain comprises a primary intracellular signaling domain of an immune effector cell. In some embodiments, the primary intracellular signaling domain is derived from CD3ζ.

In some embodiments, the intracellular signaling domain comprises a co-stimulatory signaling domain. In some embodiments, the co-stimulatory signaling domain is derived from a molecule selected from the group consisting of CD28, 4-1BB, OX40, ICOS and combinations thereof. In some embodiments, the co-stimulatory signaling domain is derived from CD28, 4-1BB, or a combination thereof. In some embodiments, the co-stimulatory signaling domain comprises a cytoplasmic domain of CD28.

In some embodiments, the CAR further comprises a hinge domain. In some embodiments, the hinge domain is located between a C-terminus of the extracellular antigen binding domain and a N-terminus of the transmembrane domain. In some embodiments, the hinge domain is derived from molecule selected from the group consisting of CD28, 4-1BB, OX40, ICOS and combinations thereof. In some embodiments, the hinge domain is derived from CD28.

In some embodiments, the CAR further comprises a signal peptide located at a N-terminus of the polypeptide. In some embodiments, the signal peptide is derived from CD28.

In another aspect, the present disclosure provides an immune effector cell comprising the CAR disclosed herein. In some embodiments, the immune effector cell is a T cell.

In another aspect, the present disclosure provides a pharmaceutical composition comprising the immune effector cell disclosed herein, and a pharmaceutically acceptable carrier.

In another aspect, the present disclosure provides a method of treating a cancer that expresses CLL-1 in an individual, comprising administering to the individual an effective amount of the immune effector cell or the pharmaceutical composition disclosed herein.

In some embodiments, the cancer is multiple myeloma. In some embodiments, the cancer is refractory or relapsed multiple myeloma.

In some embodiments, the cancer is myeloid leukemia. In some embodiments, the cancer is refractory or relapsed myeloid leukemia.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features of the disclosure are set forth with particularity in the appended claims. A better understanding of the features and advantages of the present invention will be obtained by reference to the following detailed description that sets forth illustrative embodiments, in which the principles of the disclosure are utilized, and the accompanying drawings of which:

FIG. 1 illustrates screening of the anti-CLL-1 antibodies disclosed herein by flow cytometry: (A) Representative flow cytometry dot plots. (B) Overlapping histograms. Each scFv-Fc antibody was stained at 0.2 nM with paired K562-CLL-1 and K562 cells, followed by staining with fluorochrome-conjugated 2nd antibody for flow cytometry analysis;

FIG. 2 illustrates binding of disclosed anti-CLL-1 antibodies to AML cell lines;

FIG. 3A-3B illustrates the Kd values of top 3 flow cytometry screened anti-CLL-1 scFv-Fc clones;

FIG. 4 illustrates design and construction of CLL-1 CAR genes with the advanced anti-CLL antibody hits;

FIG. 5 illustrates CLL-1 (61H08) CAR genes with scFv VH-VL swapping and co-stimulatory domain exchange;

FIG. 6A-C illustrate productivity assessment of CLL-1 CAR-T cells derived from donor 25: (A) T cell viability. (B) CAR %. The transduction efficiency of CAR-T cells was assayed by EGFP expression (EGFP %). (C) CAR-T cell count;

FIG. 7A-C illustrate productivity assessment of CLL-1 CAR-T cells derived from donor 26: (A) T cell viability. (B) CAR %. The transduction efficiency of CAR-T cells was assayed by EGFP expression (EGFP %). (C) CAR-T cell count.

FIG. 8A-F illustrate phenotypic profiles of CLL-1 CAR-T cells from donor 25 and 26: (A, D) The CD4 and CD8 populations of CAR-T cell clones were analyzed by flow cytometry on day 6. (B, E) CAR-T cell phenotype. T cell subsets were defined as Tₙₐᵢᵥₑ (CCR7+CD45RA+CD95−), T_{scm} (CCR7+CD45RA+CD95+), T_{cm} (CCR7+CD45RA−), Tₑₘ (CCR7−CD45RA−) and T_{eff} (CCR7−CD45RA+CD95+). (C, F) Inhibitory marker expression (PD-1, TIM-3 and LAG-3);

FIG. 9A-B illustrate CAR expression profiles of CLL-1 CAR-T cells from donor 25 and 26: (A) CAR expression profiles of donor 25-derived CLL-1 CAR-T cells. (B) CAR expression profiles of donor 26-derived CLL-1 CAR-T cells. The transduction efficiency of CAR-T cells was assayed with EGFP expression (EGFP %) and CLL-1-ECD antigen-labelling method, and the CAR expression levels were revealed by the MFI (Median Fluorescent Intensity);

FIG. 10A-B illustrate the cytotoxicity of CLL-1 CAR-T cell clones against CLL-1+U937 cells: (A) Donor 25-derived CLL-1 CAR-T cell-mediated cytotoxicity. (B) Donor 26-derived CLL-1 CAR-T cell-mediated cytotoxicity. Individual CAR-T cell clones were incubated with luciferase-engineered AML U937 cells at the E/T ratios of 0.25, 0.5, 1, 2, 4 and 8 for 6 hrs. The luciferase activity of the remaining cell lysates after adding the luciferin solution was measured using Cytation™ 5 Cell Imaging Multi-Mode Reader. The specific lysis was calculated from the data according to the formula: % specific lysis=100×(experimental release−spontaneous release)/(maximum release−spontaneous release);

FIG. 11A-B illustrate the in vitro cytokine release profiles of CLL-1 CAR-T Cell Clones encountered with CLL-1+U937 cells: (A) Donor 25-derived CLL-1 CAR-T cells released cytokines. (B) Donor 26-derived CLL-1 CAR-T cells released cytokines. The CLL-1 CAR-T cell clones were co-cultured with U937 cells at E/T=4 for 6 hrs in the cytotoxicity assay and the released cytokines in culture supernatant were measured using LEGENDplex™ Human CD8/NK Panel (13-plex);

FIG. 12A-C illustrate the BLI Data of CLL-1 CAR-T Cell Clones in U937 Xenograft Model: (A) Tumor burden spider plots of xenograft mice receiving different CAR-T cell clones derived from Donor 25. (B) Tumor burden spider plots of xenograft mice receiving different CAR-T cell clones derived from Donor 26. (C) The BLI images represented for U937 xenograft mice receiving different CAR-T cell clones;

FIG. 13A-D illustrate the Kaplan-Meier survival curves of xenograft mice receiving CAR-T cells and the in vivo CLL-1 CAR-T cell persistence: (A, B) The Kaplan-Meier survival curves (C, D) Kinetically monitoring of CLL-1 CAR-T cells in the peripheral blood of xenograft mice;

FIG. 14A-B illustrate in vivo cytokine release profiles of xenograft mice receiving CLL-1 CAR-T cell clones: (A) Kinetic cytokine profiles in the plasma samples of mice receiving Donor 25-derived CAR-T cells. (B) Kinetic cytokine profiles in the plasma samples of mice receiving Donor 26-derived CAR-T cells. The levels of various human cytokines in the collected mice plasma samples were measured using LEGENDplex™ Human CD8/NK Panel (13-plex);

FIG. 15A-B illustrate the hematotoxicity assessment of disclosed CLL-1 CAR-T cell candidates: The hematotoxicity assessment was conducted by colony formation inhibition assay with bone marrow- or peripheral blood-derived CD34+ stem cells inoculated with the indicated CAR-T cells at E/T ratio of 1 (A) and 4 (B). To normalize, the average colony number from CD34+ group was set at 100% and the values from the other groups were adjusted using the following calculation: (average colony number of treated samples/average colony number of CD34+ samples)×100%;

FIG. 16 illustrates characteristics of processed CLL-1 CAR-T cell candidate. CLL-1 CAR-T cell candidate was produced with T cells from Donor A011 and A012. CAR percentage was defined using EGFP expression. The CD4 and CD8 populations, T cell subsets, and inhibitory marker expression of ARD103 were analyzed by flow cytometry. T cell subsets were defined as Tₙₐᵢᵥₑ (CCR7+CD45RA+CD95−), T_{scm} (CCR7+CD45RA+CD95+), T_{cm} (CCR7+CD45RA−), Tₑₘ (CCR7−CD45RA−) and T_{eff} (CCR7−CD45RA+CD95+). Inhibitory markers are PD-1, TIM-3 and LAG-3;

FIG. 17A-C illustrate CLL-1 CAR-T cell candidate-mediated cytotoxicity is correlated with CLL-1 antigen expression levels: (A) Expression levels of CLL-1 antigens on AML cell lines (U937, THP-1, HL60, MOLM-13, and MOLM14) and CML cell line (K562) were detected by flow cytometry using anti-human CLL-1-APC antibody (clone: REA431). (C) Cytolytic activity of ARD103 against AML cell lines or CML cell line at different effector to target (E: T) ratios was measured using luciferase-based reporter assay. CAR-T cells were incubated with luciferase-engineered AML cell lines at the E/T ratios of 1, 2, 4 and 8 for 4 hrs˜6 hrs. The luciferase activity of the remaining cell lysates after adding the luciferin solution was measured using Cytation™ 5 Cell Imaging Multi-Mode Reader. The specific lysis was calculated from the data according to the formula: % specific lysis=100×(experimental release−spontaneous release)/(maximum release−spontaneous release);

FIG. 18A-B illustrate CLL-1 CAR-T cell candidate-mediated cell killing is CLL-1 antigen specific: (A) The expression levels of CLL-1 antigens on MOLM-14^{wt} and MOLM-14^{CLL-1KO} cell pools were detected by flow cytometry using anti-human CLL-1-APC antibody (clone: REA431). (B) Cytolytic activity of CLL-1 CAR-T cell candidate prepared from Donor S032 and Donor S040 against MOLM-14^{wt} and MOLM-14^{CLL-1KO} pools at different effector to target (E: T) ratios was measured using luciferase-based reporter assay;

FIG. 19A-B illustrate the minimum effective dose (MED) and persistent anti-tumor activities of CLL-1 CAR-T cells. Mice (n=6) receiving U937_Luc transplant at 3×10⁴ per mouse on day 0 were then infused with the indicated doses of CLL-1 CAR T cells at day 5. Tumor volume was assessed by BLI measurement across study. (A) The tumor burden of xenograft mice receiving different doses of CLL-1 CAR-T cells derived from donor #S011. At day 43, the surviving mice were re-challenged with the same initial amounts of U937-Luc cells. (B) Tumor burden of xenograft mice receiving CAR-T cells derived from donor #S012 and the subsequent tumor cell re-challenge;

FIG. 20A-B illustrate R/R AML patient-derived CLL-1 CAR-T cell-mediated cytotoxicity. R/R patient-derived CLL-CAR-T cells were incubated with autologous primary AML blasts at E/T ratios of 1, 2, 4 and 8 for 24 hrs. The absolute AML blasts (CD45dim CD34+CD38+) of each culture were counted by ratio comparison with counting beads by flow cytometry. (A) Pt-S008 CLL-1 CAR-T cytotoxicity. (B) Pt-S015 CLL-1 CAR-T cytotoxicity.

### REFERENCE TO AN ELECTRONIC SEQUENCE LISTING

The contents of the electronic sequence listing (131852-0001UT01_SL.xml; Size: 79.0 bytes; and Date of Creation: Nov. 27, 2023) is herein incorporated by reference in its entirety.

SEQUENCE LISTINGS
(GMCSFRss)
SEQ ID NO: 01
MLLLVTSLLLCELPHPAFLLIP
(linker)
SEQ ID NO: 02
GGGGSGGGGSGGGGS
(CD8hinge-CD8TM)
SEQ ID NO: 03
TTTPAPRPPTPAPTIASQPLSLRPEACRPAAGGAVHTRGLDFACDIYIWAPLAGTCGVLLLS
LVITLYC
(CD28hinge-CD28TM)
SEQ ID NO: 04
IEVMYPPPYLDNEKSNGTIIHVKGKHLCPSPLFPGPSKPFWVLVVVGGVLACYSLLVTVAF
IIFWV
(4-1BB co-stimulatory)
SEQ ID NO: 05
KRGRKKLLYIFKQPFMRPVQTTQEEDGCSCRFPEEEEGGCEL
(CD28 co-stimulatory)
SEQ ID NO: 06
RSKRSRLLHSDYMNMTPRRPGPTRKHYQPYAPPRDFAAYRS
(CD3zeta)
SEQ ID NO: 07
RVKFSRSADAPAYKQGQNQLYNELNLGRREEYDVLDKRRGRDPEMGGKPRRKNPQEGL
YNELQKDKMAEAYSEIGMKGERRRGKGHDGLYQGLSTATKDTYDALHMQALPPR
(T2A)
SEQ ID NO: 08
EGRGSLLTCGDVEENPGP
(Human IgG1 CH2CH3)
SEQ ID NO: 09
AAAPKSSDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLSLSPG
(anti-CLL-1-59A09_VH; CDRs are underlined)
SEQ ID NO: 10
EVQLVESGGGLVQPGGSLRLSCAASGFTFTDYYIHWVRQAPGKGLEWVSMISPSGGGEA
YYADSVKGRFTISRDNSKNTLYLQMNSLRAEDTAVYYCARAHDFDDFDYWGQGTLVTV
SS
(anti-CLL-1-59A09_VL; CDRs are underlined)
SEQ ID NO: 11
DIQMTQSPSSLSASVGDRVTITCRASQSIRYSLAWYQQKPGKASKLLIYDASTLQSGVPSR
FSGSGSGTDFTLTISSLQPEDFATYYCQQYNNSPLTFGQGTKVEIKR
(anti-CLL-1-61A07_VH; CDRs are underlined)
SEQ ID NO: 12
EVQLVESGGGLVQPGGSLRLSCAASGFTFSNHWMHWVRQAPGKGLEWVSSISGAGGGK
SYADSVKGRFTISRDNSKNTLYLQMNSLRAEDTAVYYCARDESSYAYDGDYWGQGTLV
TVSS
(anti-CLL-1-61A07_VL; CDRs are underlined)
SEQ ID NO: 13
DIQMTQSPSSLSASVGDRVTITCRANQSIRNYLNWYQQKPGKAPKLLIYAASTLQSGVPSR
FSGSGSGTDFTLTISSLQPEDFATYYCQQNTSSPSTFGQGTKVEIKR
(anti-CLL-1-61H08_VH; CDRs are underlined)
SEQ ID NO: 14
EVQLVESGGGLVQPGGSLRLSCAASGFTFDDYGMHWVRQAPGKGLEWVSGIWPSGGNK
EYADSVKGRFTISRDNSKNTLYLQMNSLRAEDTAVYYCARLLALTYDYFDYWGQGTLV
TVSS
(anti-CLL-1-61H08_VL; CDRs are underlined)
SEQ ID NO: 15
DIQMTQSPSSLSASVGDRVTITCRASQGISSYLAWYQQKPGKAPKLLIYDASTLQSGVPSR
FSGSGSGTDFTLTISSLQPEDFATYYCQQSYSTPFTFGQGTKVEIKR
(anti-CLL-1-65D01_VH; CDRs are underlined)
SEQ ID NO: 16
QVQLQQWGAGLLKPSETLSLTCAVYGGSFSGYYWSWIRQPPGKGLEWIGEINHSGSTNY
NPSLKSRVTISVDTSKNQFSLKLSSVTAADTAAYYCARSPLQWLDDAFDIWGQGTMVTV
SS
(anti-CLL-1-65D01_VL; CDRs are underlined)
SEQ ID NO: 17
QSVLTQPPSVSAAPGQRVTISCSGSSSNIGSNYVSWYQQLPGTAPKLLIYDNNERPSGIPDR
FSGSKSATSATLDITGLQTGDEADYFCGTWDNSPSTDWVFGGGTKVTVLG
(anti-CLL-1-72C10_VH)
SEQ ID NO: 18
EVQLVESGGGLVQPGGSLRLSCAASGFTFSVNAMHWVRQAPGKGLEWVSTISGSDGHKY
YADSVKSRFTISRDNSKNTLYLQMNSLRAEDTAVYYCARDVDSEIGDGYHEDIWGQGTL
VTVSS
(anti-CLL-1-72C10_VL)
SEQ ID NO: 19
DIQMTQSPSSLSASVGDRVTITCRASQSVTNALNWYQQKPGKAPKLLIYAASNLQSGVPS
RFSGSGSGTDFTLTISSLQPEDFATYYCQQTNNSPATFGQGTKVEIKR

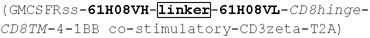

Amino acid sequence:
SEQ ID NO: 20
MLLLVTSLLLCELPHPAFLLIPEVQLVESGGGLVQPGGSLRLSCAASGFTFDDYGMHWVR
QAPGKGLEWVSGIWPSGGNKEYADSVKGRFTISRDNSKNTLYLQMNSLRAEDTAVYYC

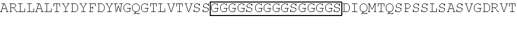

ITCRASQGISSYLAWYQQKPGKAPKLLIYDASTLQSGVPSRFSGSGSGTDFTLTISSLQPED
FATYYCQQSYSTPFTFGQGTKVEIKRTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCRF
PEEEEGGCELRVKFSRSADAPAYKQGQNQLYNELNLGRREEYDVLDKRRGRDPEMGGK
PRRKNPQEGLYNELQKDKMAEAYSEIGMKGERRRGKGHDGLYQGLSTATKDTYDALH
MQALPPREGRGSLLTCGDVEENPGP

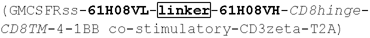

Amino acid sequence:
SEQ ID NO: 21
MLLLVTSLLLCELPHPAFLLIPDIQMTQSPSSLSASVGDRVTITCRASQGISSYLAWYQQKP
GKAPKLLIYDASTLQSGVPSRFSGSGSGTDFTLTISSLQPEDFATYYCQQSYSTPFTFGQGT

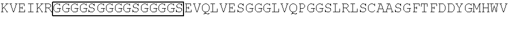

RQAPGKGLEWVSGIWPSGGNKEYADSVKGRFTISRDNSKNTLYLQMNSLRAEDTAVYYC
ARLLALTYDYFDYWGQGTLVTVSSTTTPAPRPPTPAPTIASQPLSLRPEACRPAAGGAVHTR
GLDFACDIYIWAPLAGTCGVLLLSLVITLYCKRGRKKLLYIFKQPFMRPVQTTQEEDGCSCRF
PEEEEGGCELRVKFSRSADAPAYKQGQNQLYNELNLGRREEYDVLDKRRGRDPEMGGK
PRRKNPQEGLYNELQKDKMAEAYSEIGMKGERRRGKGHDGLYQGLSTATKDTYDALH
MQALPPREGRGSLLTCGDVEENPGP

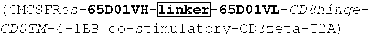

Amino acid sequence:
SEQ ID NO: 22
MLLLVTSLLLCELPHPAFLLIPQVQLQQWGAGLLKPSETLSLTCAVYGGSFSGYYWSWIR
QPPGKGLEWIGEINHSGSTNYNPSLKSRVTISVDTSKNQFSLKLSSVTAADTAAYYCARSP

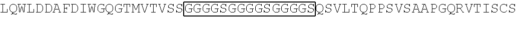

GSSSNIGSNYVSWYQQLPGTAPKLLIYDNNERPSGIPDRFSGSKSATSATLDITGLQTGDEA
DYFCGTWDNSPSTDWVFGGGTKVTVLGTTTPAPRPPTPAPTIASQPLSLRPEACRPAAGGAV
HTRGLDFACDIYIWAPLAGTCGVLLLSLVITLYCKRGRKKLLYIFKQPFMRPVQTTQEEDGCS
CRFPEEEEGGCELRVKFSRSADAPAYKQGQNQLYNELNLGRREEYDVLDKRRGRDPEMG
GKPRRKNPQEGLYNELQKDKMAEAYSEIGMKGERRRGKGHDGLYQGLSTATKDTYDAL
HMQALPPREGRGSLLTCGDVEENPGP

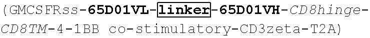

Amino acid sequence:
SEQ ID NO: 23
MLLLVTSLLLCELPHPAFLLIPQSVLTQPPSVSAAPGQRVTISCSGSSSNIGSNYVSWYQQL
PGTAPKLLIYDNNERPSGIPDRFSGSKSATSATLDITGLQTGDEADYFCGTWDNSPSTDWV

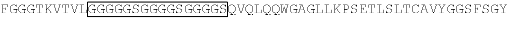

YWSWIRQPPGKGLEWIGEINHSGSTNYNPSLKSRVTISVDTSKNQFSLKLSSVTAADTAAY
YCARSPLQWLDDAFDIWGQGTMVTVSSTTTPAPRPPTPAPTIASQPLSLRPEACRPAAGGAV
HTRGLDFACDIYIWAPLAGTCGVLLLSLVITLYCKRGRKKLLYIFKQPFMRPVQTTQEEDGCS
CRFPEEEEGGCELRVKFSRSADAPAYKQGQNQLYNELNLGRREEYDVLDKRRGRDPEMG
GKPRRKNPQEGLYNELQKDKMAEAYSEIGMKGERRRGKGHDGLYQGLSTATKDTYDAL
HMQALPPREGRGSLLTCGDVEENPGP

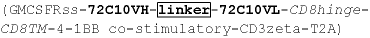

Amino acid sequence:
SEQ ID NO: 24
MLLLVTSLLLCELPHPAFLLIPEVQLVESGGGLVQPGGSLRLSCAASGFTFSVNAMHWVR
QAPGKGLEWVSTISGSDGHKYYADSVKSRFTISRDNSKNTLYLQMNSLRAEDTAVYYCA

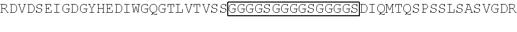

VTITCRASQSVTNALNWYQQKPGKAPKLLIYAASNLQSGVPSRFSGSGSGTDFTLTISSLQ
PEDFATYYCQQTNNSPATFGQGTKVEIKRTTTPAPRPPTPAPTIASQPLSLRPEACRPAAGGA
VHTRGLDFACDIYIWAPLAGTCGVLLLSLVITLYCKRGRKKLLYIFKQPFMRPVQTTQEEDGC
SCRFPEEEEGGCELRVKFSRSADAPAYKQGQNQLYNELNLGRREEYDVLDKRRGRDPEM
GGKPRRKNPQEGLYNELQKDKMAEAYSEIGMKGERRRGKGHDGLYQGLSTATKDTYDA
LHMQALPPREGRGSLLTCGDVEENPGP

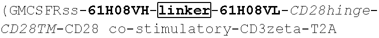

Amino acid sequence:
SEQ ID NO: 25
MLLLVTSLLLCELPHPAFLLIPEVQLVESGGGLVQPGGSLRLSCAASGFTFDDYGMHWVR
QAPGKGLEWVSGIWPSGGNKEYADSVKGRFTISRDNSKNTLYLQMNSLRAEDTAVYYC

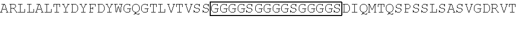

ITCRASQGISSYLAWYQQKPGKAPKLLIYDASTLQSGVPSRFSGSGSGTDFTLTISSLQPED
FATYYCQQSYSTPFTFGQGTKVEIKRIEVMYPPPYLDNEKSNGTIIHVKGKHLCPSPLFPGPS
KPFWVLVVVGGVLACYSLLVTVAFIIFWVRSKRSRLLHSDYMNMTPRRPGPTRKHYQPYAPP
RDFAAYRSRVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDTYDALHMQ
ALPPREGRGSLLTCGDVEENPGP

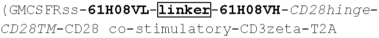

Amino acid sequence:
SEQ ID NO: 26
MLLLVTSLLLCELPHPAFLLIPDIQMTQSPSSLSASVGDRVTITCRASQGISSYLAWYQQKP
GKAPKLLIYDASTLQSGVPSRFSGSGSGTDFTLTISSLQPEDFATYYCQQSYSTPFTFGQGT

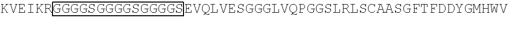

RQAPGKGLEWVSGIWPSGGNKEYADSVKGRFTISRDNSKNTLYLQMNSLRAEDTAVYYC
ARLLALTYDYFDYWGQGTLVTVSSIEVMYPPPYLDNEKSNGTIIHVKGKHLCPSPLFPGPSK
PFWVLVVVGGVLACYSLLVTVAFIIFWVRSKRSRLLHSDYMNMTPRRPGPTRKHYQPYAPPR
DFAAYRSRVKFSRSADAPAYKQGQNQLYNELNLGRREEYDVLDKRRGRDPEMGGKPRR
KNPQEGLYNELQKDKMAEAYSEIGMKGERRRGKGHDGLYQGLSTATKDTYDALHMQA
LPPREGRGSLLTCGDVEENPGP

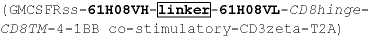

DNA sequence:
SEQ ID NO: 27
ATGCTGCTGCTGGTTACATCTCTGCTGCTGTGCGAGCTGCCCCATCCTGCCTTTCTGCT
GATCCCTGAAGTTCAGCTGGTTGAAAGCGGCGGTGGTCTGGTTCAGCCTGGTGGCAG
CCTGCGCCTGAGCTGTGCAGCAAGCGGCTTTACCTTTGATGATTACGGTATGCATTGG
GTTCGCCAGGCACCTGGCAAAGGCCTGGAATGGGTTAGCGGTATTTGGCCATCTGGT
GGTAACAAAGAATATGCAGATAGCGTTAAAGGCCGCTTTACCATTAGCCGCGATAAT
AGCAAAAATACCCTGTACCTGCAAATGAACAGCCTGCGCGCAGAGGATACCGCCGTG
TACTATTGTGCCCGCCTGCTGGCACTGACCTACGATTACTTTGATTACTGGGGCCAGG
GCACCCTGGTCACCGTGAGCTCTGGTGGAGGCGGTTCAGGCGGAGGTGGCTCTGGCG
GTGGCGGATCGGATATCCAGATGACACAGAGCCCTAGCAGCCTGTCTGCCAGCGTGG
GCGATCGCGTGACCATCACCTGTAGAGCCAGCCAGGGTATCTCTTCTTACCTGGCATG
GTATCAGCAGAAGCCTGGCAAAGCCCCTAAACTGCTGATCTATGATGCATCTACCCTG
CAGTCTGGCGTGCCAAGCCGCTTTTCTGGCAGCGGCTCTGGCACCGACTTCACCCTGA
CCATCTCTAGCCTGCAGCCTGAAGACTTCGCCACCTATTATTGCCAGCAGTCTTACTCT
ACCCCATTTACCTTTGGCCAGGGCACCAAAGTGGAAATCAAACGCACCACAACACCC
GCTCCTCGGCCTCCAACACCAGCTCCAACAATTGCCAGCCAGCCTCTGTCTCTGAGGC
CCGAAGCTTGTAGACCAGCTGCTGGCGGAGCCGTGCATACAAGAGGACTGGATTTCG
CCTGCGACATCTACATCTGGGCTCCTCTGGCTGGAACCTGTGGCGTGCTGTTGCTGAG
CCTGGTCATCACCCTGTACTGCAAGCGGGGCAGAAAGAAGCTGCTGTACATCTTTAA
GCAGCCCTTCATGCGGCCCGTGCAGACCACACAAGAGGAAGATGGCTGCTCCTGCAG
ATTCCCCGAGGAAGAAGAAGGCGGCTGCGAGCTGAGAGTGAAGTTCAGCAGATCCGC
CGACGCTCCCGCCTATAAGCAGGGACAGAACCAGCTGTACAACGAGCTGAACCTGGG
GAGAAGAGAAGAGTACGACGTGCTGGACAAGCGGAGAGGCAGAGATCCTGAGATGG
GCGGCAAGCCCAGACGGAAGAATCCTCAAGAGGGCCTGTATAATGAGCTGCAGAAA
GACAAGATGGCCGAGGCCTACAGCGAGATCGGAATGAAGGGCGAGCGCAGAAGAGG
CAAGGGACACGATGGACTGTACCAGGGCCTGAGCACCGCCACAAAGGATACCTATGA
CGCCCTGCACATGCAGGCCCTGCCTCCAAGAGAAGGCAGAGGCTCTCTGCTGACATG
CGGCGACGTGGAAGAGAACCCTGGACCT

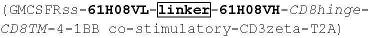

DNA sequence:
SEQ ID NO: 28
ATGCTGCTGCTGGTTACATCTCTGCTGCTGTGCGAGCTGCCCCATCCTGCCTTTCTGCT
GATCCCTGATATCCAGATGACACAGAGCCCTAGCAGCCTGTCTGCCAGCGTGGGCGA
TCGCGTGACCATCACCTGTAGAGCCAGCCAGGGTATCTCTTCTTACCTGGCATGGTAT
CAGCAGAAGCCTGGCAAAGCCCCTAAACTGCTGATCTATGATGCATCTACCCTGCAGT
CTGGCGTGCCAAGCCGCTTTTCTGGCAGCGGCTCTGGCACCGACTTCACCCTGACCAT
CTCTAGCCTGCAGCCTGAAGACTTCGCCACCTATTATTGCCAGCAGTCTTACTCTACC
CCATTTACCTTTGGCCAGGGCACCAAAGTGGAAATCAAACGCGGTGGAGGCGGTTCA
GGCGGAGGTGGCTCTGGCGGTGGCGGATCGGAAGTTCAGCTGGTTGAAAGCGGCGGT
GGTCTGGTTCAGCCTGGTGGCAGCCTGCGCCTGAGCTGTGCAGCAAGCGGCTTTACCT
TTGATGATTACGGTATGCATTGGGTTCGCCAGGCACCTGGCAAAGGCCTGGAATGGG
TTAGCGGTATTTGGCCATCTGGTGGTAACAAAGAATATGCAGATAGCGTTAAAGGCC
GCTTTACCATTAGCCGCGATAATAGCAAAAATACCCTGTACCTGCAAATGAACAGCCT
GCGCGCAGAGGATACCGCCGTGTACTATTGTGCCCGCCTGCTGGCACTGACCTACGAT
TACTTTGATTACTGGGGCCAGGGCACCCTGGTCACCGTGAGCTCTACCACAACACCCG
CTCCTCGGCCTCCAACACCAGCTCCAACAATTGCCAGCCAGCCTCTGTCTCTGAGGCC
CGAAGCTTGTAGACCAGCTGCTGGCGGAGCCGTGCATACAAGAGGACTGGATTTCGC
CTGCGACATCTACATCTGGGCTCCTCTGGCTGGAACCTGTGGCGTGCTGTTGCTGAGC
CTGGTCATCACCCTGTACTGCAAGCGGGGCAGAAAGAAGCTGCTGTACATCTTTAAG
CAGCCCTTCATGCGGCCCGTGCAGACCACACAAGAGGAAGATGGCTGCTCCTGCAGA
TTCCCCGAGGAAGAAGAAGGCGGCTGCGAGCTGAGAGTGAAGTTCAGCAGATCCGCC
GACGCTCCCGCCTATAAGCAGGGACAGAACCAGCTGTACAACGAGCTGAACCTGGGG
AGAAGAGAAGAGTACGACGTGCTGGACAAGCGGAGAGGCAGAGATCCTGAGATGGG
CGGCAAGCCCAGACGGAAGAATCCTCAAGAGGGCCTGTATAATGAGCTGCAGAAAG
ACAAGATGGCCGAGGCCTACAGCGAGATCGGAATGAAGGGCGAGCGCAGAAGAGGC
AAGGGACACGATGGACTGTACCAGGGCCTGAGCACCGCCACAAAGGATACCTATGAC
GCCCTGCACATGCAGGCCCTGCCTCCAAGAGAAGGCAGAGGCTCTCTGCTGACATGC
GGCGACGTGGAAGAGAACCCTGGACCT

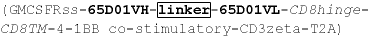

DNA sequence:
SEQ ID NO: 29
ATGCTGCTGCTGGTTACATCTCTGCTGCTGTGCGAGCTGCCCCATCCTGCCTTTCTGCT
GATCCCTCAGGTGCAGCTACAGCAGTGGGGCGCAGGACTGTTGAAGCCTTCGGAGAC
CCTGTCCCTCACCTGCGCTGTCTATGGTGGGTCCTTCAGTGGTTACTACTGGAGCTGG
ATCCGCCAGCCCCCAGGGAAGGGGCTGGAGTGGATTGGGGAAATCAATCATAGTGGA
AGCACCAACTACAACCCGTCCCTCAAGAGTCGAGTCACCATATCAGTAGACACGTCC
AAGAACCAGTTCTCCCTGAAGCTGAGCTCTGTGACCGCCGCGGACACGGCTGCGTATT
ACTGTGCGAGATCCCCCCTTCAGTGGCTGGATGATGCTTTTGATATCTGGGGCCAAGG
GACAATGGTCACCGTCTCTTCAGGTGGAGGCGGTTCAGGCGGAGGTGGCTCTGGCGG
TGGCGGATCGCAGTCTGTGTTGACGCAGCCGCCCTCAGTGTCTGCGGCCCCAGGACA
GAGGGTCACCATCTCCTGCTCTGGAAGCAGCTCCAACATTGGGAGTAATTATGTATCC
TGGTACCAGCAGCTCCCAGGAACAGCCCCCAAACTCCTCATTTATGACAATAATGAG
CGACCCTCAGGGATTCCTGACCGATTCTCTGGCTCCAAGTCTGCCACGTCAGCCACCC
TGGACATCACCGGACTCCAGACTGGGGACGAGGCCGATTATTTCTGCGGAACATGGG
ATAACAGCCCGAGTACTGATTGGGTGTTCGGCGGAGGGACCAAGGTGACCGTCCTAG
GTACCACAACACCCGCTCCTCGGCCTCCAACACCAGCTCCAACAATTGCCAGCCAGCC
TCTGTCTCTGAGGCCCGAAGCTTGTAGACCAGCTGCTGGCGGAGCCGTGCATACAAG
AGGACTGGATTTCGCCTGCGACATCTACATCTGGGCTCCTCTGGCTGGAACCTGTGGC
GTGCTGTTGCTGAGCCTGGTCATCACCCTGTACTGCAAGCGGGGCAGAAAGAAGCTG
CTGTACATCTTTAAGCAGCCCTTCATGCGGCCCGTGCAGACCACACAAGAGGAAGAT
GGCTGCTCCTGCAGATTCCCCGAGGAAGAAGAAGGCGGCTGCGAGCTGAGAGTGAAG
TTCAGCAGATCCGCCGACGCTCCCGCCTATAAGCAGGGACAGAACCAGCTGTACAAC
GAGCTGAACCTGGGGAGAAGAGAAGAGTACGACGTGCTGGACAAGCGGAGAGGCAG
AGATCCTGAGATGGGCGGCAAGCCCAGACGGAAGAATCCTCAAGAGGGCCTGTATAA
TGAGCTGCAGAAAGACAAGATGGCCGAGGCCTACAGCGAGATCGGAATGAAGGGCG
AGCGCAGAAGAGGCAAGGGACACGATGGACTGTACCAGGGCCTGAGCACCGCCACA
AAGGATACCTATGACGCCCTGCACATGCAGGCCCTGCCTCCAAGAGAAGGCAGAGGC
TCTCTGCTGACATGCGGCGACGTGGAAGAGAACCCTGGACCT

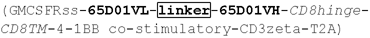

DNA sequence:
SEQ ID NO: 30
ATGCTGCTGCTGGTTACATCTCTGCTGCTGTGCGAGCTGCCCCATCCTGCCTTTCTGCT
GATCCCTCAGTCTGTGTTGACGCAGCCGCCCTCAGTGTCTGCGGCCCCAGGACAGAGG
GTCACCATCTCCTGCTCTGGAAGCAGCTCCAACATTGGGAGTAATTATGTATCCTGGT
ACCAGCAGCTCCCAGGAACAGCCCCCAAACTCCTCATTTATGACAATAATGAGCGAC
CCTCAGGGATTCCTGACCGATTCTCTGGCTCCAAGTCTGCCACGTCAGCCACCCTGGA
CATCACCGGACTCCAGACTGGGGACGAGGCCGATTATTTCTGCGGAACATGGGATAA
CAGCCCGAGTACTGATTGGGTGTTCGGCGGAGGGACCAAGGTGACCGTCCTAGGTGG
TGGAGGCGGTTCAGGCGGAGGTGGCTCTGGCGGTGGCGGATCGCAGGTGCAGCTACA
GCAGTGGGGCGCAGGACTGTTGAAGCCTTCGGAGACCCTGTCCCTCACCTGCGCTGTC
TATGGTGGGTCCTTCAGTGGTTACTACTGGAGCTGGATCCGCCAGCCCCCAGGGAAG
GGGCTGGAGTGGATTGGGGAAATCAATCATAGTGGAAGCACCAACTACAACCCGTCC
CTCAAGAGTCGAGTCACCATATCAGTAGACACGTCCAAGAACCAGTTCTCCCTGAAG
CTGAGCTCTGTGACCGCCGCGGACACGGCTGCGTATTACTGTGCGAGATCCCCCCTTC
AGTGGCTGGATGATGCTTTTGATATCTGGGGCCAAGGGACAATGGTCACCGTCTCTTC
AACCACAACACCCGCTCCTCGGCCTCCAACACCAGCTCCAACAATTGCCAGCCAGCCT
CTGTCTCTGAGGCCCGAAGCTTGTAGACCAGCTGCTGGCGGAGCCGTGCATACAAGA
GGACTGGATTTCGCCTGCGACATCTACATCTGGGCTCCTCTGGCTGGAACCTGTGGCG
TGCTGTTGCTGAGCCTGGTCATCACCCTGTACTGCAAGCGGGGCAGAAAGAAGCTGC
TGTACATCTTTAAGCAGCCCTTCATGCGGCCCGTGCAGACCACACAAGAGGAAGATG
GCTGCTCCTGCAGATTCCCCGAGGAAGAAGAAGGCGGCTGCGAGCTGAGAGTGAAGT
TCAGCAGATCCGCCGACGCTCCCGCCTATAAGCAGGGACAGAACCAGCTGTACAACG
AGCTGAACCTGGGGAGAAGAGAAGAGTACGACGTGCTGGACAAGCGGAGAGGCAGA
GATCCTGAGATGGGCGGCAAGCCCAGACGGAAGAATCCTCAAGAGGGCCTGTATAAT
GAGCTGCAGAAAGACAAGATGGCCGAGGCCTACAGCGAGATCGGAATGAAGGGCGA
GCGCAGAAGAGGCAAGGGACACGATGGACTGTACCAGGGCCTGAGCACCGCCACAA
AGGATACCTATGACGCCCTGCACATGCAGGCCCTGCCTCCAAGAGAAGGCAGAGGCT
CTCTGCTGACATGCGGCGACGTGGAAGAGAACCCTGGACCT

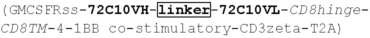

DNA sequence:
SEQ ID NO: 31
ATGCTGCTGCTGGTTACATCTCTGCTGCTGTGCGAGCTGCCCCATCCTGCCTTTCTGCT
GATCCCTGAAGTTCAGCTGGTTGAAAGCGGCGGTGGTCTGGTTCAGCCTGGTGGCAG
CCTGCGCCTGAGCTGTGCAGCAAGCGGCTTTACCTTTTCTGTTAACGCAATGCATTGG
GTTCGCCAGGCACCTGGCAAAGGCCTGGAATGGGTTAGCACAATTTCTGGTTCTGATG
GTCATAAATACTATGCAGATAGCGTTAAAAGCCGCTTTACCATTAGCCGCGATAATAG
CAAAAATACCCTGTACCTGCAAATGAACAGCCTGCGCGCAGAGGATACCGCCGTGTA
CTATTGTGCCCGCGATGTTGATTCTGAAATCGGTGATGGTTACCATGAAGATATCTGG
GGCCAGGGCACCCTGGTCACCGTGAGCTCTGGTGGAGGCGGTTCAGGCGGAGGTGGC
TCTGGCGGCGGCGGATCGGATATCCAGATGACACAGAGCCCTAGCAGCCTGTCTGCC
AGCGTGGGCGATCGCGTGACCATCACCTGTAGAGCCAGCCAGTCTGTTACCAACGCA
CTGAACTGGTATCAGCAGAAGCCTGGCAAAGCCCCTAAACTGCTGATCTATGCAGCA
TCTAACCTGCAGTCTGGCGTGCCAAGCCGCTTTTCTGGCAGCGGCTCTGGCACCGACT
TCACCCTGACCATCTCTAGCCTGCAGCCTGAAGACTTCGCCACCTATTATTGCCAGCA
GACCAACAACTCTCCAGCAACCTTTGGCCAGGGCACCAAAGTGGAAATCAAACGCAC
CACAACACCCGCTCCTCGGCCTCCAACACCAGCTCCAACAATTGCCAGCCAGCCTCTG
TCTCTGAGGCCCGAAGCTTGTAGACCAGCTGCTGGCGGAGCCGTGCATACAAGAGGA
CTGGATTTCGCCTGCGACATCTACATCTGGGCTCCTCTGGCTGGAACCTGTGGCGTGC
TGTTGCTGAGCCTGGTCATCACCCTGTACTGCAAGCGGGGCAGAAAGAAGCTGCTGT
ACATCTTTAAGCAGCCCTTCATGCGGCCCGTGCAGACCACACAAGAGGAAGATGGCT
GCTCCTGCAGATTCCCCGAGGAAGAAGAAGGCGGCTGCGAGCTGAGAGTGAAGTTCA
GCAGATCCGCCGACGCTCCCGCCTATAAGCAGGGACAGAACCAGCTGTACAACGAGC
TGAACCTGGGGAGAAGAGAAGAGTACGACGTGCTGGACAAGCGGAGAGGCAGAGAT
CCTGAGATGGGCGGCAAGCCCAGACGGAAGAATCCTCAAGAGGGCCTGTATAATGAG
CTGCAGAAAGACAAGATGGCCGAGGCCTACAGCGAGATCGGAATGAAGGGCGAGCG
CAGAAGAGGCAAGGGACACGATGGACTGTACCAGGGCCTGAGCACCGCCACAAAGG
ATACCTATGACGCCCTGCACATGCAGGCCCTGCCTCCAAGAGAAGGCAGAGGCTCTC
TGCTGACATGCGGCGACGTGGAAGAGAACCCTGGACCT

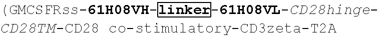

DNA sequence:
SEQ ID NO: 32
ATGCTGCTGCTGGTTACATCTCTGCTGCTGTGCGAGCTGCCCCATCCTGCCTTTCTGCT
GATCCCTGAAGTTCAGCTGGTTGAAAGCGGCGGTGGTCTGGTTCAGCCTGGTGGCAG
CCTGCGCCTGAGCTGTGCAGCAAGCGGCTTTACCTTTGATGATTACGGTATGCATTGG
GTTCGCCAGGCACCTGGCAAAGGCCTGGAATGGGTTAGCGGTATTTGGCCATCTGGT
GGTAACAAAGAATATGCAGATAGCGTTAAAGGCCGCTTTACCATTAGCCGCGATAAT
AGCAAAAATACCCTGTACCTGCAAATGAACAGCCTGCGCGCAGAGGATACCGCCGTG
TACTATTGTGCCCGCCTGCTGGCACTGACCTACGATTACTTTGATTACTGGGGCCAGG
GCACCCTGGTCACCGTGAGCTCTGGTGGAGGCGGTTCAGGCGGAGGTGGCTCTGGCG
GTGGCGGATCGGATATCCAGATGACACAGAGCCCTAGCAGCCTGTCTGCCAGCGTGG
GCGATCGCGTGACCATCACCTGTAGAGCCAGCCAGGGTATCTCTTCTTACCTGGCATG
GTATCAGCAGAAGCCTGGCAAAGCCCCTAAACTGCTGATCTATGATGCATCTACCCTG
CAGTCTGGCGTGCCAAGCCGCTTTTCTGGCAGCGGCTCTGGCACCGACTTCACCCTGA
CCATCTCTAGCCTGCAGCCTGAAGACTTCGCCACCTATTATTGCCAGCAGTCTTACTCT
ACCCCATTTACCTTTGGCCAGGGCACCAAAGTGGAAATCAAACGCATTGAAGTTATGT
ATCCTCCTCCTTACCTAGACAATGAGAAGAGCAATGGAACCATTATCCATGTGAAAG
GGAAACACCTTTGTCCAAGTCCCCTATTTCCCGGACCTTCTAAGCCCTTTTGGGTGCTG
GTGGTGGTTGGTGGAGTCCTGGCTTGCTATAGCTTGCTAGTAACAGTGGCCTTTATTA
TTTTCTGGGTGAGGAGTAAGAGGAGCAGGCTCCTGCACAGTGACTACATGAACATGA
CTCCCCGCCGCCCCGGGCCCACCCGCAAGCATTACCAGCCCTATGCCCCACCACGCGA
CTTCGCAGCCTATCGCTCCAGAGTGAAGTTCAGCAGATCCGCCGACGCTCCCGCCTAT
AAGCAGGGACAGAACCAGCTGTACAACGAGCTGAACCTGGGGAGAAGAGAAGAGTA
CGACGTGCTGGACAAGCGGAGAGGCAGAGATCCTGAGATGGGCGGCAAGCCCAGAC
GGAAGAATCCTCAAGAGGGCCTGTATAATGAGCTGCAGAAAGACAAGATGGCCGAG
GCCTACAGCGAGATCGGAATGAAGGGCGAGCGCAGAAGAGGCAAGGGACACGATGG
ACTGTACCAGGGCCTGAGCACCGCCACAAAGGATACCTATGACGCCCTGCACATGCA
GGCCCTGCCTCCAAGAGAAGGCAGAGGCTCTCTGCTGACATGCGGCGACGTGGAAGA
GAACCCTGGACCT

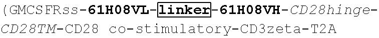

DNA sequence:
SEQ ID NO: 33
ATGCTGCTGCTGGTTACATCTCTGCTGCTGTGCGAGCTGCCCCATCCTGCCTTTCTGCT
GATCCCTGATATCCAGATGACACAGAGCCCTAGCAGCCTGTCTGCCAGCGTGGGCGA
TCGCGTGACCATCACCTGTAGAGCCAGCCAGGGTATCTCTTCTTACCTGGCATGGTAT
CAGCAGAAGCCTGGCAAAGCCCCTAAACTGCTGATCTATGATGCATCTACCCTGCAGT
CTGGCGTGCCAAGCCGCTTTTCTGGCAGCGGCTCTGGCACCGACTTCACCCTGACCAT
CTCTAGCCTGCAGCCTGAAGACTTCGCCACCTATTATTGCCAGCAGTCTTACTCTACC
CCATTTACCTTTGGCCAGGGCACCAAAGTGGAAATCAAACGCGGTGGAGGCGGTTCA
GGCGGAGGTGGCTCTGGCGGTGGCGGATCGGAAGTTCAGCTGGTTGAAAGCGGCGGT
GGTCTGGTTCAGCCTGGTGGCAGCCTGCGCCTGAGCTGTGCAGCAAGCGGCTTTACCT
TTGATGATTACGGTATGCATTGGGTTCGCCAGGCACCTGGCAAAGGCCTGGAATGGG
TTAGCGGTATTTGGCCATCTGGTGGTAACAAAGAATATGCAGATAGCGTTAAAGGCC
GCTTTACCATTAGCCGCGATAATAGCAAAAATACCCTGTACCTGCAAATGAACAGCCT
GCGCGCAGAGGATACCGCCGTGTACTATTGTGCCCGCCTGCTGGCACTGACCTACGAT
TACTTTGATTACTGGGGCCAGGGCACCCTGGTCACCGTGAGCTCTATTGAAGTTATGT
ATCCTCCTCCTTACCTAGACAATGAGAAGAGCAATGGAACCATTATCCATGTGAAAG
GGAAACACCTTTGTCCAAGTCCCCTATTTCCCGGACCTTCTAAGCCCTTTTGGGTGCTG
GTGGTGGTTGGTGGAGTCCTGGCTTGCTATAGCTTGCTAGTAACAGTGGCCTTTATTA
TTTTCTGGGTGAGGAGTAAGAGGAGCAGGCTCCTGCACAGTGACTACATGAACATGA
CTCCCCGCCGCCCCGGGCCCACCCGCAAGCATTACCAGCCCTATGCCCCACCACGCGA
CTTCGCAGCCTATCGCTCCAGAGTGAAGTTCAGCAGATCCGCCGACGCTCCCGCCTAT
AAGCAGGGACAGAACCAGCTGTACAACGAGCTGAACCTGGGGAGAAGAGAAGAGTA
CGACGTGCTGGACAAGCGGAGAGGCAGAGATCCTGAGATGGGCGGCAAGCCCAGAC
GGAAGAATCCTCAAGAGGGCCTGTATAATGAGCTGCAGAAAGACAAGATGGCCGAG
GCCTACAGCGAGATCGGAATGAAGGGCGAGCGCAGAAGAGGCAAGGGACACGATGG
ACTGTACCAGGGCCTGAGCACCGCCACAAAGGATACCTATGACGCCCTGCACATGCA
GGCCCTGCCTCCAAGAGAAGGCAGAGGCTCTCTGCTGACATGCGGCGACGTGGAAGA
GAACCCTGGACCT

### DETAILED DESCRIPTION OF DISCLOSURE

Unless specifically defined herein, all technical and scientific terms used have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The present disclosure recognizes that adoptive immunotherapy, which involves the transfer of antigen-specific T cells generated ex vivo, is a promising strategy to treat viral infections and cancer. The T cells used for adoptive immunotherapy can be generated either by expansion of antigen-specific T cells or redirection of T cells through genetic engineering (Park, Rosenberg et al. 2011). Transfer of viral antigen specific T cells is a well-established procedure used for the treatment of transplant associated viral infections and rare viral-related malignancies. Similarly, isolation and transfer of tumor specific T cells has been shown to be successful in treating melanoma.

The present disclosure recognizes that novel specificities in T cells can been successfully generated through the genetic transfer of transgenic chimeric antigen receptors (scCARs) (Jena, Dotti et al. 2010). scCARs are synthetic receptors consisting of a targeting moiety that is associated with one or more signaling domains in a single fusion molecule. In general, the binding moiety of a scCAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation scCARs are derived from the cytoplasmic/intracellular region of the CD3zeta or the Fc receptor gamma chains. First generation scCARs have been shown to successfully redirect T cell cytotoxicity, however, they failed to provide prolonged expansion and anti-tumor activity in vivo. Signaling domains from co-stimulatory molecules including CD28, OX-40 (CD134), and 4-1BB (CD137) have been added alone (second generation) or in combination (third generation) to enhance survival and increase proliferation of scCAR modified T cells. scCARs have successfully allowed T cells to be redirected against antigens expressed at the surface of tumor cells from various malignancies including lymphomas and solid tumors (Jena, Dotti et al. 2010).

The present disclosure recognizes that induction treatments for acute myeloid leukemia (AML) have remained largely unchanged over the years and AML remains a disease of poor prognosis. AML is a disease characterized by the rapid proliferation of immature myeloid cells in the bone marrow resulting in dysfunctional hematopoiesis. Although standard induction chemotherapy can induce complete remissions, many patients eventually relapse and succumb to the disease, calling for the development of novel therapeutics for AML. Recent advances in the immunophenotyping of AML cells have revealed several AML associated cell surface antigens that may act as targets for future therapies.

The present disclosure recognizes CLL-1 (C-Type Lectin-Like Molecule-1) as an interesting tumoral antigen target as it is expressed by leukemic blasts at diagnosis from 85-92% of AML patients analyzed. It belongs to the group V C-type lectin-like receptor family and has molecular weight of 75 kDa. Group V molecules have a lectin-like domain that binds to non-sugar ligands. CLL-1 is a 265 amino acid type II transmembrane glycoprotein (Uniprot database: Q5QGZ9 for human protein encoded by gene n^{o} 160364 in “Entrez Gene” database) that contains a 200 AA extracellular domain. CLL-1 is also referred to in the literature and databases as MICL, CLEC12 and KLRL1.

The present disclosure recognizes that CLL-1 is a cell surface protein that is specifically expressed on most malignant lymphoid stem cells (AML LSC), while not being expressed on normal HSC (Van Rhenen et al, 2007). Meanwhile, CLL-1 was revealed to be a diagnostic marker in AML (Larsen et al, 2012). Anti-CLL-1 antibodies enable both AML-specific stem-cell detection and possibly antigen-targeting as distinguishing malignant cells from normal stem cells both at diagnosis and in remission (van Rhenen et al, 2007).

The present disclosure recognizes that monoclonal antibodies have often been used to treat lymphomas, but their use in leukemias has been more limited. Gemtuzumab ozogamicin (Mylotarg®) is a monoclonal antibody with a cell poison attached to it. Previously approved to treat AML in older patients, it was withdrawn from the market after studies found some toxicity associated with the product (press release of Dec. 10, 2010 in PMLIVE “ASH: Pfizer eyes re-launch of Mylotarg”). Other monoclonal therapeutic antibodies have shown adverse effects over the last decade (Klastersky, J. (2006) “Adverse effects of the humanized antibodies used as cancer therapeutics” Current Opinion in Oncology. 18 (4): 316-320)

The present disclosure recognizes that a new approach to target CCL-1 using immune cells endowed with specific chimeric antigen receptors based on anti-CLL-1 monoclonal antibodies, which redirect immune cell specificity towards CLL-1 positive cells. In addition, the present disclosure recognizes that the engineered immune cells that they obtained using this approach have proven efficacy to eliminate CLL-1 positive malignant cells.

The present disclosure opens the way to treating patients affected with a condition characterized by an overabundance of CLL-1-expressing cells using adoptive immunotherapy. Even more, the present invention provides with engineered allogeneic immune cells that may be used as “off-the-shelf” allogeneic therapeutic products.

The practice of some methods disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

### Certain Definitions

As used in the specification and claims, the singular forms “a,” “an,” and “the” include plural references unless the context clearly dictates otherwise. For example, the term “an antigen binding domain” includes a plurality of antigen binding domains.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g. a cell can be a synthetically made, sometimes termed an artificial cell).

The term “activation” and its grammatical equivalents as used herein can refer to a process whereby a cell transitions from a resting state to an active state. This process can comprise a response to an antigen, migration, and/or a phenotypic or genetic change to a functionally active state. For example, the term “activation” can refer to the stepwise process of T cell activation. In some cases, a T cell can require at least two signals to become fully activated.

The term “antigen,” as used herein, refers to a molecule or a fragment thereof capable of being bound by a selective binding agent. As an example, an antigen can be a ligand that can be bound by a selective binding agent such as a receptor. In some cases, the receptor may function as the antigen and the ligand may function as the selective binding agent. As another example, an antigen can be an antigenic molecule that can be bound by a selective binding agent such as an immunological protein (e.g., an antibody). In some cases, the immunological protein may serve as the antigen and the antigenic molecule may serve as the selective binding agent. An antigen can also refer to a molecule or fragment thereof capable of being used in an animal to produce antibodies capable of binding to that antigen.

The term “epitope” and its grammatical equivalents, as used herein, can refer to a part of an antigen that can be recognized by an antigen binding domain. Antigen binding domains can comprise, for example, proteins (e.g., antibodies, antibody fragments) present on a surface, for example a cell surface (e.g., B cells, T cells, CAR-T cells, or engineered cells).

The term “antibody,” as used herein, refers to a proteinaceous binding molecule with immunoglobulin-like functions. The term antibody includes antibodies (e.g., monoclonal and polyclonal antibodies), as well as derivatives, variants, and fragments thereof. Antibodies include, but are not limited to, immunoglobulins (Ig's) of different classes (i.e. IgA, IgG, IgM, IgD and IgE) and subclasses (such as IgG1, IgG2, etc.). A derivative, variant or fragment thereof can refer to a functional derivative or fragment which retains the binding specificity (e.g., complete and/or partial) of the corresponding antibody. Antigen-binding fragments include Fab, Fab′, F(ab′)₂, variable fragment (Fv), single chain variable fragment (scFv), minibodies, diabodies, and single-domain antibodies (“sdAb” or “nanobodies” or “camelids”). The term antibody includes antibodies and antigen-binding fragments of antibodies that have been optimized, engineered or chemically conjugated. Examples of antibodies that have been optimized include affinity-matured antibodies. Examples of antibodies that have been engineered include Fc optimized antibodies (e.g., antibodies optimized in the fragment crystallizable region) and multispecific antibodies (e.g., bispecific antibodies).

The term “antigen binding domain,” as used herein, refers to a protein or fragment thereof capable of binding an antigen or an epitope. As an example, an antigen binding domain can be a cellular receptor. As an example, an antigen binding domain can be an engineered cellular receptor. As an example, an antigen binding domain can be a soluble receptor. In some cases, an antigen binding domain can be the ligand which is bound by the cellular receptor, the engineered cellular receptor, and/or the soluble receptor.

The term “autologous” and its grammatical equivalents, as used herein, can refer to origination from the same being. For example, an autologous sample (e.g., cells) can refer to a sample which is removed, processed, and then given back to the same subject (e.g., patient) at a later time. Autologous, with respect to a process, can be distinguished from an allogenic process in which the donor of a sample (e.g., cells) and the recipient of the sample are not the same subject.

The terms “cancer neo-antigen,” “neo-antigen,” and “neo-epitope” and their grammatical equivalents, as used herein, can refer to antigens that are not encoded in a normal, non-mutated host genome. A “neo-antigen” can, in some instances, represent either oncogenic viral proteins or abnormal proteins that arise as a consequence of somatic mutations. For example, a neo-antigen can arise by the disruption of cellular mechanisms through the activity of viral proteins. As another example, a neo-antigen can arise from exposure to a carcinogenic compound, which in some cases can lead to a somatic mutation. This somatic mutation can lead to the formation of a tumor/cancer.

The term “cytotoxicity,” as used herein, refers to an unintended or undesirable alteration in the normal state of a cell. The normal state of a cell may refer to a state that is manifested or exists prior to the cell's exposure to a cytotoxic composition, agent and/or condition. A cell that is in a normal state can be in homeostasis. An unintended or undesirable alteration in the normal state of a cell can be manifested in the form of, for example, cell death (e.g., programmed cell death), a decrease in replicative potential, a decrease in cellular integrity such as membrane integrity, a decrease in metabolic activity, a decrease in developmental capability, or any of the cytotoxic effects disclosed herein.

The phrases “reducing cytotoxicity” and “reduce cytotoxicity,” as used herein, refer to a reduction in degree or frequency of unintended or undesirable alterations in the normal state of a cell upon exposure to a cytotoxic composition, agent and/or condition. The phrase can refer to reducing the degree of cytotoxicity in an individual cell that is exposed to a cytotoxic composition, agent and/or condition, or to reducing the number of cells of a population that exhibit cytotoxicity when the population of cells is exposed to a cytotoxic composition, agent and/or condition.

The term “expression” refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell.

The terms “derivative,” “variant,” and “fragment,” when used herein with reference to a polypeptide, refers to a polypeptide related to a wild type polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a wild type polypeptide.

The term “percent (%) identity,” as used herein, refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment, for purposes of determining percent identity, can be achieved in various ways that are within the skill in the art, for instance, using publicly available computer software such as BLAST, ALIGN, or Megalign (DNASTAR) software. Percent identity of two sequences can be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

The terms “subject,” “individual,” and “patient” are used interchangeably herein to refer to a vertebrate, preferably a mammal such as a human. Mammals include, but are not limited to, murines, simians, humans, farm animals, sport animals, and pets. Tissues, cells and their progeny of a biological entity obtained in vivo or cultured in vitro are also encompassed.

The terms “treatment” and “treating,” as used herein, refer to an approach for obtaining beneficial or desired results including, but not limited to, a therapeutic benefit and/or a prophylactic benefit. For example, a treatment can comprise administering a system or cell population disclosed herein. A therapeutic benefit can refer to any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

A “therapeutic effect” may occur if there is a change in the condition being treated. The change may be positive or negative. For example, a ‘positive effect’ may correspond to an increase in the number of activated T-cells in a subject. In another example, a ‘negative effect’ may correspond to a decrease in the amount or size of a tumor in a subject. A “change” in the condition being treated, may refer to at least 1%, 2%, 3%, 4%, 5%, 6%, 7%, 8%, 9%, 10%, 25%, 50%, 75%, or 100% change in the condition. The change can be based on improvements in the severity of the treated condition in an individual, or on a difference in the frequency of improved conditions in populations of individuals with and without the administration of a therapy. Similarly, a method of the present disclosure may comprise administering to a subject an amount of cells that is “therapeutically effective”. The term “therapeutically effective” should be understood to have a definition corresponding to ‘having a therapeutic effect’.

The term “effective amount” or “therapeutically effective amount” refers to the quantity of a composition, for example a composition comprising immune cells such as lymphocytes (e.g., T lymphocytes and/or NK cells), that is sufficient to result in a desired activity upon administration to a subject in need thereof. The term “therapeutically effective” can refer to a quantity of a composition that is sufficient to delay the manifestation, arrest the progression, relieve or alleviate at least one symptom of a disorder treated by the methods of the present disclosure.

The term “TIL” or tumor infiltrating lymphocyte and its grammatical equivalents, as used herein, can refer to a cell isolated from a tumor. A TIL can be any cell found within a tumor. For example, a TIL can be a cell that has migrated to a tumor. A TIL can be a cell that has infiltrated a tumor. A TIL can be a T cell, B cell, monocyte, natural killer (NK) cell, or any combination thereof. A TIL can be a mixed population of cells. A population of TILs can comprise cells of different phenotypes, cells of different degrees of differentiation, cells of different lineages, or any combination thereof.

### CLL1 Single-Chain Specific Chimeric Antigen Receptors

The present invention relates to CLL1 specific chimeric antigen receptor comprising an extracellular ligand-binding domain specifically directed against one portion of the CLL1 antigen, a transmembrane domain and a signaling transducing domain.

By chimeric antigen receptor (CAR) is intended molecules that combine an extracellular binding domain directed against a component present on a target cell, for example an antibody-based specificity for a desired antigen (e.g., tumor antigen) with an immune cell receptor component to generate a chimeric protein that will transduce an activating or inhibitory signal toward cellular immune activity.

The present invention more particularly relates to a CLL-1 specific chimeric antigen receptor (anti-CLL-1 CAR) comprising at least: an extracellular anti-CLL-1 antigen binding-domain, a transmembrane domain, and a cytoplasmic/intracellular/intracellular signaling domain.

Preferably, the CLL-1 specific chimeric antigen receptor according to the invention further comprises a co-stimulatory domain, and more preferably a CD28 or a 4-1BB co-stimulatory domain as described for instance by Jena, B., G. Dotti, et al. (2010). It can also comprise a transmembrane domain which can be a Cd8α transmembrane domain, as well as an optional hinge.

The signal transducing domain or “cytoplasmic/intracellular signaling domain” of a CAR according to the present invention is responsible for intracellular signaling following the binding of extracellular ligand binding domain to the target resulting in the activation or inhibition of the immune cell and immune response. In other words, the signal transducing domain is responsible for the activation or inactivation of at least one of the normal effector functions of the immune cell in which the CAR is expressed. For example, the effector function of a T cell can be a cytolytic activity or helper activity including the secretion of cytokines. Thus, the term “cytoplasmic/intracellular signaling domain” refers to the portion of a protein which transduces the effector signal function signal and directs the cell to perform a specialized function.

The cytoplasmic/intracellular signaling domain, which is preferably from a human protein involved in signal transduction pathway(s), determines whether anti-CLL-1 CAR is a positive CAR (PCAR) or a negative CAR (NCAR) depending on the nature of the signaling. Respectively, the CAR is a PCAR when the signaling domain, such as CD3zeta from human TCR receptor, has the effect of stimulating the cellular immune activity of the immune cell when the extracellular ligand binding-domain is bound to CLL-1. Conversely, the anti-CLL-1 CAR is a NCAR or inhibitory CAR (iCAR) when the signaling domain has the effect of reducing the cellular immune activity, such as signaling domains of human immunoinhibitory receptors CTLA-4 and PD-1 (Federov et al., Sci Transl Med. 2013 Dec. 11; 5 (215): 215ra172). Preferred examples of signal transducing domain for use in a anti-CLL1 CAR can be the cytoplasmic/intracellular sequences of the T cell receptor and co-receptors that act in concert to initiate signal transduction following antigen receptor engagement, as well as any derivate or variant of these sequences and any synthetic sequence that has the same functional capability. Signal transduction domain comprises two distinct classes of cytoplasmic/intracellular signaling sequence, those that initiate antigen-dependent primary activation, and those that act in an antigen-independent manner to provide a secondary or co-stimulatory signal. Primary cytoplasmic/intracellular signaling sequence can comprise signaling motifs which are known as immunoreceptor tyrosine-based activation motifs of ITAMs. ITAMs are well defined signaling motifs found in the intracytoplasmic/intracellular tail of a variety of receptors that serve as binding sites for syk/zap70 class tyrosine kinases. Examples of ITAM used in the invention can include as non limiting examples those derived from TCRzeta, FcRgamma, FcRbeta, FcRepsilon, CD3gamma, CD3delta, CD3epsilon, CD5, CD22, CD79a, CD79b and CD66d. In a preferred embodiment, the signaling transducing domain of the anti-CLL1 CAR can comprise the CD3zeta signaling domain which has amino acid sequence with at least 70%, preferably at least 80%, more preferably at least 90%, 95% 97% or 99% or 100% sequence identity with the signaling transducing domain in amino acid sequence selected from the group consisting of SEQ ID NO: 20-26.

A co-stimulatory molecule is a cell surface molecule other than an antigen receptor or their ligands that is required for an efficient immune response. “Co-stimulatory ligand” refers to a molecule on an antigen presenting cell that specifically binds a cognate co-stimulatory molecule on a T-cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1BBL, OX40L, inducible co-stimulatory ligand (ICOS-L), intercellular adhesion molecule (ICAM, CD30L, CD40, CD70, CD83, HLA-G, MICA, MICB, HVEM, lymphotoxin beta receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-1BB, OX40, CD30, CD40, PD-1, ICOS, lymphocyte function-associated antigen-1 (LFA-1), CD2, CD7, LTGHT, NKG2C, B7-H3, a ligand that specifically binds with CD83. A “co-stimulatory molecule” refers to the cognate binding partner on a T-cell that specifically binds with a co-stimulatory ligand, thereby mediating a co-stimulatory response by the cell, such as, but not limited to proliferation. Co-stimulatory molecules include, but are not limited to an MHC class I molecule, BTLA and Toll ligand receptor. Examples of costimulatory molecules include CD27, CD28, CD8, 4-1BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like.

In a preferred embodiment, the co-stimulatory domain of the anti-CLL1 CAR of the present invention comprises a part of co-stimulatory signal molecule selected from the group consisting of fragment of 4-1BB (GenBank: AAA53133.) and CD28 (NP_006130.1). In particular the signal transduction domain of the anti-CLL1 CAR of the present invention comprises amino acid sequence which comprises at least 70%, preferably at least 80%, more preferably at least 90%, 95% 97% or 99% sequence identity with the co-stimulatory domain in the amino acid sequence selected from the group consisting of SEQ ID NO: 20-26.

An anti-CLL1 CAR according to the present invention generally further comprises a transmembrane domain (TM). The distinguishing features of appropriate transmembrane domains comprise the ability to be expressed at the surface of a cell, preferably in the present invention an immune cell, in particular lymphocyte cells or Natural killer (NK) cells, and to interact together for directing cellular response of immune cell against a predefined target cell. The transmembrane domain can be derived either from a natural or from a synthetic source. The transmembrane domain can be derived from any membrane-bound or transmembrane protein. As non-limiting examples, the transmembrane polypeptide can be a subunit of the T-cell receptor such as α, β, γ or ζ, polypeptide constituting CD3 complex, IL2 receptor p55 (α chain), p75 ((β chain) or γ chain, subunit chain of Fc receptors, in particular Fcγ receptor III or CD proteins. Alternatively the transmembrane domain can be synthetic and can comprise predominantly hydrophobic residues such as leucine and valine. In a preferred embodiment said transmembrane domain is derived from the human CD8 alpha chain (e.g. NP_001139345.1) The transmembrane domain can further comprise a hinge region between said extracellular ligand-binding domain and said transmembrane domain.

The term “hinge region” used herein generally means any oligo- or polypeptide that functions to link the transmembrane domain to the extracellular ligand-binding domain. In particular, hinge region are used to provide more flexibility and accessibility for the extracellular ligand-binding domain. A hinge region may comprise up to 300 amino acids, preferably 10 to 100 amino acids and most preferably 25 to 50 amino acids. Hinge region may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence, or may be an entirely synthetic hinge sequence. In a preferred embodiment said hinge domain comprises hinge polypeptides which display preferably at least 80%, more preferably at least 90%, 95% 97% or 99% sequence identity with the hinger domain in the amino acid sequence selected from the group consisting of SEQ ID NO: 20-26. According to one embodiment, the hinge can also be a human Ig (immunoglobulin) hinge, e.g., a PD-1 hinge, an IgG4 hinge.

According to a preferred embodiment, the anti-CLL-1 CAR according to the invention comprises a transmembrane domain more particularly selected from CD8 and/or CD28.

An anti-CLL-1 CAR according to the invention generally further comprises a transmembrane domain (TM) more particularly a TM derived from CD8 and/or CD28, and even more particularly showing identity with the polypeptides of SEQ ID NO. 6 or 7.

In a preferred embodiment, an anti-CLL-1 CAR according to the invention further comprises a TM domain showing at least 90%, 91%, 92%, 93%, 94%, 95%, 96%, 97%, 98% or 99% sequence identity with the TM domain in the amino acid sequence selected from the group consisting of SEQ ID NO: 20-26.

Downregulation or mutation of target antigens is commonly observed in cancer cells, creating antigen-loss escape variants. Thus, to offset tumor escape and render immune cell more specific to target, the CLL-1 specific anti-CLL-1 CAR according to the invention can comprise another extracellular ligand-binding domains, to simultaneously bind different elements in target thereby augmenting immune cell activation and function. In one embodiment, the extracellular ligand-binding domains can be placed in tandem on the same transmembrane polypeptide, and optionally can be separated by a linker. In another embodiment, said different extracellular ligand-binding domains can be placed on different transmembrane polypeptides composing the anti-CLL-1 CAR. In another embodiment, the present invention relates to a population of anti-CLL-1 CAR s comprising each one different extracellular ligand binding domains. In a particular, the present invention relates to a method of engineering immune cells comprising providing an immune cell and expressing at the surface of said cell a population of anti-CLL-1 CAR each one comprising different extracellular ligand binding domains. In another particular embodiment, the present invention relates to a method of engineering an immune cell comprising providing an immune cell and introducing into said cell polynucleotides encoding polypeptides composing a population of anti-CLL-1 CAR each one comprising different extracellular ligand binding domains. By population of anti-CLL1 CAR s, it is meant at least two, three, four, five, six or more anti-CLL-1 CAR s each one comprising different extracellular ligand binding domains. The different extracellular ligand binding domains according to the present invention can preferably simultaneously bind different elements in target thereby augmenting immune cell activation and function. The present invention also relates to an isolated immune cell which comprises a population of anti-CLL-1 CAR s each one comprising different extracellular ligand binding domains.

CLL-1 specific chimeric antigen receptors according to the invention can have different architectures, as they can be expressed, for instance, under a single-chain chimeric protein (scCAR) or under the form of several polypeptides (multi-chain) including at least one such chimeric protein. Such multi-chain CAR architectures are disclosed in WO2014/039523, which are herein incorporated by reference.

The present application discloses several anti-CLL-1 single chain CAR directed against CLL-1 antigen, which comprise as non-limiting example the amino acid sequences: SEQ ID NO: 20-26.

CLL-1 CAR of the present invention can also be “multi-chain CARs” as previously mentioned, which means that the extracellular binding domain and the signaling domains are preferably located on different polypeptide chains, whereas co-stimulatory domains may be located on the same or a third polypeptide. Such multi-chain CARs can be derived from FcεRI (Ravetch et al, 1989), by replacing the high affinity IgE binding domain of FcεRI alpha chain by an extracellular ligand-binding domain such as scFv, whereas the N and/or C-termini tails of FcεRI beta and/or gamma chains are fused to signal transducing domains and co-stimulatory domains respectively. The extracellular ligand binding domain has the role of redirecting T-cell specificity towards cell targets, while the signal transducing domains activate or reduce the immune cell response. The fact that the different polypeptides derive from the alpha, beta and gamma polypeptides from FcεRI are transmembrane polypeptides sitting in juxtamembrane position provides a more flexible architecture to CARs, improving specificity towards the targeted molecule and reducing background activation of immune cells as described in WO2014/039523.

### Extracellular Antigen-Binding Domain

The term “extracellular antigen-binding domain” as used herein is defined as an oligo- or polypeptide that is capable of binding a ligand. Preferably, the domain will be capable of interacting with a cell surface molecule. For example, the extracellular ligand-binding domain may be chosen to recognize a ligand that acts as a cell surface marker on target cells associated with a particular disease state. It can be for instance binding domains derived from a ligand, a receptor, human or mice antibodies or antigen recognition domains derived from camels or cartilaginous fish.

In a preferred embodiment, said extracellular ligand-binding domain comprises a single chain antibody fragment (scFv) comprising the light (VL) and the heavy (VH) variable fragment of a target antigen specific monoclonal anti CLL-1 antibody joined by a flexible linker. Said VL and/or VH preferably show at least 90%, 91%, 92%, 93%, 94%, 95%, 96%, 97%, 98% or 99% sequence identity with VH and VL domains of amino acid sequence of SEQ ID NO. 20 to 26.

By the term “recombinant antibody” as used herein, is meant an antibody or antibody fragment which is generated using recombinant DNA technology, such as, for example, an antibody or antibody fragment expressed by a bacteriophage, a yeast expression system or a mammalian cell expression system. The term should also be construed to mean an antibody or antibody fragment which has been generated by the synthesis of a DNA molecule encoding the antibody or antibody fragment and which DNA molecule expresses an antibody or antibody fragment protein, or an amino acid sequence specifying the antibody or antibody fragment, wherein the DNA or amino acid sequence has been obtained using recombinant or synthetic DNA or amino acid sequence technology which is available and well known in the art.

The present invention discloses a CLL-1 specific single-chain chimeric antigen receptor (anti-CLL-1 scCAR) as described above, wherein said extra cellular ligand binding-domain comprises VH and VL chains which are humanized in some embodiments.

By the term “human/humanized antibody” as used herein, is meant the polypeptides include a human/humanized heavy chain variable region and a human/humanized light chain variable region. For example, the polypeptides may include the framework (FR) regions of the light and heavy chain variable regions of a human antibody, while retaining substantially the antigen-binding specificity of a parental monoclonal antibody. The human/humanized heavy chain variable region and/or the human/humanized light chain variable region are at least about 87% human/humanized, at least about 90% human/humanized, at least about 95% human/humanized, at least about 98% human/humanized, or at least about 100% human/humanized, excluding the complementary-determining regions (CDRs). The antigen-binding polypeptides molecules may be derived from monoclonal antibody donors (e.g., mouse monoclonal antibody donors) and may include CDRs from the monoclonal antibodies (e.g., mouse monoclonal CDRs).

By the term “monoclonal antibody” as used herein, is meant antibody produced by a laboratory-grown cell clone, either of a hybridoma or a virus-transformed lymphocyte that is more abundant and uniform than natural antibody and is able to bind specifically to a single site on CLL1 antigen. They are monospecific antibodies that are made by identical immune cells that are all clones of a unique parent cell, in contrast to polyclonal antibodies which are made from several different immune cells. Monoclonal antibodies have monovalent affinity, in that they bind to the same epitope. Current methodology applied for humanization is according to Lefranc M P et al (Lefranc, M P, Ehrenmann F, Ginestoux C, Giudicelli V, Duroux P “Use of IMGT® databases and tools for antibody engineering and humanization”, Methods Mol Biol. 2012; 907:3-37). In these four alignments are indicated.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592, 106 and EP 519,596; Padlan, 1991, Molecular Immunology, 28 (4/5): 489-498; Studnicka et al., 1994, Protein Engineering, 7 (6): 805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13 (5): 353-60 (2000), Morea et al., Methods, 20 (3): 267-79 (2000), Baca et al., J. Biol. Chem., 272 (16): 10678-84 (1997), Roguska et al., Protein Eng., 9 (10): 895-904 (1996), Couto et al., Cancer Res., 55 (23 Supp): 5973s-5977s (1995), Couto et al., Cancer Res., 55 (8): 1717-22 (1995), Sandhu J S, Gene, 150 (2): 409-10 (1994), and Pedersen et al., J. Mol. Biol., 235 (3): 959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.).

Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a anti-CLL-1 CAR of the invention can be replaced with other amino acid residues from the same side chain family and the altered anti-CLL-1 CAR can be tested for the ability to bind CLL-1 using the functional assays described herein.

In a preferred embodiment, the present invention discloses an anti-CLL-1 specific single-chain chimeric antigen receptor (“anti-CLL-1 scCAR” or “scCAR”) having a polypeptide structure selected from SEQ ID NO. 20-26, or polypeptide structure showing at least 90%, 91%, 92%, 93%, 94%, 95%, 96%, 97%, 98% or 99% sequence identity with amino acid sequence of SEQ ID NO. 20 to 26SEQ ID NO. 20-26. In some embodiments, said structure comprising an extra cellular antigen binding-domain comprising VH and VL from a monoclonal anti-CLL-1 antibody, a hinge, a transmembrane domain, a cytoplasmic/intracellular domain including a signaling domain and a co-stimulatory domain.

### Polynucleotides and Vectors

The present invention also relates to polynucleotides and vectors allowing heterologous expression into cells of the anti-CLL-1 CAR according to the invention, encoding the polypeptides sequences which have been previously detailed.

The polynucleotides may be included in an expression cassette or expression vector (e.g. a plasmid for introduction into a bacterial host cell, or a viral vector such as a baculovirus vector for transfection of an insect host cell, or a plasmid or viral vector such as a lentivirus for transfection of a mammalian host cell).

In a particular embodiment, the different nucleic acid sequences can be included in one polynucleotide or vector which comprises a nucleic acid sequence encoding ribosomal skip sequence such as a sequence encoding a 2A peptide. 2A peptides, which were identified in the Aphthovirus subgroup of picornaviruses, causes a ribosomal “skip” from one codon to the next without the formation of a peptide bond between the two amino acids encoded by the codons (see (Donnelly and Elliott 2001; Atkins, Wills et al. 2007; Doronina, Wu et al. 2008)). By “codon” is meant three nucleotides on an mRNA (or on the sense strand of a DNA molecule) that are translated by a ribosome into one amino acid residue. Thus, two polypeptides can be synthesized from a single, contiguous open reading frame within an mRNA when the polypeptides are separated by a 2A oligopeptide sequence that is in frame. Such ribosomal skip mechanisms are well known in the art and are known to be used by several vectors for the expression of several proteins encoded by a single messenger RNA.

To direct transmembrane polypeptide into the secretory pathway of a host cell, a secretory signal sequence (also known as a leader sequence, prepro sequence or pre sequence) is provided in polynucleotide sequence or vector sequence. The secretory signal sequence is operably linked to the transmembrane nucleic acid sequence, i.e., the two sequences are joined in the correct reading frame and positioned to direct the newly synthesized polypeptide into the secretory pathway of the host cell. Secretory signal sequences are commonly positioned 5′ to the nucleic acid sequence encoding the polypeptide of interest, although certain secretory signal sequences may be positioned elsewhere in the nucleic acid sequence of interest (see, e.g., Welch et al., U.S. Pat. No. 5,037,743; Holland et al., U.S. Pat. No. 5,143,830). In a preferred embodiment, the CAR polypeptide is derived from nucleic acid sequence SEQ ID NO: 27-33, or derived from nucleic acid showing at least 90%, 95% 97% or 99% sequence identity with SEQ ID NO: 27-33.

Those skilled in the art will recognize that, in view of the degeneracy of the genetic code, considerable sequence variation is possible among these polynucleotide molecules. Preferably, the nucleic acid sequences of the present invention are codon-optimized for expression in mammalian cells, preferably for expression in human cells. Codon-optimization refers to the exchange in a sequence of interest of codons that are generally rare in highly expressed genes of a given species by codons that are generally frequent in highly expressed genes of such species, such codons encoding the amino acids as the codons that are being exchanged.

### Delivery Methods

The present invention encompasses the different means to express the anti-CLL-1 Chimeric Antigen Receptor (CAR) described herein in immune cells

Methods for introducing a polynucleotide construct into cells are known in the art and include as non-limiting examples stable transformation methods wherein the polynucleotide construct encoding said CAR is integrated into the genome of the cell, transient transformation methods wherein the polynucleotide construct is not integrated into the genome of the cell and virus mediated methods.

Said polynucleotides may be introduced into a cell by for example, recombinant viral vectors (e.g. retroviruses, adenoviruses), liposome and the like. For example, transient transformation methods include for example microinjection, electroporation or particle bombardment, cell fusion. Said polynucleotides may be included in vectors, more particularly plasmids or virus, in view of being expressed in cells. Said plasmid vector can comprise a selection marker which provides for identification and/or selection of cells which received said vector.

Different transgenes can be included in one vector. Said vector can comprise a nucleic acid sequence encoding ribosomal skip sequence such as a sequence encoding a 2A peptide. 2A peptides, which were identified in the Aphthovirus subgroup of picornaviruses, causes a ribosomal “skip” from one codon to the next without the formation of a peptide bond between the two amino acids encoded by the codons (see Donnelly et al., J. of General Virology 82:1013-1025 (2001); Donnelly et al., J. of Gen. Virology 78:13-21 (1997); Doronina et al., Mol. And. Cell. Biology 28 (13): 4227-4239 (2008); Atkins et al., RNA 13:803-810 (2007)).

By “codon” is meant three nucleotides on an mRNA (or on the sense strand of a DNA molecule) that are translated by a ribosome into one amino acid residue. Thus, two polypeptides can be synthesized from a single, contiguous open reading frame within an mRNA when the polypeptides are separated by a 2A oligopeptide sequence that is in frame. Such ribosomal skip mechanisms are well known in the art and are known to be used by several vectors for the expression of several proteins encoded by a single messenger RNA.

In a more preferred embodiment of the invention, polynucleotides encoding polypeptides according to the present invention can be mRNA which is introduced directly into the cells, for example by electroporation. The inventors determined the optimal condition for mRNA electroporation in T-cell. The inventor used the cytoPulse technology which allows, by the use of pulsed electric fields, to transiently permeabilize living cells for delivery of material into the cells. The technology, based on the use of PulseAgile (BTX Havard Apparatus, 84 October Hill Road, Holliston, Mass. 01746, USA) electroporation waveforms grants the precise control of pulse duration, intensity as well as the interval between pulses (U.S. Pat. No. 6,010,613 and International PCT application WO2004083379). All these parameters can be modified in order to reach the best conditions for high transfection efficiency with minimal mortality. Basically, the first high electric field pulses allow pore formation, while subsequent lower electric field pulses allow moving the polynucleotide into the cell.

The different methods described above involve introducing scCAR into a cell. As non-limiting example, said scCAR can be introduced as transgenes encoded by one plasmid vector. Said plasmid vector can also contain a selection marker which provides for identification and/or selection of cells which received said vector.

Polypeptides may be synthesized in situ in the cell as a result of the introduction of polynucleotides encoding said polypeptides into the cell. Alternatively, said polypeptides could be produced outside the cell and then introduced thereto. Methods for introducing a polynucleotide construct into cells are known in the art and including as non-limiting examples stable transformation methods wherein the polynucleotide construct is integrated into the genome of the cell, transient transformation methods wherein the polynucleotide construct is not integrated into the genome of the cell and virus mediated methods. Said polynucleotides may be introduced into a cell by for example, recombinant viral vectors (e.g. retroviruses, adenoviruses), liposome and the like. For example, transient transformation methods include for example microinjection, electroporation or particle bombardment. Said polynucleotides may be included in vectors, more particularly plasmids or virus, in view of being expressed in cells.

### Activation and Expansion of T Cells

Whether prior to or after genetic modification of the T cells, even if the genetically modified immune cells of the present invention are activated and proliferate independently of antigen binding mechanisms, the immune cells, particularly T-cells of the present invention can be further activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. T cells can be expanded in vitro or in vivo.

Generally, the T cells of the invention are expanded by contact with an agent that stimulates a CD3 TCR complex and a co-stimulatory molecule on the surface of the T cells to create an activation signal for the T-cell. For example, chemicals such as calcium ionophore A23187, phorbol 12-myristate 13-acetate (PMA), or mitogenic lectins like phytohemagglutinin (PHA) can be used to create an activation signal for the T-cell.

As non-limiting examples, T cell populations may be stimulated in vitro such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, IL-4, IL-7, GM-CSF, -10, -2, 1L-15, TGFp, IL-21 and TNF- or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2). T cells that have been exposed to varied stimulation times may exhibit different characteristics

In another particular embodiment, said cells can be expanded by co-culturing with tissue or cells. Said cells can also be expanded in vivo, for example in the subject's blood after administrating said cell into the subject.

### Engineered Immune Cells

A “Cell” according to the present invention generally refers to a cell of hematopoietic origin functionally involved in the initiation and/or execution of innate and/or adaptive immune response. Cell according to the present invention is preferably an isolated immune cell, and more preferably a T-cell obtained from a donor. Said immune cell according to the present invention can also be derived from a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells. Representative human cells are CD34+ cells. Said isolated cell can also be a dendritic cell, killer dendritic cell, a mast cell, a NK-cell, a B-cell or a T-cell selected from the group consisting of inflammatory T-lymphocytes, cytotoxic T-lymphocytes, regulatory T-lymphocytes or helper T-lymphocytes. In another embodiment, said cell can be derived from the group consisting of CD4+ T-lymphocytes and CD8+ T-lymphocytes. Prior to expansion and genetic modification of the cells of the invention, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available and known to those skilled in the art, may be used.

In another embodiment, said cell can be derived from a healthy donor, from a patient diagnosed with cancer or from a patient diagnosed with an infection. In another embodiment, said cell is part of a mixed population of cells which present different phenotypic characteristics. In the scope of the present invention is also encompassed a cell line obtained from a transformed T-cell according to the method previously described. Modified cells resistant to an immunosuppressive treatment and susceptible to be obtained by the previous method are encompassed in the scope of the present invention.

As a preferred embodiment, the present invention provides T-cells or a population of primary T-cells, endowed with a CLL-1 CAR as described above, that do not express functional TCR and that a reactive towards CLL-1 positive cells, for their allogeneic transplantation into patients.

As a more preferred embodiment, the present invention provides T-cells or a population of T-cells endowed with a CLL-1 scCAR and that a reactive towards CLL-1 positive cells as described above, that do not express a functional TCR and are resistant to a selected drug, for their allogeneic transplantation into patients treated with said selected drug. The present invention encompasses the method of preparing engineered immune cells for immunotherapy comprising introducing ex-vivo into said immune cells the polynucleotides or vectors encoding the CLL-1 CAR according to transformation methods as previously described in WO2014/130635, WO2013176916, WO2013176915 and incorporated herein by reference.

In a preferred embodiment, said polynucleotides are introduced into the immune cells by means of retroviral vectors in view of being stably integrated into the cell genome.

Methods of Engineering Immune Cells with the CARs

The present invention also aims to produce immune cells endowed with anti CLL-1 CAR, which are less or non-alloreactive, which can be used in allogeneic treatments (i.e. with reduced risk of inducing Graft versus host reaction) and/or made resistant to various standard of care treatments).

As further described in this specification, said methods may further comprise the step of genetically modifying said immune cell by using at least one endonuclease.

The term “endonuclease” refers to any wild-type or variant enzyme capable of catalyzing the hydrolysis (cleavage) of bonds between nucleic acids within a DNA or RNA molecule, preferably a DNA molecule. Endonucleases do not cleave the DNA or RNA molecule irrespective of its sequence, but recognize and cleave the DNA or RNA molecule at specific polynucleotide sequences, further referred to as “target sequences” or “target sites”. Endonucleases can be classified as rare-cutting endonucleases when having typically a polynucleotide recognition site greater than 12 base pairs (bp) in length, more preferably of 14-55 bp.

Preferably, the methods according to the present invention involve a rare cutting endonuclease. Rare-cutting endonucleases can for example be a homing endonuclease (Paques and Duchateau 2007), a chimeric Zinc-Finger nuclease (ZFN) resulting from the fusion of engineered zinc-finger domains with the catalytic domain of a restriction enzyme such as FokI (Porteus and Carroll 2005), a TALE-nuclease, a Cas9 endonuclease from CRISPR system as described below (Gasiunas, Barrangou et al. 2012; Jinek, Chylinski et al. 2012; Cong, Ran et al. 2013; Mali, Yang et al. 2013) or a chemical endonuclease (Eisenschmidt, Lanio et al. 2005; Arimondo, Thomas et al. 2006). In chemical endonucleases, a chemical or peptidic cleaver is conjugated either to a polymer of nucleic acids or to another DNA recognizing a specific target sequence, thereby targeting the cleavage activity to a specific sequence. Chemical endonucleases also encompass synthetic nucleases like conjugates of orthophenanthroline, a DNA cleaving molecule, and triplex-forming oligonucleotides (TFOs), known to bind specific DNA sequences (Kalish and Glazer 2005). Rare-cutting endonucleases can be used for inactivating genes at a locus or to integrate transgenes by homologous recombination (HR) i.e. by inducing DNA double-strand breaks (DSBs) at a locus and insertion of exogeneous DNA at this locus by gene repair mechanism (Perrin, Buckle et al. 1993; Rouet, Smih et al. 1994; Choulika, Perrin et al. 1995; Pingoud and Silva 2007).

By “TALE-nuclease” (TALEN) is intended a fusion protein consisting of a nucleic acid-binding domain typically derived from a Transcription Activator Like Effector (TALE) and one nuclease catalytic domain to cleave a nucleic acid target sequence. The catalytic domain is preferably a nuclease domain and more preferably a domain having endonuclease activity, like for instance I-TevI, ColE7, NucA and Fok-I. In a particular embodiment, the TALE domain can be fused to a meganuclease like for instance I-CreI and 1-OnuI or functional variant thereof. In a more preferred embodiment, said nuclease is a monomeric TALE-Nuclease. A monomeric TALE-Nuclease is a TALE-Nuclease that does not require dimerization for specific recognition and cleavage, such as the fusions of engineered TAL repeats with the catalytic domain of 1-TevI described in WO2012138927. Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from new modular proteins recently discovered by the applicant in a different bacterial species. The new modular proteins have the advantage of displaying more sequence variability than TAL repeats. Preferably, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In another embodiment, critical amino acids 12 and 13 can be mutated towards other amino acid residues in order to modulate their specificity towards nucleotides A, T, C and G and in particular to enhance this specificity. TALE-nuclease have been already described and used to stimulate gene targeting and gene modifications (Boch, Scholze et al. 2009; Moscou and Bogdanove 2009; Christian, Cermak et al. 2010; Li, Huang et al. 2011). Engineered TAL-nucleases are available under the trade name TALEN™ (Cellectis, 8 rue de la Croix Jarry, 75013 Paris, France) and can be ordered from manufacturers, such as Life Technologies (Carlsbad, Calif., USA).

Preferred TALE-nucleases recognizing and cleaving the target sequence are described in PCT/EP2014/075317. In particular, additional catalytic domain can be further introduced into the cell with said rare-cutting endonuclease to increase mutagenesis in order to enhance their capacity to inactivate targeted genes. More particularly, said additional catalytic domain is a DNA end processing enzyme. Non limiting examples of DNA end-processing enzymes include 5-3′ -exonucleases, 3-5′ exonucleases, 5-3′ alkaline exonucleases, 5′ flap endonucleases, helicases, hosphatase, hydrolases and template-independent DNA polymerases. Non limiting examples of such catalytic domain comprise of a protein domain or catalytically active derivate of the protein domain selected from the group consisting of hExoI (EXO1_HUMAN), Yeast ExoI (EXO1_YEAST), E. coli ExoI, Human TREX2, Mouse TREX1, Human TREX1, Bovine TREX1, Rat TREX1, TdT (terminal deoxynucleotidyl transferase) Human DNA2, Yeast DNA2 (DNA2_YEAST). In a preferred embodiment, said additional catalytic domain has a 3′-5′-exonuclease activity, and in a more preferred embodiment, said additional catalytic domain is TREX, more preferably TREX2 catalytic domain (WO2012/058458). In another preferred embodiment, said catalytic domain is encoded by a single chain TREX2 polypeptide. Said additional catalytic domain may be fused to a nuclease fusion protein or chimeric protein according to the invention optionally by a peptide linker.

By “Cas9 endonuclease”. is meant any genome engineering tool developed based on the RNA-guided Cas9 nuclease (Gasiunas, Barrangou et al. 2012; Jinek, Chylinski et al. 2012; Cong, Ran et al. 2013; Mali, Yang et al. 2013) from the type II prokaryotic CRISPR (Clustered Regularly Interspaced Short palindromic Repeats) adaptive immune system (see for review (Sorek, Lawrence et al. 2013)). The CRISPR Associated (Cas) system was first discovered in bacteria and functions as a defense against foreign DNA, either viral or plasmid. CRISPR-mediated genome engineering first proceeds by the selection of target sequence often flanked by a short sequence motif, referred as the proto-spacer adjacent motif (PAM). Following target sequence selection, a specific crRNA, complementary to this target sequence is engineered. Trans-activating crRNA (tracrRNA) required in the CRISPR type II systems paired to the crRNA and bound to the provided Cas9 protein. Cas9 acts as a molecular anchor facilitating the base pairing of tracRNA with cRNA (Deltcheva, Chylinski et al. 2011). In this ternary complex, the dual tracrRNA: crRNA structure acts as guide RNA that directs the endonuclease Cas9 to the cognate target sequence. Target recognition by the Cas9-tracrRNA: crRNA complex is initiated by scanning the target sequence for homology between the target sequence and the crRNA. In addition to the target sequence-crRNA complementarity, DNA targeting requires the presence of a short motif adjacent to the protospacer (protospacer adjacent motif-PAM). Following pairing between the dual-RNA and the target sequence, Cas9 subsequently introduces a blunt double strand break 3 bases upstream of the PAM motif (Garneau, Dupuis et al. 2010). The use of Cas9 in immune cells, especially in T-Cells, has been previously described in WO2014191128.

In a preferred embodiment said method of further engineer the immune cells involves introducing into said T cells polynucleotides, in particular mRNAs, encoding specific rare-cutting endonuclease to selectively inactivate the genes mentioned above by DNA cleavage. In a more preferred embodiment said rare-cutting endonucleases are TALE-nucleases or Cas9 endonuclease. TAL-nucleases have so far proven higher specificity and cleavage efficiency over the other types of rare-cutting endonucleases, making them the endonucleases of choice for producing of the engineered immune cells on a large scale with a constant turn-over.

### Therapeutic Applications

In another embodiment, isolated cell obtained by the different methods or cell line derived from said isolated cell as previously described can be used as a medicament.

In another embodiment, said medicament can be used for treating cancer, particularly for the treatment of leukemia in a patient in need thereof.

In another embodiment, said isolated cell according to the invention or cell line derived from said isolated cell can be used in the manufacture of a medicament for treatment of a cancer in a patient in need thereof.

In a particular embodiment, an anti-CLL-1 CAR expressing T cell is provided as a medicament for the treatment of AML, of an AML subtype, of an AML-related complication, of an AML-related condition.

In another embodiment, said medicament can be used for treating a CLL-1-expressing cell-mediated pathological condition or a condition characterized by the direct or indirect activity of a CLL1-expressing cell.

In another aspect, the present invention relies on methods for treating patients in need thereof, said method comprising at least one of the following steps:
(a) providing an immune-cell obtainable by any one of the methods previously described;
(b) Administrating said transformed immune cells to said patient,

On one embodiment, said T cells of the invention can undergo robust in vivo T cell expansion and can persist for an extended amount of time.

Said treatment can be ameliorating, curative or prophylactic. It may be either part of an autologous immunotherapy or part of an allogenic immunotherapy treatment. By autologous, it is meant that cells, cell line or population of cells used for treating patients are originating from said patient or from a Human Leucocyte Antigen (HLA) compatible donor. By allogeneic is meant that the cells or population of cells used for treating patients are not originating from said patient but from a donor.

Cells that can be used with the disclosed methods are described in the previous section. Said treatment can be used to treat patients diagnosed wherein a pre-malignant or malignant cancer condition characterized by CLL-1-expressing cells, especially by an overabundance of CLL-1-expressing cells. Such conditions are found in hematologic cancers, such as leukemia.

In one embodiment, the present invention provides a composition for its use in the treatment of a CLL-1 expressing cells-mediated disease, in particular a CLL-1 expressing cells mediated hematologic cancer, said composition comprising said anti-CLL-1 scCAR expressing T cell of the invention.

Any other CLL-1-mediating or CLL-1-involving malignant lymphoproliferative disorders disclosed herein may be improved with the anti-CLL-1 CAR-expressing cells of the present invention.

In a preferred embodiment, the cancer that may be treated using the anti-CLL-1 CAR-expressing cells of the present invention is leukemia, a disease associated to leukemia or a complication thereof.

### AML

Leukemias that can be treated using the anti-CLL-1 CAR-expressing cells of the present invention can be acute myelogenous leukemia (AML). AML or AML subtypes that may be treated using the anti-CLL-1 scCAR-expressing cells of the present invention may be in particular, acute myeloblastic leukemia, minimally differentiated acute myeloblastic leukemia, acute myeloblastic leukemia without maturation, acute myeloblastic leukemia with granulocytic maturation, promyelocytic or acute promyelocytic leukemia (APL), acute myelomonocytic leukemia, myelomonocytic together with bone marrow eosinophilia, acute monoblastic leukemia (M5a) or acute monocytic leukemia (M5b), acute erythroid leukemias, including erythroleukemia (M6a) and very rare pure erythroid leukemia (M6b), acute megakaryoblastic leukemia, acute basophilic leukemia, acute panmyelosis with myelofibrosis, whether involving CLL-1-positive cells.

Subtypes of AML also include, hairy cell leukemia, philadelphia chromosome-positive acute lymphoblastic leukemia. AML may be classified as AML with specific genetic abnormalities. Classification is based on the ability of karyotype to predict response to induction therapy, relapse risk, survival.

Accordingly, AML that may be treated using the anti-CLL-1 CAR-expressing cells of the present invention may be AML with a translocation between chromosomes 8 and 21, AML with a translocation or inversion in chromosome 16, AML with a translocation between chromosomes 9 and 11, APL (M3) with a translocation between chromosomes 15 and 17, AML with a translocation between chromosomes 6 and 9, AML with a translocation or inversion in chromosome 3, AML (megakaryoblastic) with a translocation between chromosomes 1 and 22.

The present invention is particularly useful for the treatment of AML associated with these particular cytogenetic markers.

The present invention also provides an anti-CLL-1 CAR expressing T cell for the treatment of patients with specific cytogenetic subsets of AML, such as patients with t (15;17)(q22;q21) identified using all-trans retinoic acid (ATRA) 16-19 and for the treatment of patients with t(8;21)(q22;q22) or inv(16)(p13q22)/t(16;16)(p13;q22) identified using repetitive doses of high-dose cytarabine.

Preferably, the present invention provides an anti-CLL-1 CAR expressing T cell for the treatment of patients with aberrations, such as −5/del (5q), −7, abnormalities of 3q, or a complex karyotype, who have been shown to have inferior complete remission rates and survival.

The terms “therapeutic agent”, “chemotherapeutic agent”, or “drug” or “anti-cancer drug” as used herein refers to a medicament, preferably a compound or a derivative thereof that can interact with a cancer cell, thereby reducing the proliferative status of the cell and/or killing the cell. Examples of chemotherapeutic agents or “anti-cancer drug” include, but are not limited to, alkylating agents (e.g., busulfan, carboplatine, chlorambucil, cisplatine, cyclophosphamide, ifosfamide, melphalan, mechlorethamine, oxaliplatine, uramustine, temozolomide, fotemustine), metabolic antagonists (e.g., purine nucleoside antimetabolite such as clofarabine, methotrexate (MTX), 5-fluorouracil or derivatives thereof, azathioprine, capecitabine, cytarabine, floxuridine, fluorouracile, gemcitabine, methotrexate, pemetrexed), antitumor antibiotics (e.g., mitomycin, adriamycin, bleomycine, daunorubicine, doxorubicine, epirubicine, hydroxyurea, idarubicine, mitomycin C, mitoxantrone), plant-derived antitumor agents (e.g., vincristine, vindesine, taxol, vinblastine, vinorelbine, docetaxel, paclitaxel), topoisomerase inhibitor (irinotecan, topotecan, etoposide).

In a preferred embodiment, a therapeutic agent, a chemotherapy drug as used herein refers to a compound or a derivative thereof that may be used to treat cancer, in particular to treat a hematopoietic cancer cell and more particularly AML, thereby reducing the proliferative status of the cancer cell and/or killing the cancer cell. Examples of chemotherapeutic agents include, but are not limited to aracytine, Cytosine arabinoside, amsacrine, daunorubicine, idarubicine, novantrone, mitoxantrone, vepeside, etoposide (VP16), arsenic trioxyde, transretinoic acid, mechlorethamine, procarbazine, chlorambucil, and combination thereof.

In other embodiments of the present invention, cells of the invention are administered to a patient in conjunction with a drug (or an agent) selected from aracytine, cytosine arabinoside, amsacrine, daunorubicine, idarubicine, novantrone, mitoxantrone, vepeside, etoposide (VP16), arsenic trioxyde, transretinoic acid, cytarabine, anthracyclines, 6-thioguanine, hydroxyurea, prednisone, and combination thereof.

Such agents may further include, but are not limited to, the anti-cancer agents TRIMETHOTRIXATE™ (TMTX), TEMOZOLOMIDE™, RALTRITREXED™, S-(4-Nitrobenzyl)-6-thioinosine (NBMPR), 6-benzyguanidine (6-BG), bis-chloronitrosourea (BCNU) and CAMPTOTHECIN™, or a therapeutic derivative of any thereof.

In a more preferred embodiment an anti-CLL-1 scCAR expressing T cell, is administered to a patient, in combination with at least one therapeutic agent selected from aracytine, Cytosine arabinoside, amsacrine, daunorubicine, idarubicine, novantrone, mitoxantrone, vepeside, etoposide (VP16), arsenic trioxyde, transretinoic acid and combination thereof.

As used herein, a cell which is “resistant or tolerant” to an agent means a cell which has been genetically modified so that the cell proliferates in the presence of an amount of an agent that inhibits or prevents proliferation of a cell without the modification.

In one embodiment, the anti-CLL-1 CAR expressing T cell of present invention may be used as induction therapy, as post remission therapy of AML or as a consolidation therapy in patient with AML.

In one embodiment, the anti-CLL-1 CAR expressing T cell of the present invention may be used in case of AML relapse, or in case of refractory or resistant AML, and more preferably, in combination with at least one other anti-cancer drug

In another preferred embodiment, at least one anti-CLL-1 CAR expressing cell of the invention is used for preventing cancer cells development occurring in particular after anti-cancer treatment, during bone marrow depletion or before bone marrow transplantation, after bone marrow destruction.

### AML Complications

In one particular embodiment the invention provides a medicament that improves the health condition of a patient, in particular a patient undergoing a complication related to AML. More preferably, said engineered anti-CLL-1 CAR expressing T cell of the invention is expressing at least one anti-CLL-1 CAR of the invention and is used as a medicament for the treatment of a complication related to AML.

A complication or disease related to AML may include a preceding myelodysplasia phase, secondary leukemia, in particular secondary AML, high white blood cell count, and absence of Auer rods. Among others, leukostasis and involvement of the central nervous system (CNS), hyperleukocytosis, residual disease, are also considered as a complication or disease related to AML.

### AML Associated Diseases

In one embodiment, the present invention also provides an anti-CLL-1 CAR expressing T cell for the treatment of a pathological condition related to AML.

The present invention provides a therapy for AML related myeloid neoplasms, for acute myeloid leukemia and myelodysplastic syndrome, a treatment of relapsed or refractory acute myeloid leukemia, a treatment of relapsed or refractory acute promyelocytic leukemia in adults, a treatment for acute promyeloid leukaemia, a treatment of acute myeloid leukemia in adults over 60 years.

According to another aspect, the present invention provides a composition for the treatment of AML associated diseases, in particular hematologic malignancy related to AML.

Hematologic malignancy related to AML conditions include myelodysplasia syndromes (MDS, formerly known as “preleukemia”) which are a diverse collection of hematological conditions united by ineffective production (or dysplasia) of myeloid blood cells and risk of transformation to AML.

Other pathological conditions or genetic syndromes associated with the risk of AML can be improved with the adequate use of the present invention, said genetic syndromes include Down syndrome, trisomy, Fanconi anemia, Bloom syndrome, Ataxia-telangiectasia, Diamond-Blackfan anemia, Schwachman-Diamond syndrome, Li-Fraumeni syndrome, Neurofibromatosis type 1, Severe congenital neutropenia (also called Kostmann syndrome).

### Pharmaceutical Compositions and Method of Treatment

The present disclosure also provides a composition comprising an engineered T cells according to the disclosure for its use or a method for treating a disease.

In one aspect, the disease is a hematologic cancer, in particular a stem cell cancer including but is not limited to leukemia (such as acute myelogenous leukemia (AML) or a complication thereof.

The present disclosure also provides a composition for its use or a method for inhibiting the proliferation or reducing a CLL-1-expressing cell population or activity in a patient. An exemplary method includes contacting a population of cells comprising a CLL-1-expressing cell with an anti-CLL-1 CART cell, and in particular scCART, of the disclosure that binds to the CLL-1-expressing cell.

In a more specific aspect, the present disclosure provides a composition for its use or a method for inhibiting the proliferation or reducing the population of cancer cells expressing CLL-1 in a patient, the methods comprising contacting the CLL-1-expressing cancer cell population with an anti-CLL-1 CART cell, and in particular scCART, of the disclosure that binds to the CLL-1-expressing cell, binding of an anti-CLL-1 CAR cell, and in particular scCART, of the disclosure to the CLL-1-expressing cancer cell resulting in the destruction of the CLL-1-expressing cancer cells

In certain aspects, the anti-CLL-1 CART cell, and in particular scCART, of the disclosure reduces the quantity, number, amount or percentage of cells and/or cancer cells by at least 25%, at least 30%, at least 40%, at least 50%, at least 65%, at least 75%, at least 85%, at least 95%, or at least 99% (to undetectable level) in a subject with or animal model for myeloid leukemia or another cancer associated with CLL-1-expressing cells, relative to a negative control.

The present disclosure also provides a composition for its use or a method for preventing, treating and/or managing a disorder or condition associated with CLL-1-expressing cells (e.g., associated with a hematologic cancer), the methods comprising administering to a subject in need an anti-CLL-1 CART cell, and in particular scCART, of the disclosure that binds to the CLL-1-expressing cell. In one aspect, the subject is a human. Non-limiting examples of disorders associated with CLL-1-expressing cells include inflammatory disorders (such as rheumatoid arthritis) and cancers (such as hematological cancers, in particular AML or AML complications).

The present disclosure also provides a composition for its use or a method for preventing, treating and/or managing a disease associated with CLL-1-expressing cells, the method comprising administering to a subject in need an anti-CLL-1 CART cell, and in particular scCART, of the disclosure that binds to the CLL-1-expressing cell. In one aspect, the subject is a human. Non-limiting examples of diseases associated with CLL-1-expressing cells include in particular Acute Myeloid Leukemia (AML).

The present disclosure provides a composition for its use or a method for treating or preventing relapse of cancer associated with CLL-1-expressing cells, the method comprising administering to a subject in need thereof an anti-CLL-1 CART cell, and in particular scCART, of the disclosure that binds to the CLL-1-expressing cell. In another aspect, the methods comprise administering to the subject in need thereof an effective amount of an anti CLL-1 CART cell, and in particular scCART, of the disclosure that binds to the CLL-1-expressing cell in combination with an effective amount of another therapy.

In one aspect, CLL-1 is considered to be a “cancer stem cell” marker in AML. Therefore, an anti-CLL1 CART cell, and in particular scCART, of the disclosure can prevent relapse of AML, or even treat AML that is mostly CLL-1-negative but with a “stem” population of CLL1+ cells (a CLL1-expressing cells).

In one aspect, the disclosure provides compositions and methods for treating subjects that have undergone treatment for a disease or disorder associated with elevated expression levels of CLL-1.

The treatment with the engineered immune cells according to the disclosure may be in combination with one or more therapies against cancer selected from the group of antibodies therapy, chemotherapy, cytokines therapy, dendritic cell therapy, gene therapy, hormone therapy, laser light therapy and radiation therapy.

Preferably, the treatment with the engineered immune cells according to the disclosure may be administered in combination (e.g., before, simultaneously or following) with one or more therapies against cancer selected from aracytine, cytosine arabinoside, amsacrine, daunorubicine, idarubicine, novantrone, mitoxantrone, vepeside, etoposide (VP16), arsenic trioxyde, transretinoic acid, combination of arsenic trioxyde, transretinoic acid, mechlorethamine, procarbazine, chlorambucil, and combination thereof.

According to a preferred embodiment of the disclosure, said treatment can be administrated into patients undergoing an immunosuppressive treatment. Indeed, the present disclosure preferably relies on cells or population of cells, which have been made resistant to at least one immunosuppressive agent due to the inactivation of a gene encoding a receptor for such immunosuppressive agent. In this aspect, the immunosuppressive treatment should help the selection and expansion of the T-cells according to the disclosure within the patient.

The administration of the cells or population of cells according to the present disclosure may be carried out in any convenient manner, including by aerosol inhalation, injection, ingestion, transfusion, implantation or transplantation. The compositions described herein may be administered to a patient subcutaneously, intradermaly, intratumorally, intranodally, intramedullary, intramuscularly, by intravenous or intralymphatic injection, or intraperitoneally. In one embodiment, the cell compositions of the present disclosure are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. The cells or population of cells can be administrated in one or more doses. In another embodiment, said effective amount of cells are administrated as a single dose. In another embodiment, said effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, said effective amount of cells or composition comprising those cells are administrated parenterally. Said administration can be an intravenous administration. Said administration can be directly done by injection within a tumor.

In certain embodiments of the present disclosure, cells are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizimab treatment for MS patients or efaliztimab treatment for psoriasis patients or other treatments for PML patients. In further embodiments, the T cells of the disclosure may be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycoplienolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Henderson, Naya et al. 1991; Liu, Albers et al. 1992; Bierer, Hollander et al. 1993).

In a further embodiment, the cell compositions of the present disclosure are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH.

In another embodiment, the cell compositions of the present disclosure are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present disclosure. In an additional embodiment, expanded cells are administered before or following surgery.

In certain embodiments of the present disclosure, anti-CLL-1 scCAR expressing cells are administered to a patient in conjunction (e.g., before, simultaneously or following) with a drug selected from aracytine, cytosine arabinoside, amsacrine, daunorubicine, idarubicine, novantrone, mitoxantrone, vepeside, etoposide (VP16), arsenic trioxyde, transretinoic acid, combination of arsenic trioxyde, transretinoic acid, mechlorethamine, procarbazine, chlorambucil, and combination thereof. In these embodiments anti-CLL-1 scCAR expressing cells may be resistant to the particular drug or combination of drugs that is (are) administered in conjunction with anti-CLL-1 scCAR expressing cells.

In other embodiments of the present disclosure, anti-CLL-1 scCAR expressing cells are administered to a patient in conjunction with a drug selected from cytarabine, anthracyclines, 6-thioguanine, hydroxyurea, prednisone, and combination thereof.

Acute myeloid leukemia (AML) that is characterized by the overproduction of immature myeloid cells in the bone marrow is the most common acute leukemia in adults and the second most common leukemia in children, resulting in an aggressive and heterogeneous cancer affecting normal hematopoetic functions. In recent decades, improvements in two AML standard treatments with chemotherapy and allogeneic hematopoietic stem cell transplantation (alloHSCT) are limited as the prognosis of refractory/relapsed AML remains poor and the 5-year survival rate is below 50%. Due to chemoresistance or severe and long-term toxic effects on healthy/non-cancerous tissues/organs, most patients eventually succumb to relapsed and/or progressive disease, suggesting novel therapeutic strategies including target therapy and immunotherapy are urgently needed.

Recent advances in immunotherapy provide an impressive breakthrough in the treatment of hematological malignancies. A special type of engineered autologous T cell combining the specificity of antibody-targeting recognition with the potent effector mechanisms of T cells is called the Chimeric Antigen Receptor (CAR) T cell. The therapeutic benefits of CAR-T cells had been demonstrated and approved by U.S. FDA with anti-CD19 CAR T cells which exhibited strong and long-lasting anti-tumor activity in acute lymphocytic leukemia (ALL). CAR consists of several essential parts, an extracellular antigen-binding domain derived from the single-chain variable fragment (scFv) of the targeting antibody, a transmembrane domain, one or more costimulation domains such as 4-1BB (CD137), CD28, or ICOS (CD278) and an intracellular signaling domain of the CD3-C. Target recognition by the scFv domain confers CAR-T cells in mediating the tumor cytotoxicity in a major histocompatibility complex (MHC)-independent manner, and avoids the immune escape by reduction of antigen processing and presentation during tumorigenesis. With MHC-independent antigen recognition, CAR T cells have many advantages including acting more specific than TCR, programmable to recognize any tumor antigens, manageable cytotoxicity capacity, higher proliferation and longer persistence, making CAR-T cell therapy a superior therapeutic choice in cancer treatment.

### Non-Limiting Embodiments

The disclosure is illustrated herein by the following embodiments, which should not be construed as limiting. Those skilled in the art will understand that this disclosure may be embodied in many different forms and should not be construed as limited to the embodiments set forth herein.
1. A chimeric antigen receptor (CAR) for Human C-type lectin-like molecule-1 (CLL-1) comprising a polypeptide comprising:
an extracellular antigen binding domain comprising a single heavy chain variable domain (VH) and a single light chain variable domain (VL);
a transmembrane domain; and
an intracellular signaling domain, wherein the single heavy chain variable domain comprises a CDR1, a CDR2 and a CDR3 as set forth in a first amino acid sequence selected from the group consisting of SEQ ID NO: 10, SEQ ID NO: 12, SEQ ID NO: 14, SEQ ID NO: 16, and SEQ ID NO: 18, and
wherein the single light chain variable domain comprises a CDR1, a CDR2, and a CDR3 as set forth in a second amino acid sequence selected from the group consisting of SEQ ID NO: 11, SEQ ID NO: 13, SEQ ID NO: 15, SEQ ID NO: 17, and SEQ ID NO: 19.
2. The CAR of embodiment 1, wherein the first amino acid sequence is of SEQ ID NO: 14.
3. The CAR of embodiment 1, wherein the second amino acid sequence is of SEQ ID NO: 15.
4. The CAR of embodiment 1-3, wherein the single heavy chain variable domain is located at a N-terminus side of the single light chain variable domain.
5. The CAR of embodiment 1-3, wherein the single heavy chain variable domain is located at a C-terminus side of the single light chain variable domain.
6. The CAR of embodiment 1-5, wherein the single heavy chain variable domain and single light chain variable domain are directly fused to each other via a peptide bond.
7. The CAR of embodiment 1-5, wherein the single heavy chain variable domain and the single light chain variable domain are linked to each other via a peptide linker.
8. The CAR of embodiment 7, wherein the peptide linker comprises no more than 50 amino acid residues.
9. The CAR of embodiments 1-8, wherein the transmembrane domain is derived from CD8 or CD28.
10. The CAR of embodiments 1-9, wherein the intracellular signaling domain comprises a primary intracellular signaling domain of an immune effector cell.
11. The CAR of embodiment 10, wherein the primary intracellular signaling domain is derived from CD3ζ.
12. The CAR of embodiments 1-11, wherein the intracellular signaling domain comprises a co-stimulatory signaling domain.
13. The CAR of embodiment 12, wherein the co-stimulatory signaling domain is derived from a molecule selected from the group consisting of CD28, 4-1BB, OX40, ICOS and combinations thereof.
14. The CAR of embodiment 12, wherein the co-stimulatory signaling domain is derived from CD28, 4-1BB, or a combination thereof.
15. The CAR of embodiment 12, wherein the co-stimulatory signaling domain comprises a cytoplasmic domain of CD28.
16. The CAR of embodiments 1-15, further comprising a hinge domain.
17. The CAR of embodiment 16, wherein the hinge domain is located between a C-terminus of the extracellular antigen binding domain and a N-terminus of the transmembrane domain.
18. The CAR of embodiments 16-17, wherein the hinge domain is derived from molecule selected from the group consisting of CD28, 4-1BB, OX40, ICOS and combinations thereof.
19. The CAR of embodiments 16-17, wherein the hinge domain is derived from CD28.
20. The CAR of embodiment 1-19, further comprising a signal peptide located at a N-terminus of the polypeptide.
21. The CAR of embodiment 20, wherein the signal peptide is derived from CD28.
22. A chimeric antigen receptor (CAR) for Human C-type lectin-like molecule-1 (CLL-1) comprising a polypeptide comprising:
an extracellular antigen binding domain comprising a single heavy chain variable domain (VH) and a single light chain variable domain (VL);
a transmembrane domain; and
an intracellular signaling domain,
   wherein the single heavy chain variable domain comprises a CDR1, a CDR2 and a CDR3 as set forth in an amino acid sequence of SEQ ID NO: 14, and
   wherein the single light chain variable domain comprises a CDR1, a CDR2, and a CDR3 as set forth in an amino acid sequence of SEQ ID NO: 15.
23. The CAR of embodiment 22, wherein the single heavy chain variable domain is located at a N-terminus of the single light chain variable domain.
24. The CAR of embodiment 22, wherein the single heavy chain variable domain is located at a C-terminus of the single light chain variable domain.
25. The CAR of embodiment 22-24, wherein single heavy chain variable domain and the single light chain variable domain are directly fused to each other via a peptide bond.
26. The CAR of embodiment 22-24, wherein the single heavy chain variable domain and the single light chain variable domain are linked to each other via a peptide linker.
27. The CAR of embodiment 26, wherein the peptide linker comprises no more than 50 amino acid residues.
28. The CAR of embodiments 22-27, wherein the transmembrane domain is derived from CD8 or CD28.
29. The CAR of embodiments 22-28, wherein the intracellular signaling domain comprises a primary intracellular signaling domain of an immune effector cell.
30. The CAR of embodiment 29, wherein the primary intracellular signaling domain is derived from CD3ζ.
31. The CAR of embodiments 22-30, wherein the intracellular signaling domain comprises a co-stimulatory signaling domain.
32. The CAR of embodiment 31, wherein the co-stimulatory signaling domain is derived from a molecule selected from the group consisting of CD28, 4-1BB, OX40, ICOS and combinations thereof.
33. The CAR of embodiment 31, wherein the co-stimulatory signaling domain is derived from CD28, 4-1BB, or a combination thereof.
34. The CAR of embodiment 31, wherein the co-stimulatory signaling domain comprises a cytoplasmic domain of CD28.
35. The CAR of embodiments 22-34, further comprising a hinge domain.
36. The CAR of embodiment 35, wherein the hinge domain is located between a C-terminus of the extracellular antigen binding domain and a N-terminus of the transmembrane domain.
37. The CAR of embodiments 35-36, wherein the hinge domain is derived from molecule selected from the group consisting of CD28, 4-1BB, OX40, ICOS and combinations thereof.
38. The CAR of embodiments 35-36, wherein the hinge domain is derived from CD28 or 4-1BB.
39. The CAR of embodiment 22-38, further comprising a signal peptide located at a N-terminus of the polypeptide.
40. The CAR of embodiment 39, wherein the signal peptide is derived from CD28 or 4-1BB.
41. A chimeric antigen receptor (CAR) for Human C-type lectin-like molecule-1 (CLL-1) comprising a polypeptide comprising:
an extracellular antigen binding domain comprising an anti-CLL-1 single heavy chain variable domain (VH) and anti-CLL-1 single light chain variable domain (VL);
a transmembrane domain derived from CD8, CD28, 4-1BB, or a combination thereof; and
an intracellular signaling domain is derived from CD8, CD28, 4-1BB, OX40, ICOS, or a combination thereof.
42. The CAR of embodiment 41, wherein the anti-CLL-1 single heavy chain variable domain comprises a CDR1, a CDR2 and a CDR3 as set forth in a first amino acid sequence selected from the group consisting of SEQ ID NO: 10, SEQ ID NO: 12, SEQ ID NO: 14, SEQ ID NO: 16, and SEQ ID NO: 18.
43. The CAR of embodiment 41, wherein the anti-CLL-1 single light chain variable domain comprises a CDR1, a CDR2, and a CDR3 as set forth in a second amino acid sequence selected from the group consisting of SEQ ID NO: 11, SEQ ID NO: 13, SEQ ID NO: 15, SEQ ID NO: 17, and SEQ ID NO: 19.
44. The CAR of embodiment 41, wherein the anti-CLL-1 single heavy chain variable domain comprises a CDR1, a CDR2 and a CDR3 as set forth in the VH domain comprising an amino acid sequence of SEQ ID NO: 14.
45. The CAR of embodiment 41, wherein the anti-CLL-1 single light chain variable domain comprises a CDR1, a CDR2, and a CDR3 as set forth in the VL domain comprising an amino acid sequence of SEQ ID NO: 15.
46. The CAR of embodiment 41-45, wherein the anti-CLL-1 single heavy chain variable domain is located at a N-terminus of the anti-CLL-1 single light chain variable domain.
47. The CAR of embodiment 41-45, wherein the anti-CLL-1 single heavy chain variable domain is located at a C-terminus of the anti-CLL-1 single light chain variable domain.
48. The CAR of embodiment 41-47, wherein the anti-CLL-1 single heavy chain variable domain and the anti-CLL-1 single light chain variable domain are directly fused to each other via a peptide bond.
49. The CAR of embodiment 41-47, wherein the anti-CLL-1 single heavy chain variable domain and the anti-CLL-1 single light chain variable domain are linked to each other via a peptide linker
50. The CAR of embodiment 49, wherein the peptide linker comprises no more than 50 amino acid residues.
51. The CAR of embodiments 41-50, wherein the transmembrane domain is derived from CD8 or CD28.
52. The CAR of embodiments 41-51, wherein the intracellular signaling domain comprises a primary intracellular signaling domain of an immune effector cell.
53. The CAR of embodiment 52, wherein the primary intracellular signaling domain is derived from CD3ζ.
54. The CAR of embodiments 41-53, wherein the intracellular signaling domain comprises a co-stimulatory signaling domain.
55. The CAR of embodiment 54, wherein the co-stimulatory signaling domain is derived from CD28, 4-1BB, or a combination thereof.
56. The CAR of embodiment 54, wherein the co-stimulatory signaling domain comprises a cytoplasmic domain of CD28.
57. The CAR of embodiments 41-56, further comprising a hinge domain.
58. The CAR of embodiment 57, wherein the hinge domain is located between a C-terminus of the extracellular antigen binding domain and a N-terminus of the transmembrane domain.
59. The CAR of embodiment 57-58, wherein the hinge domain is derived from molecule selected from the group consisting of CD28, 4-1BB, OX40, ICOS, and combinations thereof.
60. The CAR of embodiment 57-58, wherein the hinge domain is derived from CD28.
61. The CAR of embodiment 41-60, further comprising a signal peptide located at a N-terminus of the polypeptide.
62. The CAR of embodiment 61, wherein the signal peptide is derived from CD28.
63. A chimeric antigen receptor (CAR) for Human C-type lectin-like molecule-1 (CLL-1) comprising:
a polypeptide comprising an amino acid sequence selected from the group consisting of SEQ ID NOs: 20 to 26.
64. The CAR of embodiment 63, wherein the polypeptide comprises amino acid sequence is selected from the group consisting of SEQ ID NOs: 25 to 26.
65. A chimeric antigen receptor (CAR) for Human C-type lectin-like molecule-1 (CLL-1) comprising:
a polypeptide derived from nucleic acid sequence selected from the group consisting of SEQ ID NOs: 27 to 33.
66. The CAR of embodiment 65, wherein the polypeptide comprises nucleic acid sequence is selected from the group consisting of SEQ ID NOs: 32 to 33.
67. A chimeric antigen receptor (CAR) for Human C-type lectin-like molecule-1 (CLL-1) comprising a polypeptide comprising:
an extracellular antigen binding domain comprising a single heavy chain variable domain (VH) and a single light chain variable domain (VL);
a transmembrane domain; and
an intracellular signaling domain,
wherein the single heavy chain variable domain comprises a CDR1, a CDR2 and a CDR3 as set forth in a first amino acid sequence selected from the group consisting of SEQ ID NO: 10, SEQ ID NO: 12, SEQ ID NO: 14, SEQ ID NO: 16, and SEQ ID NO: 18, or wherein the single light chain variable domain comprises a CDR1, a CDR2, and a CDR3 as set forth in a second amino acid sequence selected from the group consisting of SEQ ID NO: 11, SEQ ID NO: 13, SEQ ID NO: 15, SEQ ID NO: 17, and SEQ ID NO: 19.
68. The CAR of embodiment 67, wherein the first amino acid sequence is of SEQ ID NO: 14.
69. The CAR of embodiment 67-68, wherein the second amino acid sequence is of SEQ ID NO: 15.
70. The CAR of embodiment 67-69, wherein the single heavy chain variable domain is located at a N-terminus of the single light chain variable domain.
71. The CAR of embodiment 67-69, wherein the single heavy chain variable domain is located at a C-terminus of the single light chain variable domain.
72. The CAR of embodiments 67-71, wherein the single heavy chain variable domain and the single light chain variable domain are directly fused to each other via a peptide bond.
73. The CAR of embodiment 67-71, wherein the single heavy chain variable domain and the single light chain variable domain are linked to each other via a peptide linker.
74. The CAR of embodiment 73, wherein the peptide linker comprises no more than 50 amino acid residues.
75. The CAR of embodiments 67-74, wherein the transmembrane domain is derived from CD8 or CD28.
76. The CAR of embodiments 67-75, wherein the intracellular signaling domain comprises a primary intracellular signaling domain of an immune effector cell.
77. The CAR of embodiment 76, wherein the primary intracellular signaling domain is derived from CD3ζ.
78. The CAR of embodiments 67-77, wherein the intracellular signaling domain comprises a co-stimulatory signaling domain.
79. The CAR of embodiment 78, wherein the co-stimulatory signaling domain is derived from a molecule selected from the group consisting of CD28, 4-1BB, OX40, ICOS and combinations thereof.
80. The CAR of embodiment 78, wherein the co-stimulatory signaling domain is derived from CD28, 4-1BB, or a combination thereof.
81. The CAR of embodiment 78, wherein the co-stimulatory signaling domain comprises a cytoplasmic domain of CD28.
82. The CAR of embodiments 67-81, further comprising a hinge domain.
83. The CAR of embodiment 82, wherein the hinge domain is located between a C-terminus of the extracellular antigen binding domain and a N-terminus of the transmembrane domain.
84. The CAR of embodiments 82-83, wherein the hinge domain is derived from molecule selected from the group consisting of CD28, 4-1BB, OX40, ICOS and combinations thereof.
85. The CAR of embodiments 82-83, wherein the hinge domain is derived from CD28.
86. The CAR of embodiments 67-85, further comprising a signal peptide located at a N-terminus of the polypeptide.
87. The CAR of embodiment 86, wherein the signal peptide is derived from CD28.
88. An immune effector cell comprising the CAR of embodiments 1-87.
89. The immune effector cell of embodiment 88, wherein the immune effector cell is a T cell.
90. A pharmaceutical composition comprising the immune effector cell of embodiment 88-89, and a pharmaceutically acceptable carrier.
91. A method of treating a cancer that expresses CLL-1 in an individual, comprising administering to the individual an effective amount of the immune effector cell of embodiment 88-89 or the pharmaceutical composition of embodiment 90.
92. The method of embodiment 91, wherein the cancer is multiple myeloma.
93. The method of embodiment 91, wherein the cancer is refractory or relapsed multiple myeloma.
94. The method of embodiment 91, wherein the cancer is myeloid leukemia.
95. The method of embodiment 91, wherein the cancer is refractory or relapsed myeloid leukemia.

### NON-LIMITING EXAMPLES

The disclosure is illustrated herein by the experiments described by the following examples, which should not be construed as limiting. Those skilled in the art will understand that this disclosure may be embodied in many different forms and should not be construed as limited to the examples set forth herein.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

The present disclosure, in some embodiments, relates to development of CAR-T cells against CLL-1 antigens expressed on AML blasts and leukemia stem cells. Through the process of CAR-T cell hit selection, lead optimization with swapping antibody variable regions and exchanging the co-stimulatory domain, 4-1BB or CD28, the disclosed CAR-T cell candidates, 61H08 HL_2828z and 61H08 LH_2828z was selected with high CAR %, normal CD4/CD8 ratio, high cell expansion fold, more memory-like phenotypes of T_{scm} and T_{cm} (>80%) and the better cytotoxicity to AML cells both in vitro and in vivo. In AML U937 xenograft animal model, CLL-1 CAR-T cell candidates, 61H08 HL_2828z and 61H08 LH_2828z, significantly revealed the superior anti-tumor activity at dose of 3×10⁶ CAR-T cells per mouse and substantially persisted in the peripheral blood of xenograft mice. Besides, the disclosed CLL-1 CAR-T candidates, 61H08 HL 2828z and LH_2828z, was tolerable as they exerted no or limited colony formation inhibition of CD34+ cells derived from BM or PB at E/T ratio of 1 or 4 with incubation for 6 hours or 24 hours. Moreover, an ex vivo study of the disclosed CLL-1 (61H08 HL_2828z) CAR-T cells prepared with T cells from r/rAML patients, S-008 and S015, could mediate the obvious cytotoxicity to their paired primary AML blasts.

### Example I—Screening and Characterization of Anti-CLL-1 Antibodies

Biopanning with Phage-Displayed Human scFv Libraries

Human antibody phage libraries (DSyn2, SLE1, SLE2, SLE3, SLE3-1, SLE3-2, SS1-Mix and SS2) were used for screening of the antibodies against CLL-1 antigen by CRO service. Eight antibody libraries, including the fully synthetic human antibody library with diversity of 1×10¹⁰ (DSyn2) and seven human autoimmune disease-derived antibody libraries with diversity of 1×10⁹˜1×10¹⁰, were executed for bio-panning. Total 67 scFv antibodies with unique CDR sequences was obtained and then subjected to scFv-Fc format protein expression by recombinant DNA technology amplifying each unique DNA sequence into a mammalian expression vector. Of those, 58 scFv-Fc clones were successfully expressed and then assayed for cell-based CLL-1 antigen-binding by flow cytometry, six exemplary clones shown in Table 1.

TABLE 1
Exemplary clones screened by phage display

| ID | CDRH1 | CDRH2 | CDRH3 | CDRL1 | CDRL2 | CDRL3 |
|---|---|---|---|---|---|---|
| 59A09 | GFTFTDY | MISPSGG | AHDFDD | RASQSIR | DASTLQS | QQYNNSPLT |
| | YIH | GEAYYA | FDY | YSLA | | |
| | | DSVKG | | | | |
| 61A07 | GFTFSNH | SISGAGG | DESSYA | RANQSIR | AASTLQS | QQNTSSPST |
| | WMH | GKSYADS | YDGDY | NYLN | | |
| | | VKG | | | | |
| 61B07 | GFTFSDY | VISPGGS | TYRIEDF | RASQDM | NASTLQS | QQYSTTPT |
| | WMH | NIYYADS | DY | SSNLN | | |
| | | VKG | | | | |
| 61H08 | GFTFDDY | GIWPSGG | LLALTY | RASQGIS | DASTLQS | QQSYSTPFT |
| | GMH | NKEYAD | DYFDY | SYLA | | |
| | | SVKG | | | | |
| 65D01 | GGSFSGY | EINHSGS | SPLQWL | SGSSSNIG | DNNERPS | GTWDNSPS |
| | YWS | TNYNPSL | DDAFDI | SNYVS | | TDWV |
| | | KS | | | | |
| 65E04 | GFTFSSYE | YISSSGST | DYGDH | QGDSLRS | GKNNRPS | NSRDSSGNR |
| | MN | IYYADSV | YY | YYAS | | V |
| | | KG | | | | |
| 72C10 | GFTFSVN | TISGSDG | DVDSEI | RASQSVT | AASNLQS | QQTNNSPAT |
| | AMH | HKYYAD | GDGYHE | NALN | | |
| | | SVKS | DI | | | |

Screening of the Advanced Anti-CLL-1 scFv-Fc Hits by Flow Cytometry

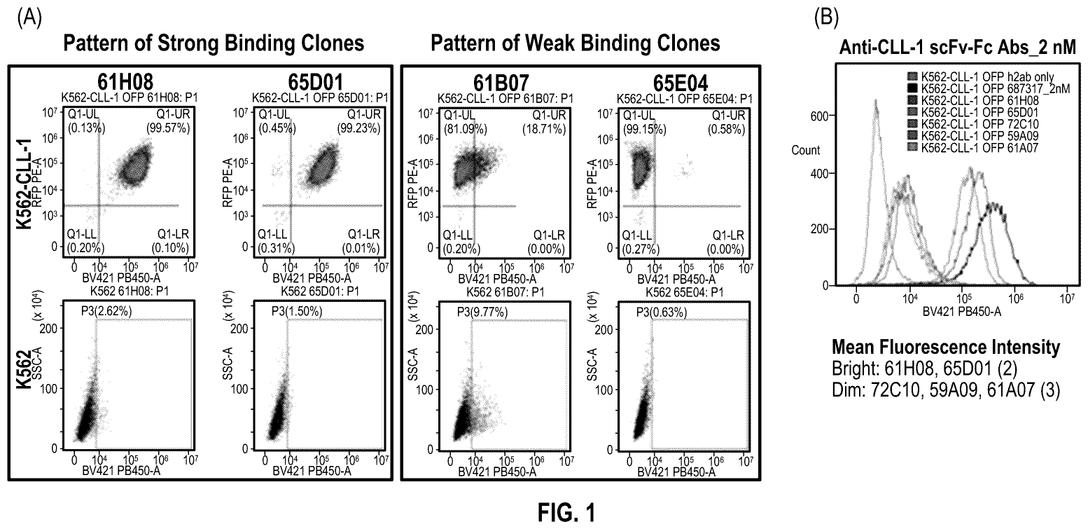

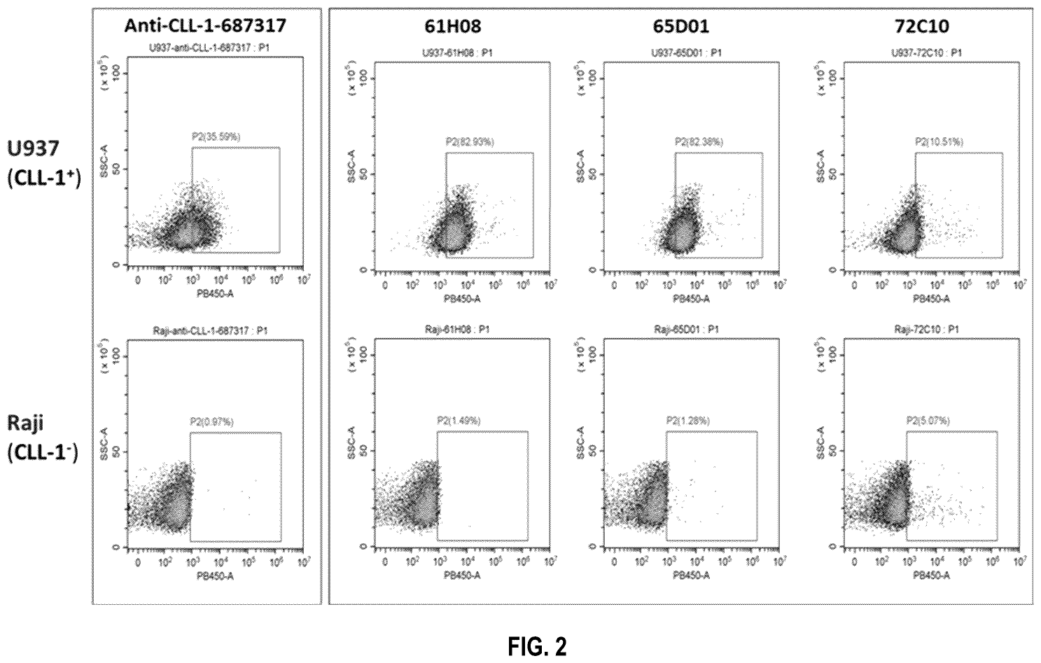

As illustrated in FIGS. 1A-B and 2, purified scFv-Fc antibodies were further assessed by flow cytometry for their binding to CLL-1 antigens of the K562-Luc-CLL-1 transfectant as well as the CLL-1+ AML cell line, U937, MOLM-14 and THP-1. K562 cells, a human erythroleukemic cell line, and Raji-Luc cells, a human B lymphoblastoid cell line, were used as the CLL-1-negative control cell lines. Each scFv-Fc antibody was screened with six cell lines, including K562, K562-CLL-1, U937, MOLM-14, THP-1 and Raji. Although the binding profiles of each clone varied with cell lines tested, the Top 3 clones with stronger binding pattern were selected, and the ranges of binding percentage were 52%˜99% for K562-Luc-CLL-1, 25%˜98% for U937, 6%˜37% for MLOM-14 and 1%-36% for THP-1.

Determination of the Interaction Between CLL-1 Antigens and the Advanced Anti-CLL-1 scFv-Fc Hits by AlphaLISA

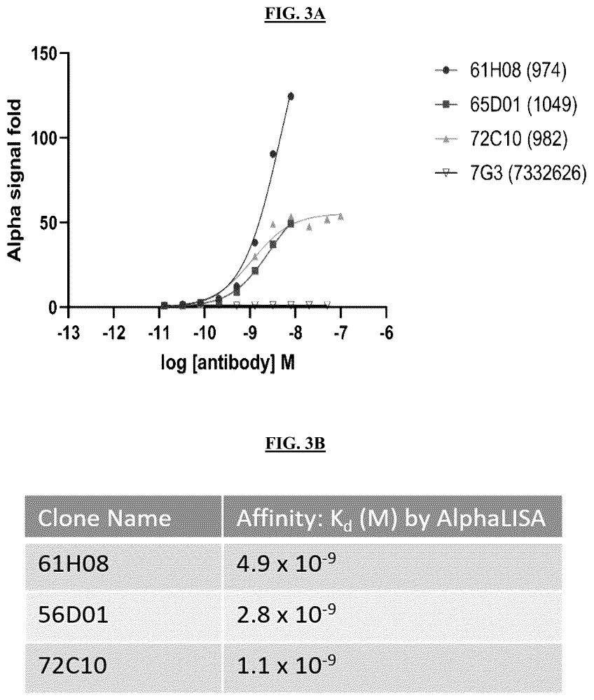

In order to characterize the antigen binding capacities of anti-CLL-1 scFv-Fc antibodies, an AlphaLISA sandwich assay was set up with Anti-6×His Acceptor beads, Protein A Donor beads, and CLL-1-ECD. By AlphaLISA assay, the K_{d} values of each anti-CLL-1 scFv-Fc antibody were determined. The range of K_{d} values was between 1.1×10⁻⁹ M and 4.9×10⁻⁹ M. The ranking order based on the maximum of AlphaLISA signal fold from high to low was 61H08, 56D01 and 72C10, as illustrated in FIG. 3.

### Example II—CAR Gene Design and Lentivirus Production

Design and Construction of CLL-1 CAR Genes with the Advanced Anti-CLL Antibody Hits

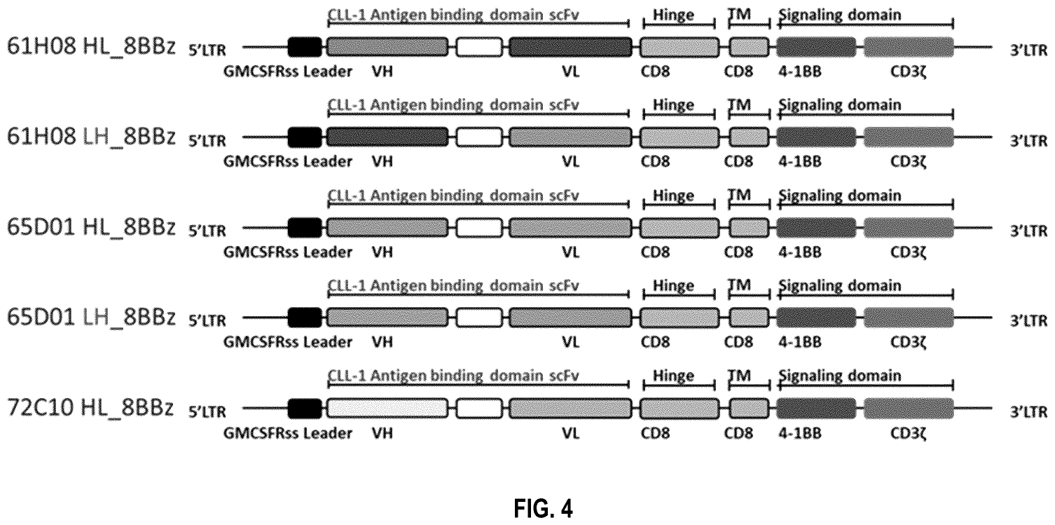

CAR Lentiviral plasmid is the key material for CAR-T cell generation and the 2nd generation CAR gene format including the essential elements in the order of the signal peptide GMCSFRss, scFv, the hinge and transmembrane regions of the CD8 molecule, the cytoplasmic portions of 4-1BB, the cytoplasmic component of the CD3ζ, T2A and enhanced GFP will be applied for CAR LV transfer plasmid construction, i.e., GMCSFRss-scFv-hinge-TM-costimulatory domain-CD3z-T2A-EGFP. The advanced anti-CLL antibody hits, 61H08, 65D01 and 72C10, were applied for CLL-1 CAR lentiviral vector construction, as illustrated in FIG. 4.

Each CLL-1 CAR gene was firstly generated in the pMK cloning vector by circular PCR method amplifying each anti-CLL-1 scFv fragment in frame with CD8 hinge/TM-4-1BB-CD34-T2A-enhanced GFP backbone in pMK cloning vector and then each promoterless CLL-1 CAR gene in cloning vector was sub-cloned into the lentiviral plasmid by In-Fusion technology.

### Productivity Assessment of CLL-1 CAR Lentiviral Vectors

Although CAR LV titers varied with CAR clones and its binding domain formats, i.e., scFv VH-VL or VL-VH orientation, all CLL-1 CAR LVs were successfully produced with high yields. These CLL-1 CAR LVs were then used for the transduction of MACSbead-isolated primary T cells from healthy donors' PBMCs, as shown in Table 2.

TABLE 2
Productivity assessment of CLL-1 CAR lentiviruses

| | | | Viral Titer: TU/ml |
|---|---|---|---|
| Clone | Construct | Production batch | (Supernatant) |
| 61H08 | HL1 | Batch 1 | 3.86E+06 |
| | | Batch 2 | 8.22E+06 |
| | LH1 | Batch 1 | 1.00E+07 |
| 65D01 | HL1 | Batch 1 | 3.21E+06 |
| | LH1 | Batch 1 | 1.42E+07 |
| 72C10 | HL1 | Batch 1 | 3.05E+06 |

### Optimization Design of CLL-1 (61H08) CAR Gene Formats by Swapping Antibody Variable Regions and Exchanging the Co-Stimulatory Domain

By in vitro functional assays, CLL-1 (61H08 HL_8BBz and 61H08 LH_8BBz) CAR-T cells were selected as the advanced hits, and their CAR gene formats were further optimized by scFv VH-VL swapping and co-stimulatory domain exchange in order to maximize their in vitro and in vivo CAR-T cell potency. In addition, two more CLL-1 CAR-T cells of 65D01 LH_8BBz and one reference, 24C8 HL_8BBz, and one control CD19 (FMC63) CAR-T cell were included for potency evaluation with CLL-1 CAR-T cell leads.

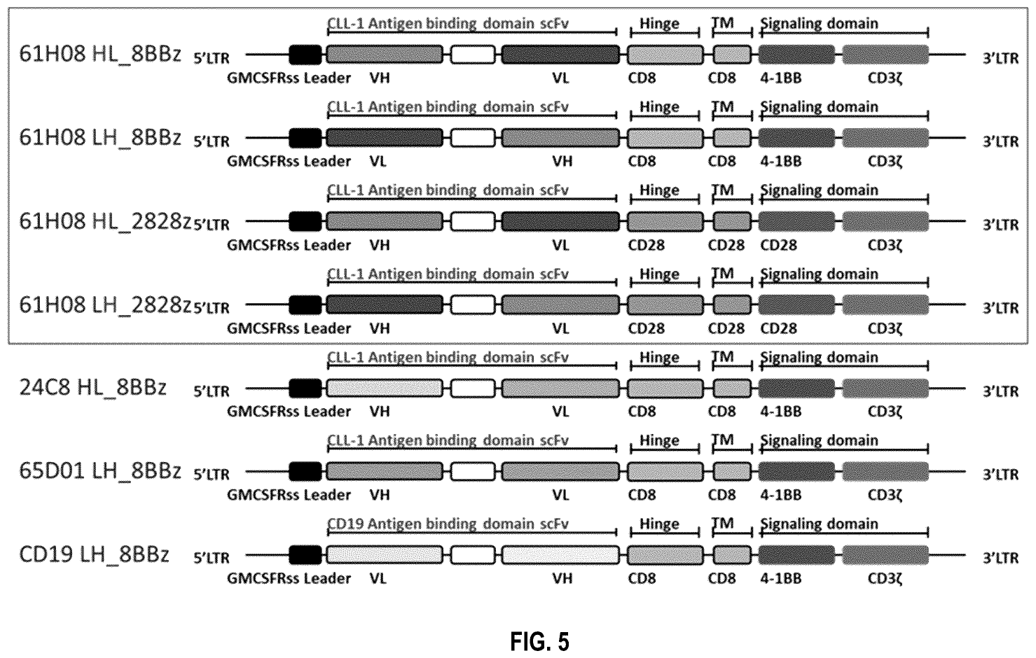

CLL-1 (61H08 HL_8BBz and 61H08 LH_8BBz) CAR genes were further optimized by co-stimulatory domain exchange with CD28 hinge, CD28 transmembrane domain and CD28 co-stimulatory domain, as illustrated in FIG. 5.

### Example III—Production of CLL-1 CAR-T Cell Clones

### CLL-1 CAR-T Cell Bioprocess

The peripheral blood mononuclear cells (PBMCs) of healthy donor were collected by centrifugation, and CD3+ T cells isolated with CD3 microbeads were then stored in liquid nitrogen tank before executing the CAR-T cell process. Thawed T cells were stimulated by anti-CD3/anti-CD28 Ab-coated Human T-Activator beads, and cultured. The activated T cells were transduced with a lentiviral vector encoding CD19 or CLL-1 CAR constructs. The transduction efficiency of each CAR T cell was analyzed by flow cytometry and the CAR-T cell culture was then subcultured and expanded. Afterwards, the CAR-T cells were harvested and analyzed for CAR % and phenotyping before cryopreserving in the liquid nitrogen vapor phase.

### Productivity Assessment of CLL-1 CAR-T Cell Clones

Different CLL-1 CAR-T cells (different CLL-1 clones) were manufactured to observe the bioprocessing attributes, such as CAR %. Two batches of production were executed with donor 25 and donor 26.

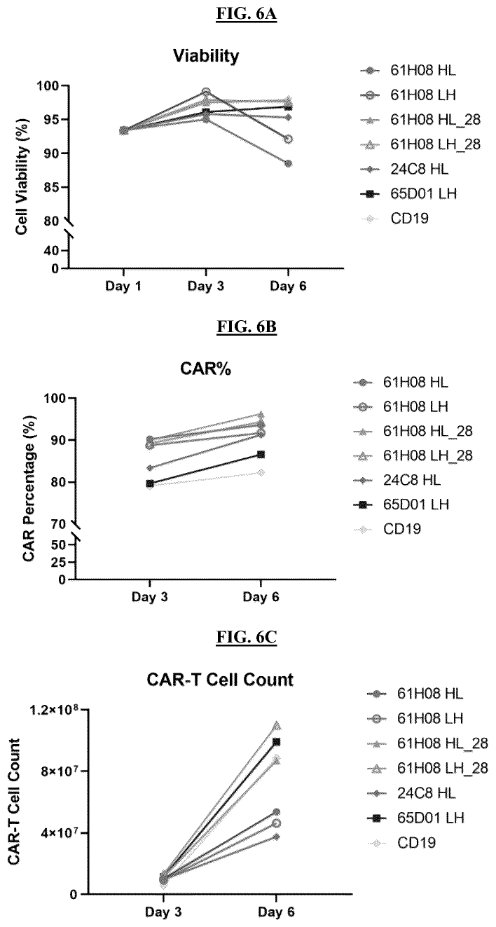

As illustrated in FIG. 6A-C, for CLL-1 CAR-T cells derived from donor 25, most CLL-1 CAR-T clones had above 95% viability throughout the culture period. 61H08 HL and 61H08 LH had a decrease in viability on day 6, but the viabilities were still around 90%. As for CAR %, 61H08 HL 28 and 61H08 LH_28 had the highest CAR % around 95%, while 65D01 LH had the lowest CAR % about 88%. Regarding to cell expansion, it was noted that 61H08 HL 28 and 61H08 LH_28 had significant growth advantage compared to their 4-1BB counterparts. In addition, 65D01 LH also had great growth advantage.

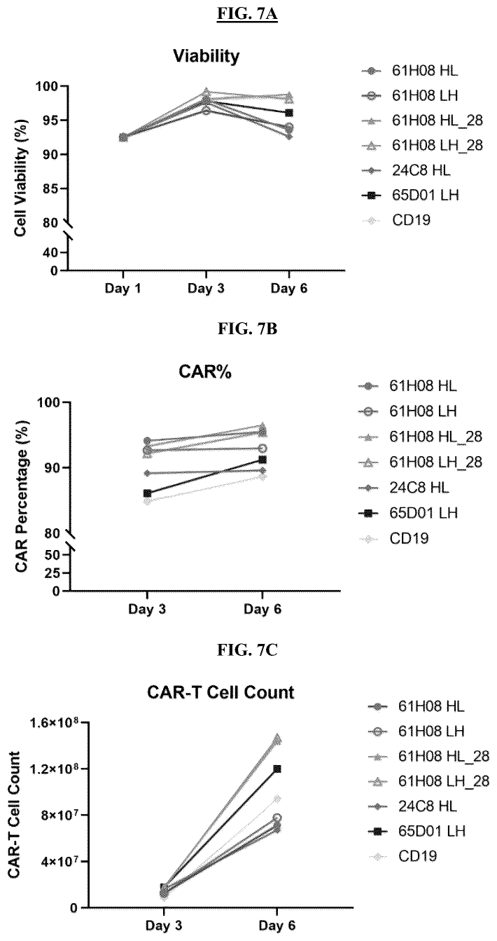

As illustrated in FIG. 7A-C, for CLL-1 CAR-T cells derived from donor 26, all CLL-1 CAR-T clones had above 90% viability throughout the culture period. As for CAR %, 61H08 HL 28 and 61H08 LH_28 had the highest CAR % around 95%, while 24C8 HL had the lowest CAR % about 90%. Regarding to cell expansion, 61H08 HL_28 and 61H08 LH_28 had significant growth advantage compared to their 4-1BB counterparts. In addition, 65D01 LH also had great growth advantage.

In a nutshell, two batches of CLL-1 CAR-T production had similar bioprocessing attributes. 61H08 HL_28 and 61H08 LH_28 had higher viability, CAR % and expansion fold compared to their 4-1BB counterparts. Moreover, of all the CLL-1 CAR-T clones, 61H08 HL_28 and 61H08 LH_28 had the best bioprocessing properties.

### Phenotypic Profiling of CLL-1 CAR-T Cell Clones

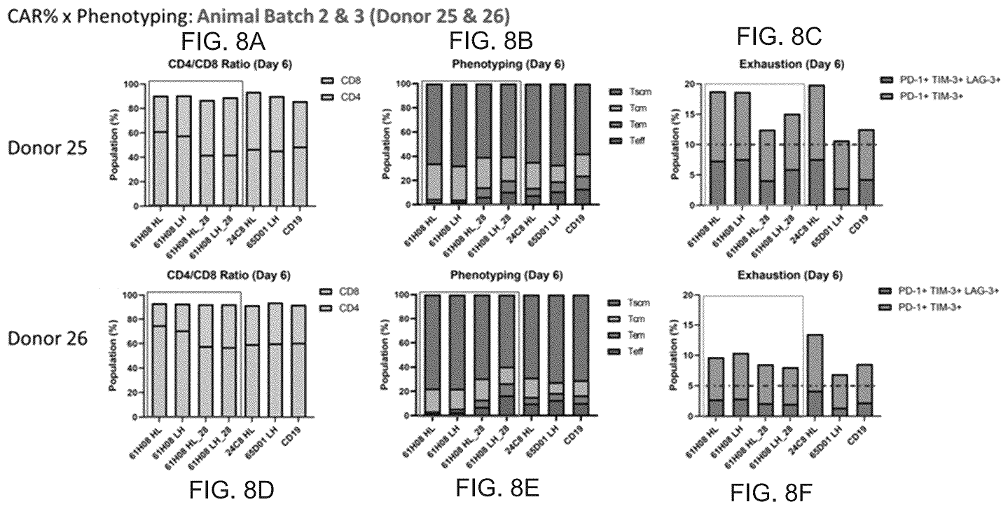

The phenotypes and CAR expression levels of CLL-1 CAR-T cell clones were monitored consecutively. Memory phenotypic profiles and the expression of inhibitory markers were analyzed. Functional binding of CAR or CAR expression was detected via antigen labelling method. Looking at CD4/CD8 ratio, 61H08 HL_28 and 61H08 LH_28 had more CD8 population compared to their 4-1BB counterparts. 61H08 HL and 61H08 LH had higher CD4 populations, while others had similar CD4/CD8 ratio. As for memory phenotyping, 61H08 HL_28 and 61H08 LH_28 had higher populations of T_{eff} and Tₑₘ cells compared to their 4-1BB counterparts. 61H08 HL_BB and 61H08 LH_BB had their majority of population as T_{scm} and T_{cm}, while other CLL-1 CAR-T cell clones had similar memory phenotypic profiles. In terms of inhibitory marker expressions, PD-1, TIM-3, LAG3 triple positive cells are more likely to be exhausted cells. All of our CLL-1 CAR-T cell clones had low percentage of triple positive cells, with less than 10% in batch of donor 25 and less than 5% in batch of donor 26. Overall, all of our CLL-1 CAR-T cell clones had their majority of population as memory phenotypes and higher CD4 percentage, while 61H08 HL 28 and 61H08 LH_28 had higher percentage of Tₑₘ and T_{eff} cells (T_{scm} and T_{cm} still as the main population) and higher CD8 populations. Phenotypic profiles of CLL-1 CAR-T cells from donor 25 and 26 are illustrated in FIG. 8A-F.

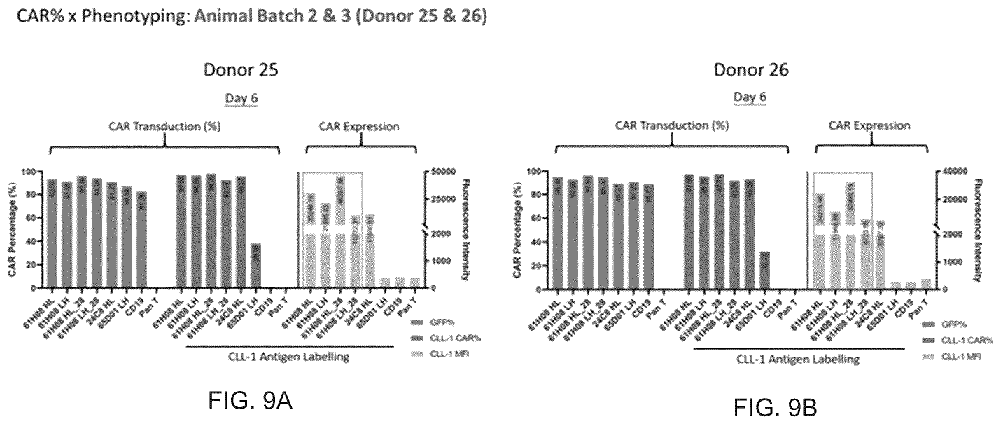

Using antigen labelling, we can detect the CAR expression and expression intensity of our CLL-1 CAR-T cell clones. The results showed that 65D01 LH_BB had low CAR expression and CAR expression intensity. The conversion of 61H08 HL_8BBz to 61H08 HL 2828z resulted in the upregulation of CAR expression, while the conversion of 61H08 LH 8BBz to 61H08 LH_2828z decreased the CAR expression. 61H08 HL_28 and 61H08 HL_BB had the highest CAR expression intensity followed by 61H08 LH_BB and 61H08 LH_28. All four disclosed CLL-1 CAR-T cell clones had better CAR expression than the reference 24C8 HL_BB. CAR expression profiles of CLL-1 CAR-T cells from donor 25 and 26 are illustrated in FIG. 9A-B.

### Example IV—Characterization of CLL-1 CAR-T Cell Clones

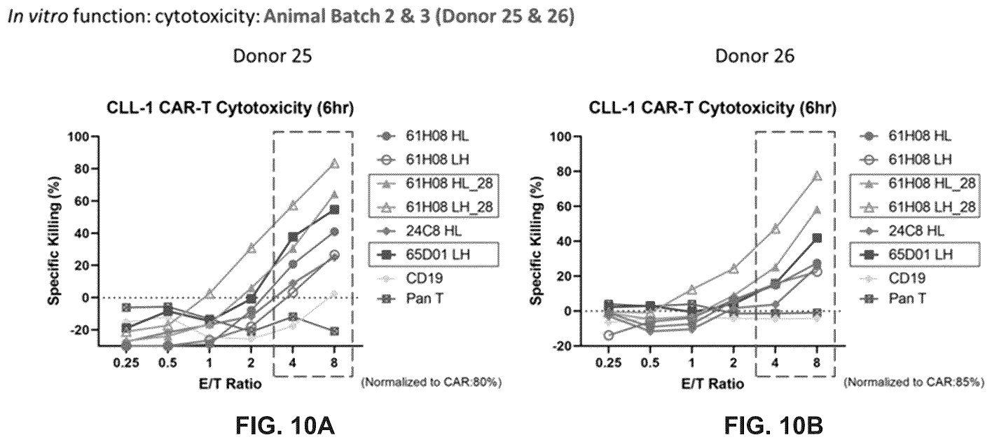

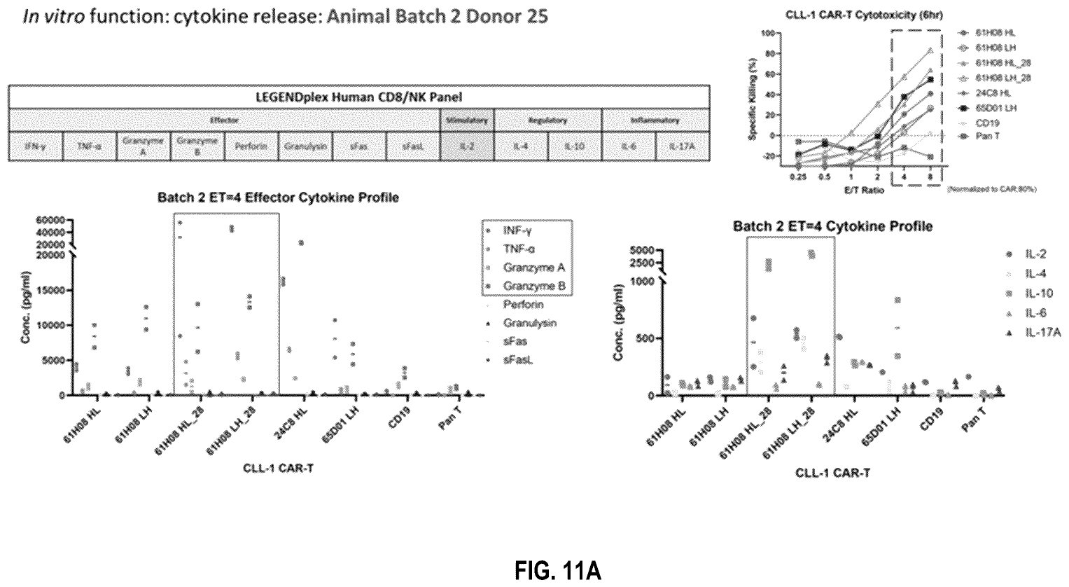

For in vitro cytotoxicity, it was noted that there were little to none non-specific killing by Pan T and CD19 CAR-T cells, indicating that all cytotoxicity observed was exerted by CLL-1 CAR-T cells. 61H08 HL_28 and 61H08 LH_28 had better cytotoxicity against CLL-1+U937 cells than their 4-1BB counterparts. 61H08 HL_28 and 61H08 LH_28 also were demonstrated to have the best cytotoxicity against the target cells as compared to all other CAR-T cell clones (24C8 HL_BB, 65D01 LH_BB). The cytotoxicity of CLL-1 CAR-T cell clones against CLL-1+U937 cells is illustrated in FIG. 10A-B.

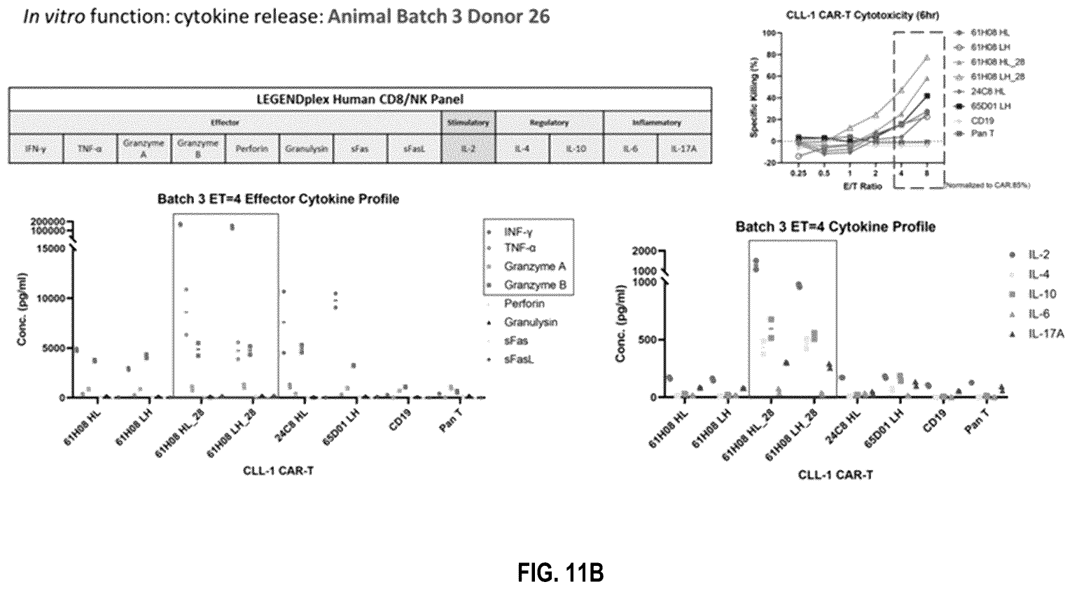

For in vitro cytokine release profiles, the cytokine profiles of CLL-1 CAR-T cell clones in cytotoxicity assay were analyzed via LegendPlex multiplex cytokine detection kit. The patterns of both batches were similar, with 61H08 HL_28 and 61H08 HL_28 having higher secretion of effector cytokines (IFN-γ, TNF-α, Granzyme B), stimulatory cytokine (IL-2), regulatory cytokines (IL-4, IL-10) and inflammatory cytokine (IL-17A). The cytokine releasing profiles correspond to the cytotoxicity outcomes showing that 61H08 HL_28 and 61H08 LH_28 having the best tumor-killing abilities compared to other CLL-1 CAR-T cell clones. The in vitro cytokine release profiles of CLL-1 CAR-T Cell clones encountered with CLL-1+U937 cells are illustrated in FIG. 11A-B.

### CLL-1 CAR-T Cell-Mediated In Vivo Anti-AML Tumor Activities in U937-Xenograft Model

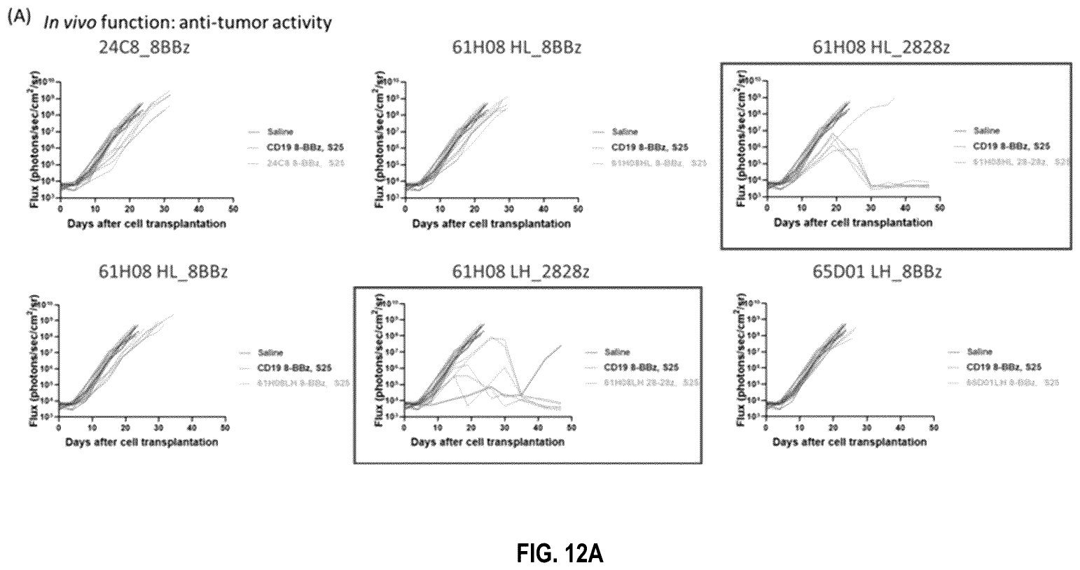

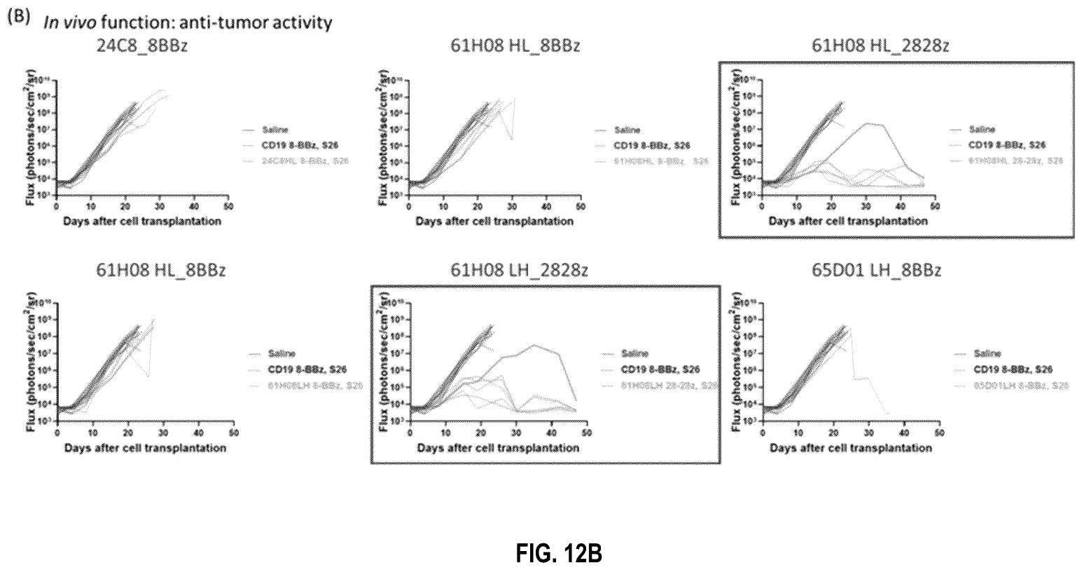

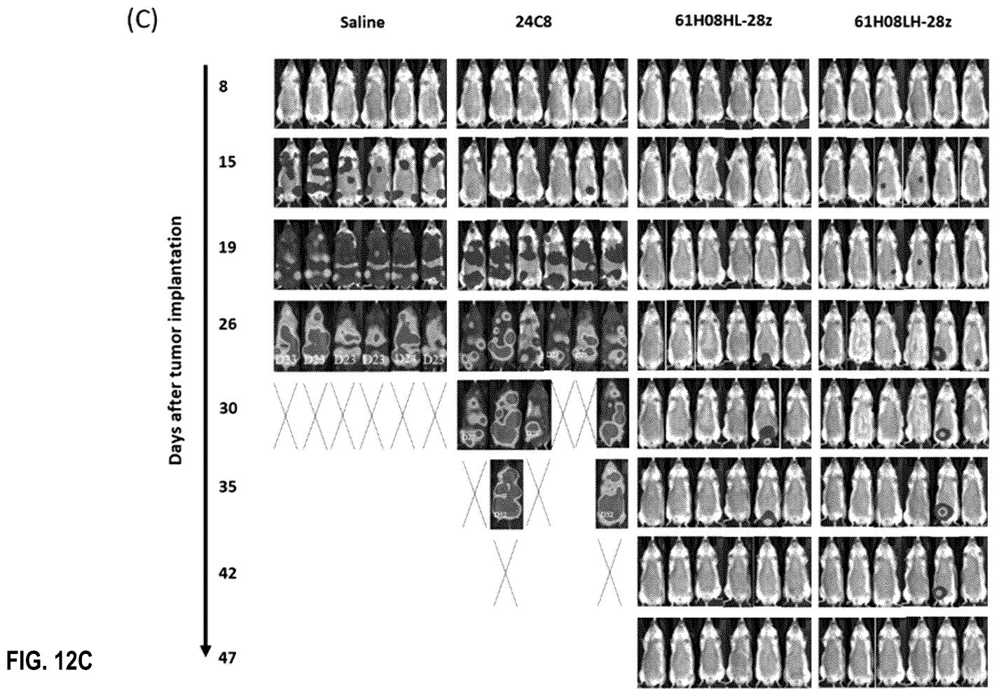

A xenogeneic U937-Luc AML model was established to test the anti-AML in vivo activity of CLL-1 CAR-T cells. The AML cell expansion was weekly measured using bioluminescence imaging (BLI). In the setting, mice receiving U937 transplant at 1×10⁴ per mouse were dead around 25 days post tumor inoculation. It was demonstrated that 61H08 (HL or LH) CAR-T cells with CD28 co-stimulatory domain exhibited better anti-AML activity than those with 4-1BB domain and other CLL-1 CAR-T cell clones, i.e., clone 24C8_8BBz and 65D01 LH 8BBz. Of note, 61H08 HL 2828z had a more uniform tumor inhibition effect shown in both donors. From BLI images, it was shown that 61H08 HL 2828z and 61H08 LH_2828z had significant tumor inhibition effects compared to 24C8 HL_8BBz reference clone and the control CD19 CAR-T cell clone. The BLI Data of CLL-1 CAR-T Cell clones in U937 Xenograft Model are illustrated in FIG. 12A-C.

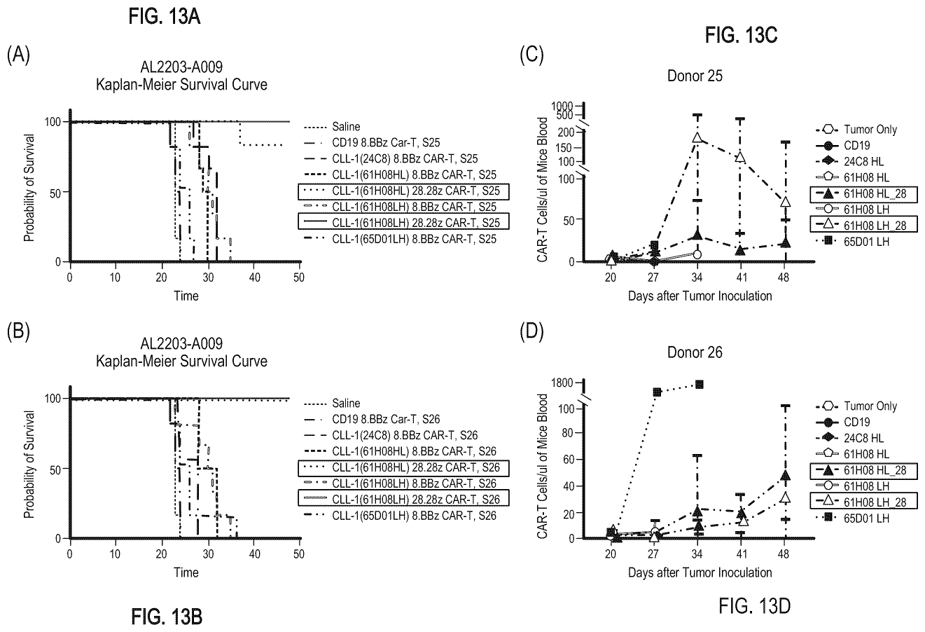

For xenograft mice survival, mice that received CLL-1 CAR T cells exhibited a modest improvement in survival compared to the groups treated with saline (PBS) or control CD19 CAR-T cells. In particular, CLL-1 (61H08 HL or LH) CAR-T cells with CD28 co-stimulatory domain prolonged the superior survival time of U937 xenograft mice compared to other CAR-T cell clones with only one mouse died in 61H08 HL_2828z group from donor 25. For in vivo CAR-T cell persistence, 65D01 LH_8BBz from donor 26 had one mice having extremely high amount of CAR-T cells in the blood of the mouse, which eventually died probably resulting from xGVHD. 61H08 HL 2828z and 61H08 LH_2828z had the best in vivo persistence compared to other CLL-1 CAR-T cell clones. There was no significant difference in the in vivo persistence between 61H08 HL 2828z and 61H08 LH_2828z. The Kaplan-Meier survival curves of xenograft mice receiving CAR-T cells and the in vivo CLL-1 CAR-T cell persistence are illustrated in FIG. 13A-D.

### In Vivo Cytokine Release Profiles

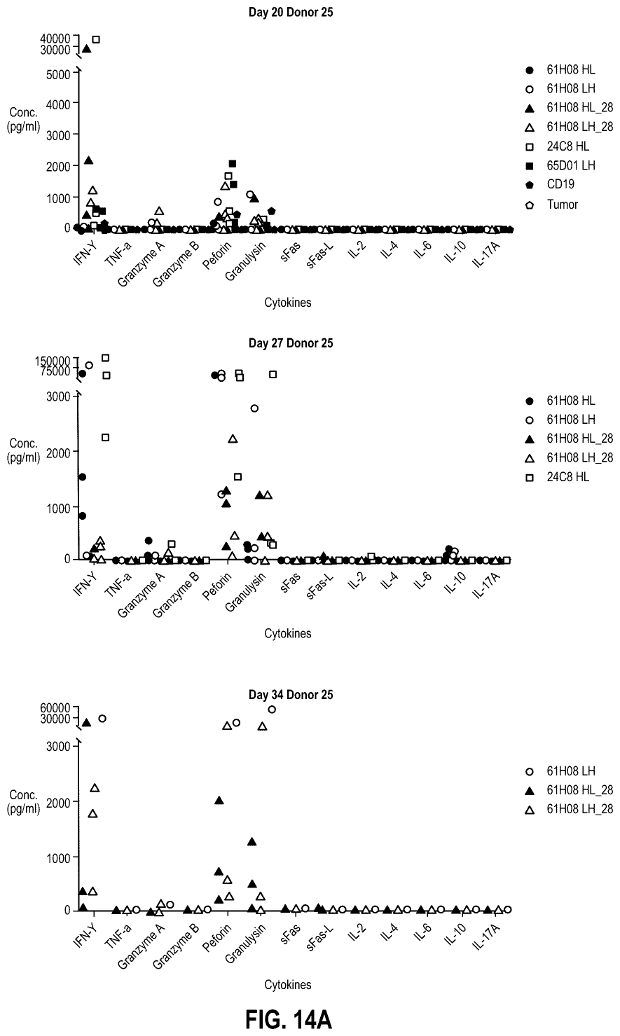

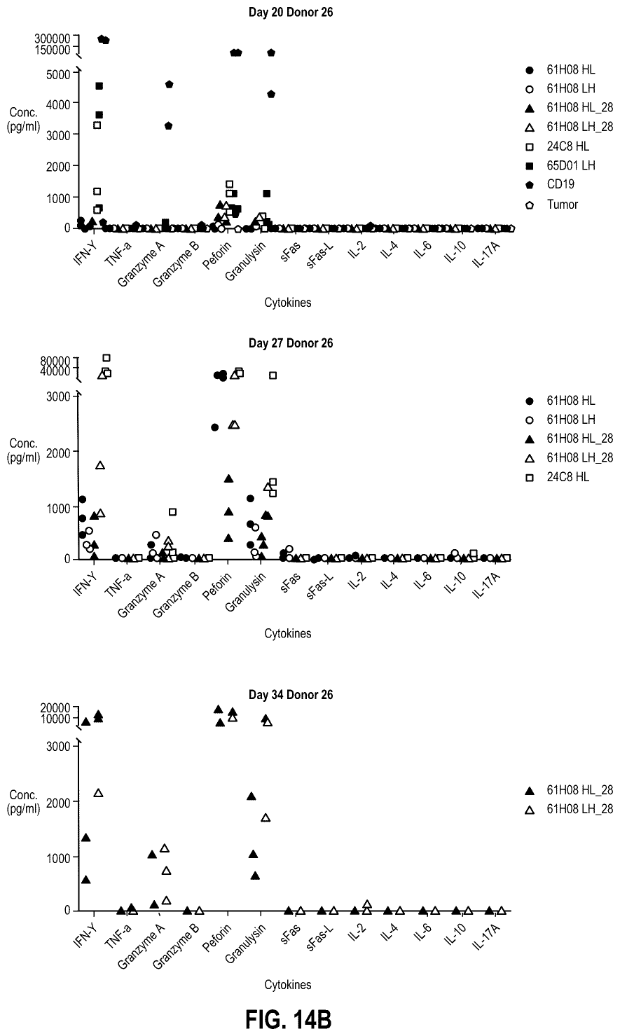

Plasma samples of U937-bearing mice post CAR-T cell infusion on day 20, 27 and 34 were assayed for cytokine release profiles. It was found that the responsive effector cytokines of CAR-T cells engaged with target cells, such as IFN-γ, granzyme A, perforin and granulysin, were detected in the plasma and their levels were increased along with time. Although these cytokine profiles varied with CLL-1 CAR-T cell clones in kinetics, CLL-1 (61H08 HL or LH) CAR-T cells with CD28 co-stimulatory domain in survived mice on Day 34 still released substantial amounts of these effector cytokines, implying the active process to eliminate the implanted AML tumor cells. In the aspect of releasing stimulatory, regulatory or inflammatory cytokines, IL-2 (stimulatory) and IL-10 (regulatory) were occasionally detected in the following plasma samples. In vivo cytokine release profiles of xenograft mice receiving CLL-1 CAR-T cell clones are illustrated in FIG. 14A-B.

### Assessment of Hematotoxicity by Stem Cell (CD34+) Colony Formation Assay

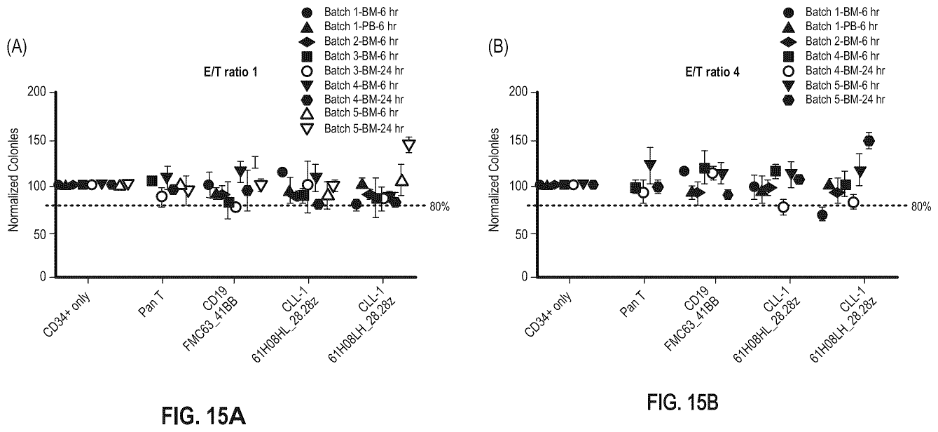

The hematotoxicity assessment of disclosed CLL-1 CAR-T cell clones were conducted in colony formation assay with CD34+ stem cells derived from normal bone marrow (BM) or peripheral blood (PB). It was noted that disclosed CLL-1 CAR-T cell candidates, 61H08 HL 2828z and 61H08 LH_2828z, exerted no or limited colony formation inhibition of CD34+ cells derived from BM or PB at E/T ratio of 1 or 4 with incubation for 6 hours or 24 hours. The hematotoxicity assessment of disclosed CLL-1 CAR-T cell candidates is illustrated in FIG. 15A-B.

### Production of CLL-1 CAR-T Cell Candidate (ARD103)

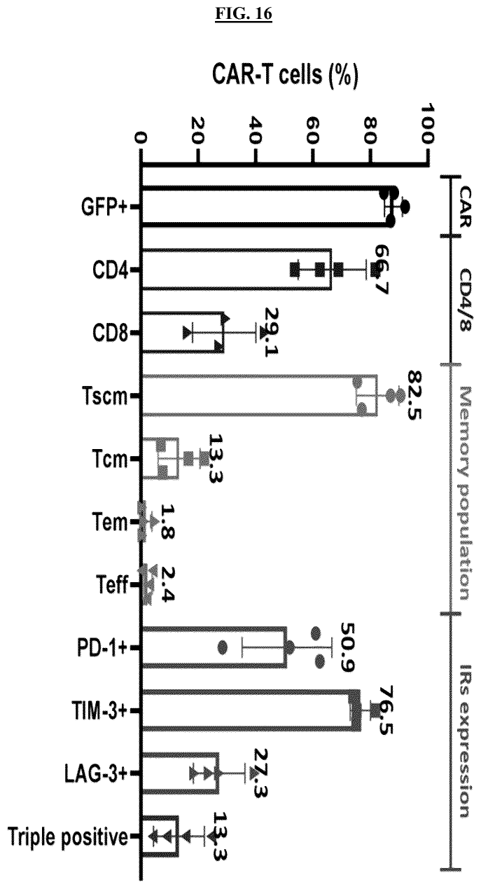

CLL-1 CAR-T cell candidate, 61H08 HL_2828z, was produced by a short bioprocess procedure. The CLL-1 CAR-T cells retained high cell expansion fold and had characteristics of high CAR % (>80%), regular CD4/CD8 ratio, high percentages of memory-like phenotypes of T_{scm} and T_{cm} (>70%) and relative low expression levels of triple positive inhibitory markers (PD-1+ TIM-3+ LAG-3+). Characteristics of the processed CLL-1 CAR-T cell candidate are illustrated in FIG. 16.

### CLL-1 CAR-T Cell Candidate Mediated In Vitro Target-Specific Immune Responses

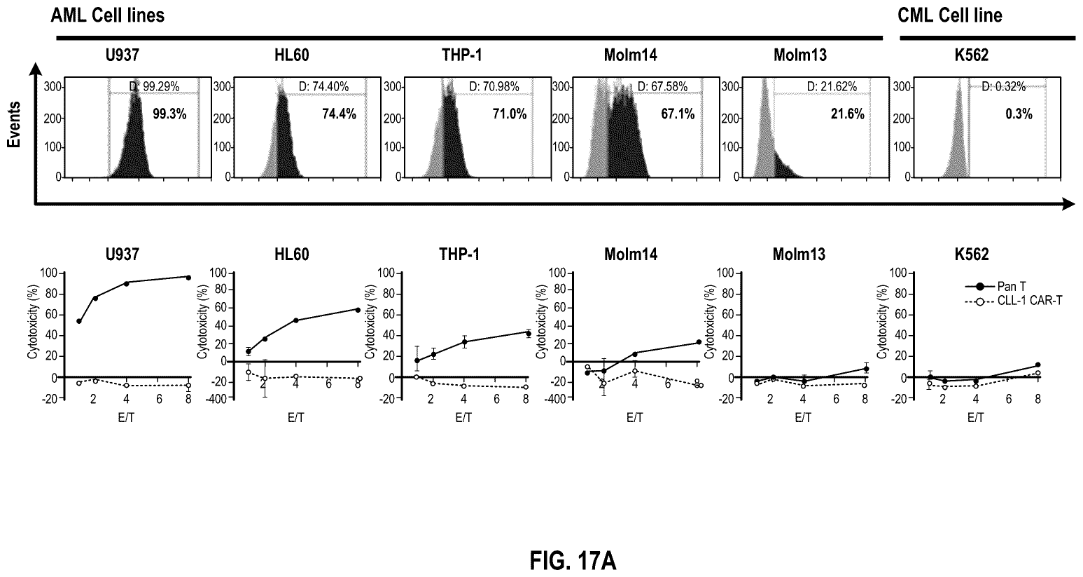

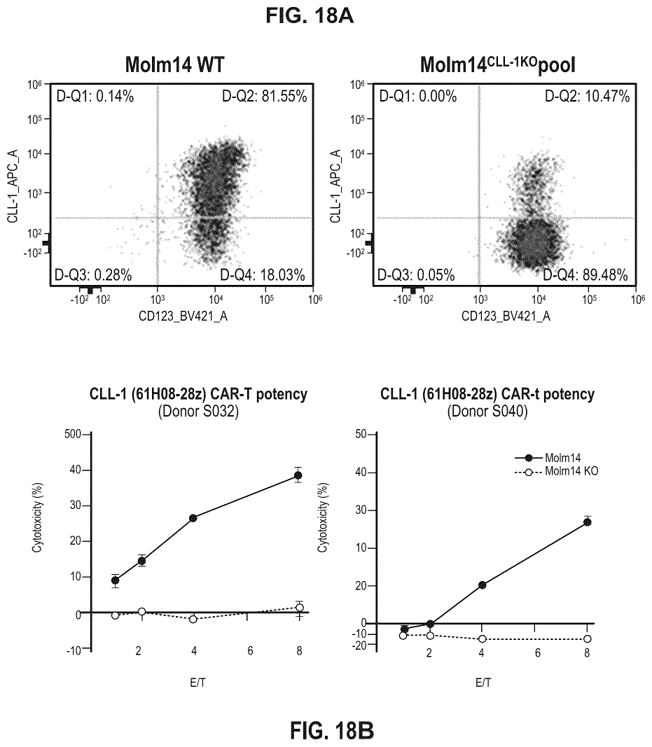

CLL-1 CAR-T cell candidate-mediated cytotoxicity is CLL-1 antigen-specific and correlated with CLL-1 antigen expression levels. The luciferase-based reporter AML cell lines were generated by transduction with lentiviral vectors encoding the luciferase reporter gene and were assessed with the comparable expression levels of CLL-1 antigens to their parental cells. Of note, AML cell lines expressed higher CLL-1 antigens, such as U937, THP-1, HL-60 and MOLM-14, are susceptible to ARD103-mediated cytotoxicity than those expressed lower or no CLL-1 antigens, such as MOLM-13 and K562 CML cell line, respectively. Knockout of CLL-1 gene by CRISPR-Cas9 in MOLM-14 cell pools render them not responding to ARD103-mediated cytotoxicity, further confirming that ARD103 is specific to CLL-1 antigens. CLL-1 CAR-T cell candidate-mediated cytotoxicity is correlated with CLL-1 antigen expression levels, as illustrated in FIG. 17A-C. CLL-1 CAR-T cell candidate-mediated cell killing is CLL-1 antigen specific, as illustrated in FIG. 18A-B.

### CLL-1 CAR-T Cell Candidate Exhibited Anti-Tumor Activities at Minimal Effective Dose

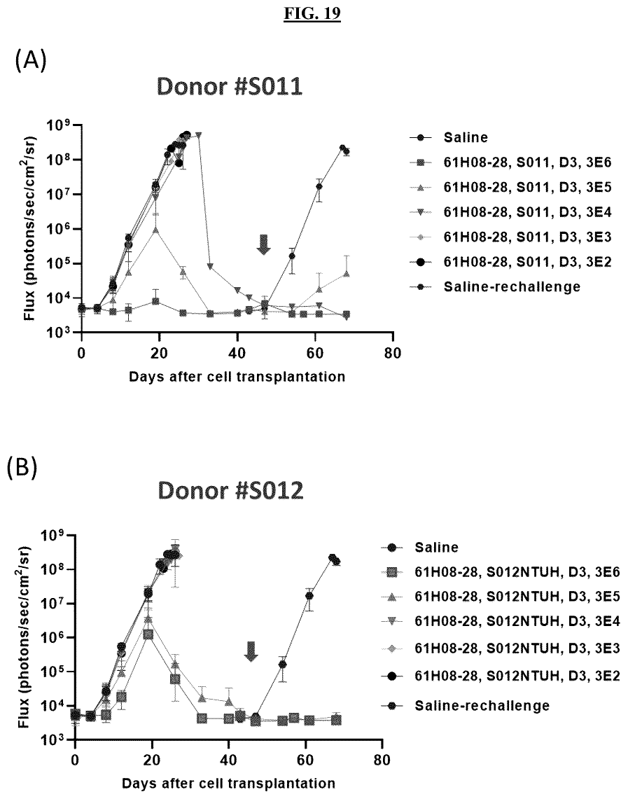

In xenogeneic U937-Luc AML model, mice were infused with descending doses of CLL-1 CAR cell candidate, 61H08 HL_2828z, prepared from two donors by a short bioprocess procedure. 61H08 HL_2828z by fast production from either donor exerted a dose-dependent tumor growth inhibition effect and the minimal effective dose was titrated at as low as dose of 3×10⁵ per mouse. Treatment of xenograft mice with 61H08 HL_2828z prolonged mice survival and CAR-T cells persisting in the peripheral blood elicited effective recall responses after tumor cell re-challenge. The MED and persistent anti-tumor activities of CLL-1 CAR-T cells are illustrated in FIG. 19A-B.

Ex Vivo Study of r/rAML Patient-Derived CLL-1 (61H08 HL_2828z) CAR-T Cells-Mediated Cytotoxicity to Primary AML Blasts

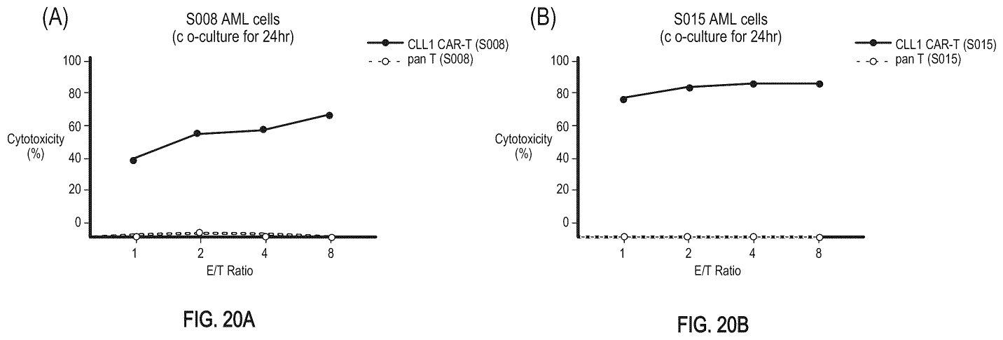

Isolated BMMCs from patient S-008 and S015 were incubated with their corresponding r/rAML patients' CLL-1 (61H08 HL_2828z) CAR-T cells at E/T ratios of 1, 2, 4 and 8 for 24 hrs. The absolute AML blasts (CD45dim CD34+CD38+) of each culture were counted by ratio comparison with counting beads by flow cytometry. It was shown that CLL-1 CAR-T cell candidate mediated cytotoxicity toward autologous primary AML blasts as compared to the corresponding untransduced T cells. R/R AML patient-derived CLL-1 CAR-T cell-mediated cytotoxicity is illustrated in FIG. 20A-B.

As shown in the aforementioned examples, a CAR-T cell against CLL-1 antigens was designed with the second-generation CAR format, and optimized by antibody variable region selection and domain (VH-VL) swapping, and by co-stimulatory domain (CD28 or 4-1BB) exchange. The CAR-T cell candidates, 61H08 HL_2828z and 61H08 LH_2828z, were selected with high CAR % (>80%), regular CD4/CD8 ratio, high cell expansion fold, high percentages of memory-like phenotypes of T_{scm} and T_{cm} (>70%), profound effector cytokine release, and the superior cytotoxicity against AML tumor cells both in vitro and in vivo. In AML U937-Luc xenograft animal model, CLL-1 (61H08 HL or LH) CAR-T cell with CD28 co-stimulatory domain exhibited better anti-tumor activity than those with 4-1BB domain, and prolonged the survival time and CAR-T cell persistence more than 43 days post CAR-T cell infusion. The cytokine release profiles of xenograft mice receiving CAR-T cells exhibited the profound effector cytokine release, including IFN-γ, granzyme A, perforin and granulysin. Furthermore, xenograft mice receiving 3×10⁵ CAR-T cells per mouse were able to elicit effective recall responses after tumor cell rechallenge. In the aspect of on-target off-tumor toxicity of CAR-T cells, hematotoxicity assessment revealed that CLL-1 CAR-T cell candidates, 61H08 HL 2828z and 61H08 LH_2828z, were tolerated as they exerted no or limited colony formation inhibition of CD34+ cells derived from bone marrow (BM) or peripheral blood (PB). Moreover, autologous CLL-1 (61H08 HL_2828z) CAR-T cells were successfully prepared from r/r AML patients and were demonstrated to mediate cytotoxicity to their paired AML blasts isolated from bone marrows. Thus, the CLL-1 CAR-T cell candidates, i.e., 61H08 HL 2828z and 61H08 HL 2828z have demonstrated the anti-AML potency with minimum safety concern, and will be further developed for the treatment of relapsed or refractory AML patients.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A chimeric antigen receptor (CAR) for Human C-type lectin-like molecule-1 (CLL-1) comprising a polypeptide comprising:
an extracellular antigen binding domain comprising a single heavy chain variable domain (VH) and a single light chain variable domain (VL);
a transmembrane domain; and
an intracellular signaling domain,
wherein the single heavy chain variable domain comprises a CDR1, a CDR2 and a CDR3 as set forth in a first amino acid sequence selected from the group consisting of SEQ ID NO: 10, SEQ ID NO: 12, SEQ ID NO: 14, SEQ ID NO: 16, and SEQ ID NO: 18, and
wherein the single light chain variable domain comprises a CDR1, a CDR2, and a CDR3 as set forth in a second amino acid sequence selected from the group consisting of SEQ ID NO: 11, SEQ ID NO: 13, SEQ ID NO: 15, SEQ ID NO: 17, and SEQ ID NO: 19.

2. The CAR of claim 1, wherein the first amino acid sequence is of SEQ ID NO: 14.

3. The CAR of claim 1, wherein the second amino acid sequence is of SEQ ID NO: 15.

4. The CAR of claim 1, wherein the single heavy chain variable domain is located at a N-terminus side of the single light chain variable domain.

5. The CAR of claim 1, wherein the single heavy chain variable domain is located at a C-terminus side of the single light chain variable domain.

6. The CAR of claim 1, wherein the single heavy chain variable domain and single light chain variable domain are directly fused to each other via a peptide bond.

7. The CAR of claim 1, wherein the single heavy chain variable domain and the single light chain variable domain are linked to each other via a peptide linker.

8. The CAR of claim 7, wherein the peptide linker comprises no more than 50 amino acid residues.

9. The CAR of claim 1, wherein the transmembrane domain is derived from CD8 or CD28.

10. The CAR of claim 1, wherein the intracellular signaling domain comprises a primary intracellular signaling domain of an immune effector cell.

11. The CAR of claim 10, wherein the primary intracellular signaling domain is derived from CD3ζ.

12. The CAR of claim 1, wherein the intracellular signaling domain comprises a co-stimulatory signaling domain.

13. The CAR of claim 12, wherein the co-stimulatory signaling domain is derived from a molecule selected from the group consisting of CD28, 4-1BB, OX40, ICOS and combinations thereof.

14. The CAR of claim 12, wherein the co-stimulatory signaling domain is derived from CD28, 4-1BB, or a combination thereof.

15. The CAR of claim 12, wherein the co-stimulatory signaling domain comprises a cytoplasmic domain of CD28.

16. The CAR of claim 1, further comprising a hinge domain.

17. The CAR of claim 16, wherein the hinge domain is located between a C-terminus of the extracellular antigen binding domain and a N-terminus of the transmembrane domain.

18. The CAR of claim 16, wherein the hinge domain is derived from molecule selected from the group consisting of CD28, 4-1BB, OX40, ICOS and combinations thereof.

19. The CAR of claim 16, wherein the hinge domain is derived from CD28.

20. The CAR of claim 1, further comprising a signal peptide located at a N-terminus of the polypeptide.

21. The CAR of claim 20, wherein the signal peptide is derived from CD28.

22. The CAR of claim 1, wherein the single heavy chain variable domain comprises a CDR1, a CDR2 and a CDR3 as set forth in an amino acid sequence of SEQ ID NO: 14, and wherein the single light chain variable domain comprises a CDR1, a CDR2, and a CDR3 as set forth in an amino acid sequence of SEQ ID NO: 15.

23. The CAR of claim 22, wherein the transmembrane domain is derived from CD8 or CD28.

24. The CAR of claim 23, wherein the intracellular signaling domain comprises a primary intracellular signaling domain of an immune effector cell, and wherein the primary intracellular signaling domain is derived from CD3ζ.

25. The CAR of claim 24, wherein the intracellular signaling domain comprises a co-stimulatory signaling domain, and wherein the co-stimulatory signaling domain is derived from CD28, 4-1BB, or a combination thereof.

26. The CAR of claim 1, wherein the polypeptide comprises an amino acid sequence selected from the group consisting of SEQ ID NOs: 20 to 26.

27. An immune effector cell comprising the CAR of claim 26.

28. A pharmaceutical composition comprising the immune effector cell of claim 27, and a pharmaceutically acceptable carrier

29. A method of treating a cancer that expresses CLL-1 in an individual, comprising administering to the individual an effective amount of the immune effector cell of claim 27.

30. The method of claim 29, wherein the cancer is AML.


SEQUENCE LISTING (74 sequences)

SEQ ID NO: 1 - protein, 22 aa, synthetic construct
MLLLVTSLLLCELPHPAFLLIP

SEQ ID NO: 2 - protein, 15 aa, synthetic construct
GGGGSGGGGSGGGGS

SEQ ID NO: 3 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 4 - protein, 66 aa, synthetic construct
IEVMYPPPYLDNEKSNGTIIHVKGKHLCPSPLFPGPSKPFWVLVVVGGVLACYSLLVTVAFIIFWV

SEQ ID NO: 5 - protein, 42 aa, synthetic construct
KRGRKKLLYIFKQPFMRPVQTTQEEDGCSCRFPEEEEGGCEL

SEQ ID NO: 6 - protein, 41 aa, synthetic construct
RSKRSRLLHSDYMNMTPRRPGPTRKHYQPYAPPRDFAAYRS

SEQ ID NO: 7 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 18 aa, synthetic construct
EGRGSLLTCGDVEENPGP

SEQ ID NO: 9 - protein, 233 aa, Homo sapiens
AAAPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 119 aa, synthetic construct
EVQLVESGGGLVQPGGSLRLSCAASGFTFTDYYIHWVRQAPGKGLEWVSMISPSGGGEAYYADSVKGRFTISRDNSKNTLYLQMNSLRAEDTAVYYCARAHDFDDFDYWGQGTLVTVSS

SEQ ID NO: 11 - protein, 108 aa, synthetic construct
DIQMTQSPSSLSASVGDRVTITCRASQSIRYSLAWYQQKPGKASKLLIYDASTLQSGVPSRFSGSGSGTDFTLTISSLQPEDFATYYCQQYNNSPLTFGQGTKVEIKR

SEQ ID NO: 12 - protein, 120 aa, synthetic construct
EVQLVESGGGLVQPGGSLRLSCAASGFTFSNHWMHWVRQAPGKGLEWVSSISGAGGGKSYADSVKGRFTISRDNSKNTLYLQMNSLRAEDTAVYYCARDESSYAYDGDYWGQGTLVTVSS

SEQ ID NO: 13 - protein, 108 aa, synthetic construct
DIQMTQSPSSLSASVGDRVTITCRANQSIRNYLNWYQQKPGKAPKLLIYAASTLQSGVPSRFSGSGSGTDFTLTISSLQPEDFATYYCQQNTSSPSTFGQGTKVEIKR

SEQ ID NO: 14 - protein, 120 aa, synthetic construct
EVQLVESGGGLVQPGGSLRLSCAASGFTFDDYGMHWVRQAPGKGLEWVSGIWPSGGNKEYADSVKGRFTISRDNSKNTLYLQMNSLRAEDTAVYYCARLLALTYDYFDYWGQGTLVTVSS

SEQ ID NO: 15 - protein, 108 aa, synthetic construct
DIQMTQSPSSLSASVGDRVTITCRASQGISSYLAWYQQKPGKAPKLLIYDASTLQSGVPSRFSGSGSGTDFTLTISSLQPEDFATYYCQQSYSTPFTFGQGTKVEIKR

SEQ ID NO: 16 - protein, 120 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAAYYCARSPLQWLDDAFDIWGQGTMVTVSS

SEQ ID NO: 17 - protein, 112 aa, synthetic construct
QSVLTQPPSVSAAPGQRVTISCSGSSSNIGSNYVSWYQQLPGTAPKLLIYDNNERPSGIPDRFSGSKSATSATLDITGLQTGDEADYFCGTWDNSPSTDWVFGGGTKVTVLG

SEQ ID NO: 18 - protein, 123 aa, synthetic construct
EVQLVESGGGLVQPGGSLRLSCAASGFTFSVNAMHWVRQAPGKGLEWVSTISGSDGHKYYADSVKSRFTISRDNSKNTLYLQMNSLRAEDTAVYYCARDVDSEIGDGYHEDIWGQGTLVTVSS

SEQ ID NO: 19 - protein, 108 aa, synthetic construct
DIQMTQSPSSLSASVGDRVTITCRASQSVTNALNWYQQKPGKAPKLLIYAASNLQSGVPSRFSGSGSGTDFTLTISSLQPEDFATYYCQQTNNSPATFGQGTKVEIKR

SEQ ID NO: 20 - protein, 506 aa, synthetic construct
MLLLVTSLLLCELPHPAFLLIPEVQLVESGGGLVQPGGSLRLSCAASGFTFDDYGMHWVRQAPGKGLEWVSGIWPSGGNKEYADSVKGRFTISRDNSKNTLYLQMNSLRAEDTAVYYCARLLALTYDYFDYWGQGTLVTVSSGGGGSGGGGSGGGGSDIQMTQSPSSLSASVGDRVTITCRASQGISSYLAWYQQKPGKAPKLLIYDASTLQSGVPSRFSGSGSGTDFTLTISSLQPEDFATYYCQQSYSTPFTFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGP

SEQ ID NO: 21 - protein, 506 aa, synthetic construct
MLLLVTSLLLCELPHPAFLLIPDIQMTQSPSSLSASVGDRVTITCRASQGISSYLAWYQQKPGKAPKLLIYDASTLQSGVPSRFSGSGSGTDFTLTISSLQPEDFATYYCQQSYSTPFTFGQGTKVEIKRGGGGSGGGGSGGGGSEVQLVESGGGLVQPGGSLRLSCAASGFTFDDYGMHWVRQAPGKGLEWVSGIWPSGGNKEYADSVKGRFTISRDNSKNTLYLQMNSLRAEDTAVYYCARLLALTYDYFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGP

SEQ ID NO: 22 - protein, 510 aa, synthetic construct
MLLLVTSLLLCELPHPAFLLIPQVQLQQWGAGLLKPSETLSLTCAVYGGSFSGYYWSWIRQPPGKGLEWIGEINHSGSTNYNPSLKSRVTISVDTSKNQFSLKLSSVTAADTAAYYCARSPLQWLDDAFDIWGQGTMVTVSSGGGGSGGGGSGGGGSQSVLTQPPSVSAAPGQRVTISCSGSSSNIGSNYVSWYQQLPGTAPKLLIYDNNERPSGIPDRFSGSKSATSATLDITGLQTGDEADYFCGTWDNSPSTDWVFGGGTKVTVLGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGP

SEQ ID NO: 23 - protein, 510 aa, synthetic construct
MLLLVTSLLLCELPHPAFLLIPQSVLTQPPSVSAAPGQRVTISCSGSSSNIGSNYVSWYQQLPGTAPKLLIYDNNERPSGIPDRFSGSKSATSATLDITGLQTGDEADYFCGTWDNSPSTDWVFGGGTKVTVLGGGGGSGGGGSGGGGSQVQLQQWGAGLLKPSETLSLTCAVYGGSFSGYYWSWIRQPPGKGLEWIGEINHSGSTNYNPSLKSRVTISVDTSKNQFSLKLSSVTAADTAAYYCARSPLQWLDDAFDIWGQGTMVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGP

SEQ ID NO: 24 - protein, 509 aa, synthetic construct
MLLLVTSLLLCELPHPAFLLIPEVQLVESGGGLVQPGGSLRLSCAASGFTFSVNAMHWVRQAPGKGLEWVSTISGSDGHKYYADSVKSRFTISRDNSKNTLYLQMNSLRAEDTAVYYCARDVDSEIGDGYHEDIWGQGTLVTVSSGGGGSGGGGSGGGGSDIQMTQSPSSLSASVGDRVTITCRASQSVTNALNWYQQKPGKAPKLLIYAASNLQSGVPSRFSGSGSGTDFTLTISSLQPEDFATYYCQQTNNSPATFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGP

SEQ ID NO: 25 - protein, 502 aa, synthetic construct
MLLLVTSLLLCELPHPAFLLIPEVQLVESGGGLVQPGGSLRLSCAASGFTFDDYGMHWVRQAPGKGLEWVSGIWPSGGNKEYADSVKGRFTISRDNSKNTLYLQMNSLRAEDTAVYYCARLLALTYDYFDYWGQGTLVTVSSGGGGSGGGGSGGGGSDIQMTQSPSSLSASVGDRVTITCRASQGISSYLAWYQQKPGKAPKLLIYDASTLQSGVPSRFSGSGSGTDFTLTISSLQPEDFATYYCQQSYSTPFTFGQGTKVEIKRIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGP

SEQ ID NO: 26 - protein, 502 aa, synthetic construct
MLLLVTSLLLCELPHPAFLLIPDIQMTQSPSSLSASVGDRVTITCRASQGISSYLAWYQQKPGKAPKLLIYDASTLQSGVPSRFSGSGSGTDFTLTISSLQPEDFATYYCQQSYSTPFTFGQGTKVEIKRGGGGSGGGGSGGGGSEVQLVESGGGLVQPGGSLRLSCAASGFTFDDYGMHWVRQAPGKGLEWVSGIWPSGGNKEYADSVKGRFTISRDNSKNTLYLQMNSLRAEDTAVYYCARLLALTYDYFDYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGP

SEQ ID NO: 27 - DNA, 1518 bp, synthetic construct
  source 1..1518: mol_type = other DNA
atgctgctgctggttacatctctgctgctgtgcgagctgccccatcctgcctttctgctgatccctgaagttcagctggttgaaagcggcggtggtctggttcagcctggtggcagcctgcgcctgagctgtgcagcaagcggctttacctttgatgattacggtatgcattgggttcgccaggcacctggcaaaggcctggaatgggttagcggtatttggccatctggtggtaacaaagaatatgcagatagcgttaaaggccgctttaccattagccgcgataatagcaaaaataccctgtacctgcaaatgaacagcctgcgcgcagaggataccgccgtgtactattgtgcccgcctgctggcactgacctacgattactttgattactggggccagggcaccctggtcaccgtgagctctggtggaggcggttcaggcggaggtggctctggcggtggcggatcggatatccagatgacacagagccctagcagcctgtctgccagcgtgggcgatcgcgtgaccatcacctgtagagccagccagggtatctcttcttacctggcatggtatcagcagaagcctggcaaagcccctaaactgctgatctatgatgcatctaccctgcagtctggcgtgccaagccgcttttctggcagcggctctggcaccgacttcaccctgaccatctctagcctgcagcctgaagacttcgccacctattattgccagcagtcttactctaccccatttacctttggccagggcaccaaagtggaaatcaaacgcaccacaacacccgctcctcggcctccaacaccagctccaacaattgccagccagcctctgtctctgaggcccgaagcttgtagaccagctgctggcggagccgtgcatacaagaggactggatttcgcctgcgacatctacatctgggctcctctggctggaacctgtggcgtgctgttgctgagcctggtcatcaccctgtactgcaagcggggcagaaagaagctgctgtacatctttaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccacaaaggatacctatgacgccctgcacatgcaggccctgcctccaagagaaggcagaggctctctgctgacatgcggcgacgtggaagagaaccctggacct

SEQ ID NO: 28 - DNA, 1518 bp, synthetic construct
  source 1..1518: mol_type = other DNA
atgctgctgctggttacatctctgctgctgtgcgagctgccccatcctgcctttctgctgatccctgatatccagatgacacagagccctagcagcctgtctgccagcgtgggcgatcgcgtgaccatcacctgtagagccagccagggtatctcttcttacctggcatggtatcagcagaagcctggcaaagcccctaaactgctgatctatgatgcatctaccctgcagtctggcgtgccaagccgcttttctggcagcggctctggcaccgacttcaccctgaccatctctagcctgcagcctgaagacttcgccacctattattgccagcagtcttactctaccccatttacctttggccagggcaccaaagtggaaatcaaacgcggtggaggcggttcaggcggaggtggctctggcggtggcggatcggaagttcagctggttgaaagcggcggtggtctggttcagcctggtggcagcctgcgcctgagctgtgcagcaagcggctttacctttgatgattacggtatgcattgggttcgccaggcacctggcaaaggcctggaatgggttagcggtatttggccatctggtggtaacaaagaatatgcagatagcgttaaaggccgctttaccattagccgcgataatagcaaaaataccctgtacctgcaaatgaacagcctgcgcgcagaggataccgccgtgtactattgtgcccgcctgctggcactgacctacgattactttgattactggggccagggcaccctggtcaccgtgagctctaccacaacacccgctcctcggcctccaacaccagctccaacaattgccagccagcctctgtctctgaggcccgaagcttgtagaccagctgctggcggagccgtgcatacaagaggactggatttcgcctgcgacatctacatctgggctcctctggctggaacctgtggcgtgctgttgctgagcctggtcatcaccctgtactgcaagcggggcagaaagaagctgctgtacatctttaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccacaaaggatacctatgacgccctgcacatgcaggccctgcctccaagagaaggcagaggctctctgctgacatgcggcgacgtggaagagaaccctggacct

SEQ ID NO: 29 - DNA, 1530 bp, synthetic construct
  source 1..1530: mol_type = other DNA
atgctgctgctggttacatctctgctgctgtgcgagctgccccatcctgcctttctgctgatccctcaggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgcgtattactgtgcgagatccccccttcagtggctggatgatgcttttgatatctggggccaagggacaatggtcaccgtctcttcaggtggaggcggttcaggcggaggtggctctggcggtggcggatcgcagtctgtgttgacgcagccgccctcagtgtctgcggccccaggacagagggtcaccatctcctgctctggaagcagctccaacattgggagtaattatgtatcctggtaccagcagctcccaggaacagcccccaaactcctcatttatgacaataatgagcgaccctcagggattcctgaccgattctctggctccaagtctgccacgtcagccaccctggacatcaccggactccagactggggacgaggccgattatttctgcggaacatgggataacagcccgagtactgattgggtgttcggcggagggaccaaggtgaccgtcctaggtaccacaacacccgctcctcggcctccaacaccagctccaacaattgccagccagcctctgtctctgaggcccgaagcttgtagaccagctgctggcggagccgtgcatacaagaggactggatttcgcctgcgacatctacatctgggctcctctggctggaacctgtggcgtgctgttgctgagcctggtcatcaccctgtactgcaagcggggcagaaagaagctgctgtacatctttaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccacaaaggatacctatgacgccctgcacatgcaggccctgcctccaagagaaggcagaggctctctgctgacatgcggcgacgtggaagagaaccctggacct

SEQ ID NO: 30 - DNA, 1530 bp, synthetic construct
  source 1..1530: mol_type = other DNA
atgctgctgctggttacatctctgctgctgtgcgagctgccccatcctgcctttctgctgatccctcagtctgtgttgacgcagccgccctcagtgtctgcggccccaggacagagggtcaccatctcctgctctggaagcagctccaacattgggagtaattatgtatcctggtaccagcagctcccaggaacagcccccaaactcctcatttatgacaataatgagcgaccctcagggattcctgaccgattctctggctccaagtctgccacgtcagccaccctggacatcaccggactccagactggggacgaggccgattatttctgcggaacatgggataacagcccgagtactgattgggtgttcggcggagggaccaaggtgaccgtcctaggtggtggaggcggttcaggcggaggtggctctggcggtggcggatcgcaggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgcgtattactgtgcgagatccccccttcagtggctggatgatgcttttgatatctggggccaagggacaatggtcaccgtctcttcaaccacaacacccgctcctcggcctccaacaccagctccaacaattgccagccagcctctgtctctgaggcccgaagcttgtagaccagctgctggcggagccgtgcatacaagaggactggatttcgcctgcgacatctacatctgggctcctctggctggaacctgtggcgtgctgttgctgagcctggtcatcaccctgtactgcaagcggggcagaaagaagctgctgtacatctttaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccacaaaggatacctatgacgccctgcacatgcaggccctgcctccaagagaaggcagaggctctctgctgacatgcggcgacgtggaagagaaccctggacct

SEQ ID NO: 31 - DNA, 1527 bp, synthetic construct
  source 1..1527: mol_type = other DNA
atgctgctgctggttacatctctgctgctgtgcgagctgccccatcctgcctttctgctgatccctgaagttcagctggttgaaagcggcggtggtctggttcagcctggtggcagcctgcgcctgagctgtgcagcaagcggctttaccttttctgttaacgcaatgcattgggttcgccaggcacctggcaaaggcctggaatgggttagcacaatttctggttctgatggtcataaatactatgcagatagcgttaaaagccgctttaccattagccgcgataatagcaaaaataccctgtacctgcaaatgaacagcctgcgcgcagaggataccgccgtgtactattgtgcccgcgatgttgattctgaaatcggtgatggttaccatgaagatatctggggccagggcaccctggtcaccgtgagctctggtggaggcggttcaggcggaggtggctctggcggcggcggatcggatatccagatgacacagagccctagcagcctgtctgccagcgtgggcgatcgcgtgaccatcacctgtagagccagccagtctgttaccaacgcactgaactggtatcagcagaagcctggcaaagcccctaaactgctgatctatgcagcatctaacctgcagtctggcgtgccaagccgcttttctggcagcggctctggcaccgacttcaccctgaccatctctagcctgcagcctgaagacttcgccacctattattgccagcagaccaacaactctccagcaacctttggccagggcaccaaagtggaaatcaaacgcaccacaacacccgctcctcggcctccaacaccagctccaacaattgccagccagcctctgtctctgaggcccgaagcttgtagaccagctgctggcggagccgtgcatacaagaggactggatttcgcctgcgacatctacatctgggctcctctggctggaacctgtggcgtgctgttgctgagcctggtcatcaccctgtactgcaagcggggcagaaagaagctgctgtacatctttaagcagcccttcatgcggcccgtgcagaccacacaagaggaagatggctgctcctgcagattccccgaggaagaagaaggcggctgcgagctgagagtgaagttcagcagatccgccgacgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccacaaaggatacctatgacgccctgcacatgcaggccctgcctccaagagaaggcagaggctctctgctgacatgcggcgacgtggaagagaaccctggacct

SEQ ID NO: 32 - DNA, 1506 bp, synthetic construct
  source 1..1506: mol_type = other DNA
atgctgctgctggttacatctctgctgctgtgcgagctgccccatcctgcctttctgctgatccctgaagttcagctggttgaaagcggcggtggtctggttcagcctggtggcagcctgcgcctgagctgtgcagcaagcggctttacctttgatgattacggtatgcattgggttcgccaggcacctggcaaaggcctggaatgggttagcggtatttggccatctggtggtaacaaagaatatgcagatagcgttaaaggccgctttaccattagccgcgataatagcaaaaataccctgtacctgcaaatgaacagcctgcgcgcagaggataccgccgtgtactattgtgcccgcctgctggcactgacctacgattactttgattactggggccagggcaccctggtcaccgtgagctctggtggaggcggttcaggcggaggtggctctggcggtggcggatcggatatccagatgacacagagccctagcagcctgtctgccagcgtgggcgatcgcgtgaccatcacctgtagagccagccagggtatctcttcttacctggcatggtatcagcagaagcctggcaaagcccctaaactgctgatctatgatgcatctaccctgcagtctggcgtgccaagccgcttttctggcagcggctctggcaccgacttcaccctgaccatctctagcctgcagcctgaagacttcgccacctattattgccagcagtcttactctaccccatttacctttggccagggcaccaaagtggaaatcaaacgcattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcagatccgccgacgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccacaaaggatacctatgacgccctgcacatgcaggccctgcctccaagagaaggcagaggctctctgctgacatgcggcgacgtggaagagaaccctggacct

SEQ ID NO: 33 - DNA, 1506 bp, synthetic construct
  source 1..1506: mol_type = other DNA
atgctgctgctggttacatctctgctgctgtgcgagctgccccatcctgcctttctgctgatccctgatatccagatgacacagagccctagcagcctgtctgccagcgtgggcgatcgcgtgaccatcacctgtagagccagccagggtatctcttcttacctggcatggtatcagcagaagcctggcaaagcccctaaactgctgatctatgatgcatctaccctgcagtctggcgtgccaagccgcttttctggcagcggctctggcaccgacttcaccctgaccatctctagcctgcagcctgaagacttcgccacctattattgccagcagtcttactctaccccatttacctttggccagggcaccaaagtggaaatcaaacgcggtggaggcggttcaggcggaggtggctctggcggtggcggatcggaagttcagctggttgaaagcggcggtggtctggttcagcctggtggcagcctgcgcctgagctgtgcagcaagcggctttacctttgatgattacggtatgcattgggttcgccaggcacctggcaaaggcctggaatgggttagcggtatttggccatctggtggtaacaaagaatatgcagatagcgttaaaggccgctttaccattagccgcgataatagcaaaaataccctgtacctgcaaatgaacagcctgcgcgcagaggataccgccgtgtactattgtgcccgcctgctggcactgacctacgattactttgattactggggccagggcaccctggtcaccgtgagctctattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcagatccgccgacgctcccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccacaaaggatacctatgacgccctgcacatgcaggccctgcctccaagagaaggcagaggctctctgctgacatgcggcgacgtggaagagaaccctggacct

SEQ ID NO: 34 - protein, 10 aa, synthetic construct
GFTFTDYYIH

SEQ ID NO: 35 - protein, 10 aa, synthetic construct
GFTFSNHWMH

SEQ ID NO: 36 - protein, 10 aa, synthetic construct
GFTFSDYWMH

SEQ ID NO: 37 - protein, 10 aa, synthetic construct
GFTFDDYGMH

SEQ ID NO: 38 - protein, 10 aa, synthetic construct
GGSFSGYYWS

SEQ ID NO: 39 - protein, 10 aa, synthetic construct
GFTFSSYEMN

SEQ ID NO: 40 - protein, 10 aa, synthetic construct
GFTFSVNAMH

SEQ ID NO: 41 - protein, 18 aa, synthetic construct
MISPSGGGEAYYADSVKG

SEQ ID NO: 42 - protein, 17 aa, synthetic construct
SISGAGGGKSYADSVKG

SEQ ID NO: 43 - protein, 17 aa, synthetic construct
VISPGGSNIYYADSVKG

SEQ ID NO: 44 - protein, 17 aa, synthetic construct
GIWPSGGNKEYADSVKG

SEQ ID NO: 45 - protein, 16 aa, synthetic construct
EINHSGSTNYNPSLKS

SEQ ID NO: 46 - protein, 17 aa, synthetic construct
YISSSGSTIYYADSVKG

SEQ ID NO: 47 - protein, 17 aa, synthetic construct
TISGSDGHKYYADSVKS

SEQ ID NO: 48 - protein, 9 aa, synthetic construct
AHDFDDFDY

SEQ ID NO: 49 - protein, 11 aa, synthetic construct
DESSYAYDGDY

SEQ ID NO: 50 - protein, 9 aa, synthetic construct
TYRIEDFDY

SEQ ID NO: 51 - protein, 11 aa, synthetic construct
LLALTYDYFDY

SEQ ID NO: 52 - protein, 12 aa, synthetic construct
SPLQWLDDAFDI

SEQ ID NO: 53 - protein, 7 aa, synthetic construct
DYGDHYY

SEQ ID NO: 54 - protein, 14 aa, synthetic construct
DVDSEIGDGYHEDI

SEQ ID NO: 55 - protein, 11 aa, synthetic construct
RASQSIRYSLA

SEQ ID NO: 56 - protein, 11 aa, synthetic construct
RANQSIRNYLN

SEQ ID NO: 57 - protein, 11 aa, synthetic construct
RASQDMSSNLN

SEQ ID NO: 58 - protein, 11 aa, synthetic construct
RASQGISSYLA

SEQ ID NO: 59 - protein, 13 aa, synthetic construct
SGSSSNIGSNYVS

SEQ ID NO: 60 - protein, 11 aa, synthetic construct
QGDSLRSYYAS

SEQ ID NO: 61 - protein, 11 aa, synthetic construct
RASQSVTNALN

SEQ ID NO: 62 - protein, 7 aa, synthetic construct
DASTLQS

SEQ ID NO: 63 - protein, 7 aa, synthetic construct
AASTLQS

SEQ ID NO: 64 - protein, 7 aa, synthetic construct
NASTLQS

SEQ ID NO: 65 - protein, 7 aa, synthetic construct
DNNERPS

SEQ ID NO: 66 - protein, 7 aa, synthetic construct
GKNNRPS

SEQ ID NO: 67 - protein, 7 aa, synthetic construct
AASNLQS

SEQ ID NO: 68 - protein, 9 aa, synthetic construct
QQYNNSPLT

SEQ ID NO: 69 - protein, 9 aa, synthetic construct
QQNTSSPST

SEQ ID NO: 70 - protein, 8 aa, synthetic construct
QQYSTTPT

SEQ ID NO: 71 - protein, 9 aa, synthetic construct
QQSYSTPFT

SEQ ID NO: 72 - protein, 12 aa, synthetic construct
GTWDNSPSTDWV

SEQ ID NO: 73 - protein, 10 aa, synthetic construct
NSRDSSGNRV

SEQ ID NO: 74 - protein, 9 aa, synthetic construct
QQTNNSPAT